SEQUENCE LISTING (62 sequences)

SEQ ID NO: 1 - DNA, 9189 bp, Brevibacterium ammoniagenes
atgtcgttgacccccttgcataccttgtctaatgacagcactgctcccgcggtgctgtttgcgggtcagggttctgcatggcaaaaggccatcgctgatgccgcagccagccctcaccagggcgcacaattgcgcgacatcctaaaagaagttcgcacgaccaccggcccagtagcacgcatcattgcgtcgtcgtgccctggcgtttatgaacgcttggaagaacttgctcagacccccgctgaccaagcaccggtggccaaggaatatgacgcgtacccggcttactccatccccggcatcgtcctgggacaaattggtgccattgagcacctgcgcgagctgggcatcgatgtcgattccgcgcagttagcaggccactcccagggttcattaggtgttgcagccgttaaggatgcacgccaggccctggctattgctgttttgatgggtactgcagcagcggtgacccagggcgcgaatgattcccgctcccacatgctgtccgtgcgtggcgtaccacgtgagatggtcgaagaatacctcgctggtgacgctgcgattgccgtggtcaacggccgcgtgcactttgcactgtcgggtaccccagaggatctggctaagaccgagtccaacctcacccaggctgccgagtcctacaacgacgcgctggaagaacgccgcatcggcggctccgaaattaacccagtcttcgacgtattggccgtggcacttcctttccaccacgcatcactgcaggatgcagcggatctgaccgtggactacgccacccagtgtggcctggacgctgagcttgcacgcgagctggcagattccatcctggttcagccacatagctgggttgagaccgtggccggtctcaactccacctacctgctctccttagaccgtggtctgtcttcgttgactacacctttgattgccggcaccggcaaggttgtggttccagctgctacgccagcggagcgcgataacctggctaccccaggcactgagctgcctaccgcggtgaactacgagaagttctcaccaaagctcatctccttgcccaacggcaagtcctacactcagactcgtttctccgagtggaccggcatgtcccccatcattttgggcggcatgacgccgaccacgatggatccgggcatcgttgccgcagcggccaacggtggctactggtcagagatggccggtggcggtcagtactccgatgaagcttttaccatcaacaaagacggcatgatggagctgctggagccaggtcgcaccgcagcatttaacaccatgttctttgaccgctacctgtggaacctacagttcggtgtcacccgcattgttcccaaggcacgcgctaatggtgctgcgtttaccggcgtgaccatctccgctggtatcccagagctggatgaagccaaggaattgctggaccagctcacctccgatggcttcccatacatctctttcaagccgggcaccaccaagcagattcaagactgcatcgctatcgcagcggataaccccacccaccgcgtcatcatccaaattgaagacggccacgctggtggccaccactcctgggtggatctggatgaaatgctgctggctacctacgcatctgcccgtgagcacgacaacctggccatcactgttggtggcggcatccactccccagaccgcgcatcggaatacctgaccggtacctggtccaccaagtacggtttgcccatcatgccggttgatggtgtcttcttgggcaccgtagccatggcgaccaaggaagcaacggctaatgatgacgttaagcagttgctagttgataccccaggtatttccccagagaccaatggcggttgggtaggccgactagatgccgacggcggcgtctcctcctcccagtcccacctgttggctgacttgcacgagattgataactcgtttgccaaggcctcgcgcatgatcacctcgatcccgatcgaggagtatgacgagcgtcgcgacgagatcattgctgctctggacaagacctccaagccatacttcggtgacctgtcggagatgacctacgaggattgggtcgctcgtttcgcagagcgcgcctacccttgggtggatccaacctggcacgatcgtttccacgatctgctccagcgcgtagaagcgcgtctcaatgacgctgaccacggcgacatcgagaccctattccccacactcgacgactccgagaacgcaccagaggcagtagccaagctgctggctgcctacccgaatgcaaagaccaccaaggtcaacacccgcgatgaggcatggttccctacccttatccgcaagcacgtcaagccaatgccgtggaccaccgctattgacggtgacctgaaggaatggtttgccaaggacaccctgtggcaggcccaggacccacgctacgacgcagacggcgtacgcatcattccaggaccggtttcggttgctggtatcaccaagaagaatgagcccgtcgcaaacctgctcggtcgcttcgaagacgccaccaccgcagcgcttaacgatgccggcgtggcaccagttgagctctactcccgcttggcttctgccaagaatgcagaagagttcctgcgcaatgcaccaaccatcatgtggcacggtcacctcattgccaacccggcgtatgagctgccagaagaagcttttgacatcgtcgatgacggcgaaggctttgctattcgcatcaactctgactcctactgggataacctcccagaagagcagcgtccgttctacgtcaagcacgttgatatccccgttgcgctgtcggaagccgtagcaaccggtgcctcccctgttgttgatgacgcgcgtttgccaaaggcagtcttcgacctgctcgcaggcgttgctggtgtcgggtctatctctgagaccggcgataagatcaccgaactgccgaaggtcatcgaaggctctgtctccgaagaaaacccttacggcctggtggaatactcctttaccttgccttctaccctgctgaccgcacacaccgcggtaaccggcgctgccttgggcaccgccaacgcaggcaccccagatgcgctggttggcccctgctggccagcaatttacaccgcgctgggcaccggtcgattgaccgaagaacacggtgagccagccggcaccgacttcccggtcattgaaggcctgctcaacgcagtccacctcgaccacgtcgtcgatgtgcgtgttcctcttcacgaactcgcaaagggtgaaaagggcgaaggccgtcgcattgacgtcacctcccgctgtgcatccatcgcggaatccaactccggtcgcattgtcaccgtggaacttgagttgtgggatgccgcaactcaagaagttgtggcgacgcagatgcagcgctttgccatccgtggccgcgctaccggcacctccgttccggtttctgcaccatcctggggcggcggcaagtctcaggacaagattgagaccaccccacgttccttcgtggatcgcgccattgtcaccgcgccatcggatatgaccccattcgcgctggtctccggtgactacaacccaattcacacctccaccaacgccgcgcgcttggtcaacctcgacgccccactggtgcacggcatgtggctatctgccaccgcgcagcacctagctggcaaccacggcaccgtggtgggttggacctattccatgtacggcatggtccagctcaacgatgaagtagaaatcaccgtcgaacgcgtaggccgcaagggcattcacgcagcattcgaggtcacctgccgcatcgacggcgaagtagtctcccgcggccaggcgctcatggcacagccacgcaccgcttatgtctacccaggccagggcatccaggccgagggcatgggccgtggtgaccgcgatgcttcggcagcagcgcgtgaggtatggcgtcgtgcagaccgccacacccgcaccgcaatgggcttttctattcgccagatcatcgatgacaaccccaccgagctcgtcgttcgcggcaccaagttcgtccaccccaatggcgtgctgcacttaacgcagttcactcaggttgccctcgcagtcgttgcttatgcacaaaccgagcgcctgcgcgaagcagatgctctgggcaccaactccatgtacgccggtcactcactgggtgagtacaccgcgctggcatcgttggcgaatatctttgacctcgaagcggttatcgacatcgtctactcccgtggctctgccatgggcaccttggtcgaacgtgatgaaaacggtaactccaactacggcatgggcgcgctgcgtccaaacatgattggtgttcccgcagaccaggttgaggcctacatcgcgcagaccgcggaagaaactggcgaattcctcgaaatcgtcaactacaacatcgctggtcagcagtactccatcgcgggtaccaaggctggtttggccgccctgaagaaaaaggccaactccgtcaaggaccgtgcttatgtcacggttccaggcatcgatgtacctttccactcccaggtactgcgcgacggcgttcctgctttcgcagaaaagctcgatgaactgttgccagaaaccttggacctggacgccctggtcggccgctacgtgccgaacctggtggcgctgccattcgagctgacccaggaatttgtcgataaggtcaagcctttggctccttccggcaagctggataacctcaaggtcgaagacaccgatgagcaagcccttgctcgcctgctcatgattgagctattgtcctggcagttcgcatcacctgtgcgctggattgaaacccagcagctgctctttgaagaagtagaccagatcatcgaagtcggtctcgcggcatccccaacgctgaccaacttggccaagcgctccatggatatcgccggcgtggacctcccggtcttcaacgtcgaacgcgaccaagaccaggtcatgctccaagacgttcaggaagcaccagctgcctccttcgacgtcgaggaaggagaggccacctcttcgaccgcagcgtctgaaaccccaggtgaatccgctgcggcggcctcggataatacccaggccatcccatcggctgagccacaaacggtggcagaggcaccagcaccatccgccgcaccagctggcggcaccgctgccgcagatgctcctgacctgccatttaccgcagcagaagccatcatggttctgttcgctttccagaacaagatccgccaggaccagatcaatgactcggatacggtcgaagagctcaccaacggtgtctcctcccgccgtaaccaactgttgatggatatgtccgcagaaatcggcgtgcccgccattgacggtgcagccgatgctgacgtggcaaccttgcgtgagcgcgtcaagactgccgctccgggctactcgccattcggcaccgtcttgtctgaggctattaccgctcgtctgcgccagctcactggtgcagcaggcgtcaagccggcctacatttcagagcgcgtgaccggaacttggggcttgcctatgtcctgggcagcccacgttgaggctgaaatcttgctcggctcccgtgaagaagactccgtgcgcggtggctccttgtccaccgttccttccgcggcgtcgtcgaaggccgatgtcgatgcgcttgtcgatgccgcggtccaggccgtagccgcagcacacggcacctcggtatccatgggtgctgcgagtggcgccggcggcggtggagtcgtcgactccgcagccttggatgcttacgcagatatcgtcaccggtgaaaacggtgtcctcgctactgctgctcgccaggttctggctcagctgggcttggtcgaggaagcccctgagacccctgagaccgataacaccttgttcgagaccgtcgaggccgagctgggttccggttgggaaaagaccgttaccccatcctttgacgccaagcgcgcagtgcttttcgatgaccgctgggcgtctgctcgcgaagatctcgcccgcgtggcactcggcgagatcgacttgccagtcaagcgtttccagggaaccggagagaccatcgccaagcaagcggaatggtgggcggagaacaccgctgcttccactggtgcgcacgcgaaggcaacctctgccgagaccctgcatgctattgctgccgcagcgcgcgaagaactcgacggcgaattcgctggcgatgtcgcgttggtcaccggtgcagccccaggctccattgctaccgctctcgtagaacgcctgctggaaggcggcgcgaccgtcatcatgactgcgtcacgtgtcagccagtcccgtaaggaatttgcacgcaagctctacgctgcacacgcgattcctggcgctgccctgtgggttgttcctgcgaacttgagctcctaccgcgatgttgatgctctcattgactggattggtaatgagcagcgtgaatctgtcggcaacgaagtcaagatcaccaagccagcgttgaccccaaccttggccttcccattcgcggcaccttccgtgtccggttctgtggccgatgccggcccacaggctgaaaaccagactcgcctgctgctgtggtctgttgagcgcaccatcgctggtctgtccaacctggcgcagcaaggcgtggatacccgctgccacattgtgctgcctggttctccgaaccgcggcatgttcggtggcgacggcgcttacggcgaagtcaaggcagccttggacgctattttggccaagtggtctgcagaagcaggctggccagaaggtgttaccttggcacaagccaagattggctgggtctctggtacctccctgatgggcggcaacgacgttctgattccggcagcggaagccgctggcatccacgtgtgggacccagaagagatttcttcccagctcatctccctagcttccgaagaatcccgcgcgaaggcagccgaggctccactagagctggatctgaccggtggtctgggctcgtccaagatctccatctccgagctggctgcccaggcccgcgaggacgccgaggcacaagctgcttccggtgataatgcagacgcagctgcggaagctcctgcagccacgattccagcactgcctaatacccgttcagtagagctgcctgcagcgctaccggaaggtgaagtgggcgacgtaaccacggatctggatgacatggtcgtcatcgcaggtgtcggcgaagtctcctcgtggggttcgggccgtacccgctttgaggcagaatatggcttgcagcgcgatggcgctgtggacctgaccgccgctggtgtcttggaattggcatggatgaccggactgatttcctggtccaatgacccacgtccagcctggtacgacgaagagggcaccgaagtcgatgaagcagatatctacgctcgcttccgcgacgaggttgtagctcgctccggtatccgtaccttgaccgataagtacaacatggttgaccagggctccattgacctgacttctgtgttcttggaccgcgatatcgtcttcaccgttcctaccgaacaagaagcactcgatattgaagaagccgacccatcgtttaccaagctgcgcgaagtcgacggcgagtgggaagtcacccgtttgaagggtgccaccgcccgcgtgccacgcaaggcaacgttgactcgtaccgttgctggtcaaatgccggatcacttcgatgctgccaagtggggcattccagaccacatgctggatgcactcgaccgcatggccgtgtggaacctggtgaccgcagtcgatgcctttacccaggcgggctttagcccggctgagttgctgcaggttattcacccagcgcaggttgctaccacccagggcaccggtatcggcggcatggaatccctgcacaaggtcttcgtgacccgtctgctcggtgaagaccgtccttccgacatcctgcaggaagcactgcctaacgttattgcagcgcacaccatgcagtctttggtgggcggctacggttcgatgattcaccctatcggtgcttgtgccaccgctgcggtgtccatcgaagaaggcgtggacaagattgccctgggcaaggccgacctggtcgttgccggtggtatcgatgacgtccaagttgagtctttgaccggcttcggcgacatgaacgccaccgctgagaccaagaagatgaccgatcagggcattgatgaccgcttcatctcccgtgcgaatgaccgccgtcgtggcggcttcctcgaggcagaaggcggcggtaccgtgcttctggttcgcggttccctggctcgtgagatgggtctgccggtctacgcggtcgttgcgcacgcggcgtcctacggcgacggtgcccacacctccattcctgctccaggtttgggtgctttgggcgctggccgtggccggaagaactcccgcctggccaagggcttggctggtttgggtctgactccaaatgacgtctcggtactgtccaagcacgacacctcgaccaacgccaatgacccgaatgagtcggaactgcactccatcttgtggcctgctattggccgcgatgtggaccagccactgtttgtgatttcgcagaagtcactgactggtcactccaaggctggtgccgcgctgttccagaccggcggtttgattgacgtcttccgcacgggacgcattccagctaacctgtcgctggattgtgtggatccattgattgagccaaaggccacgaacttggtctggctacgctccccactagatgtggaagcagccaaccgcccggtcaaggccgcggcgctcacctcgctcggcttcggtcacgtcggtgcattgattgtctacgcgcacccaggtgtcttcgaggctgccgttgcccagcaggtttcggccgaggctgctgccgaatggcgcgagaaggcaaatgcccgcctcgccgccggtgcagcacgcttcgaagccggcatgattggcaaggaaaccttgttcgaggtcatcgacggccgccgcctgcctgacgcagcgggcaccgttgagattgagaactacggcccagtcgccgccgacaaggccgcagaaattgcgctcttgcttgacgacgacatccgtcttaccgccgaaggcactttccctccggcgaagtag

SEQ ID NO: 2 - protein, 3062 aa, Brevibacterium ammoniagenes
MSLTPLHTLSNDSTAPAVLFAGQGSAWQKAIADAAASPHQGAQLRDILKEVRTTTGPVARIIASSCPGVYERLEELAQTPADQAPVAKEYDAYPAYSIPGIVLGQIGAIEHLRELGIDVDSAQLAGHSQGSLGVAAVKDARQALAIAVLMGTAAAVTQGANDSRSHMLSVRGVPREMVEEYLAGDAAIAVVNGRVHFALSGTPEDLAKTESNLTQAAESYNDALEERRIGGSEINPVFDVLAVALPFHHASLQDAADLTVDYATQCGLDAELARELADSILVQPHSWVETVAGLNSTYLLSLDRGLSSLTTPLIAGTGKVVVPAATPAERDNLATPGTELPTAVNYEKFSPKLISLPNGKSYTQTRFSEWTGMSPIILGGMTPTTMDPGIVAAAANGGYWSEMAGGGQYSDEAFTINKDGMMELLEPGRTAAFNTMFFDRYLWNLQFGVTRIVPKARANGAAFTGVTISAGIPELDEAKELLDQLTSDGFPYISFKPGTTKQIQDCIAIAADNPTHRVIIQIEDGHAGGHHSWVDLDEMLLATYASAREHDNLAITVGGGIHSPDRASEYLTGTWSTKYGLPIMPVDGVFLGTVAMATKEATANDDVKQLLVDTPGISPETNGGWVGRLDADGGVSSSQSHLLADLHEIDNSFAKASRMITSIPIEEYDERRDEIIAALDKTSKPYFGDLSEMTYEDWVARFAERAYPWVDPTWHDRFHDLLQRVEARLNDADHGDIETLFPTLDDSENAPEAVAKLLAAYPNAKTTKVNTRDEAWFPTLIRKHVKPMPWTTAIDGDLKEWFAKDTLWQAQDPRYDADGVRIIPGPVSVAGITKKNEPVANLLGRFEDATTAALNDAGVAPVELYSRLASAKNAEEFLRNAPTIMWHGHLIANPAYELPEEAFDIVDDGEGFAIRINSDSYWDNLPEEQRPFYVKHVDIPVALSEAVATGASPVVDDARLPKAVFDLLAGVAGVGSISETGDKITELPKVIEGSVSEENPYGLVEYSFTLPSTLLTAHTAVTGAALGTANAGTPDALVGPCWPAIYTALGTGRLTEEHGEPAGTDFPVIEGLLNAVHLDHVVDVRVPLHELAKGEKGEGRRIDVTSRCASIAESNSGRIVTVELELWDAATQEVVATQMQRFAIRGRATGTSVPVSAPSWGGGKSQDKIETTPRSFVDRAIVTAPSDMTPFALVSGDYNPIHTSTNAARLVNLDAPLVHGMWLSATAQHLAGNHGTVVGWTYSMYGMVQLNDEVEITVERVGRKGIHAAFEVTCRIDGEVVSRGQALMAQPRTAYVYPGQGIQAEGMGRGDRDASAAAREVWRRADRHTRTAMGFSIRQIIDDNPTELVVRGTKFVHPNGVLHLTQFTQVALAVVAYAQTERLREADALGTNSMYAGHSLGEYTALASLANIFDLEAVIDIVYSRGSAMGTLVERDENGNSNYGMGALRPNMIGVPADQVEAYIAQTAEETGEFLEIVNYNIAGQQYSIAGTKAGLAALKKKANSVKDRAYVTVPGIDVPFHSQVLRDGVPAFAEKLDELLPETLDLDALVGRYVPNLVALPFELTQEFVDKVKPLAPSGKLDNLKVEDTDEQALARLLMIELLSWQFASPVRWIETQQLLFEEVDQIIEVGLAASPTLTNLAKRSMDIAGVDLPVFNVERDQDQVMLQDVQEAPAASFDVEEGEATSSTAASETPGESAAAASDNTQAIPSAEPQTVAEAPAPSAAPAGGTAAADAPDLPFTAAEAIMVLFAFQNKIRQDQINDSDTVEELTNGVSSRRNQLLMDMSAEIGVPAIDGAADADVATLRERVKTAAPGYSPFGTVLSEAITARLRQLTGAAGVKPAYISERVTGTWGLPMSWAAHVEAEILLGSREEDSVRGGSLSTVPSAASSKADVDALVDAAVQAVAAAHGTSVSMGAASGAGGGGVVDSAALDAYADIVTGENGVLATAARQVLAQLGLVEEAPETPETDNTLFETVEAELGSGWEKTVTPSFDAKRAVLFDDRWASAREDLARVALGEIDLPVKRFQGTGETIAKQAEWWAENTAASTGAHAKATSAETLHAIAAAAREELDGEFAGDVALVTGAAPGSIATALVERLLEGGATVIMTASRVSQSRKEFARKLYAAHAIPGAALWVVPANLSSYRDVDALIDWIGNEQRESVGNEVKITKPALTPTLAFPFAAPSVSGSVADAGPQAENQTRLLLWSVERTIAGLSNLAQQGVDTRCHIVLPGSPNRGMFGGDGAYGEVKAALDAILAKWSAEAGWPEGVTLAQAKIGWVSGTSLMGGNDVLIPAAEAAGIHVWDPEEISSQLISLASEESRAKAAEAPLELDLTGGLGSSKISISELAAQAREDAEAQAASGDNADAAAEAPAATIPALPNTRSVELPAALPEGEVGDVTTDLDDMVVIAGVGEVSSWGSGRTRFEAEYGLQRDGAVDLTAAGVLELAWMTGLISWSNDPRPAWYDEEGTEVDEADIYARFRDEVVARSGIRTLTDKYNMVDQGSIDLTSVFLDRDIVFTVPTEQEALDIEEADPSFTKLREVDGEWEVTRLKGATARVPRKATLTRTVAGQMPDHFDAAKWGIPDHMLDALDRMAVWNLVTAVDAFTQAGFSPAELLQVIHPAQVATTQGTGIGGMESLHKVFVTRLLGEDRPSDILQEALPNVIAAHTMQSLVGGYGSMIHPIGACATAAVSIEEGVDKIALGKADLVVAGGIDDVQVESLTGFGDMNATAETKKMTDQGIDDRFISRANDRRRGGFLEAEGGGTVLLVRGSLAREMGLPVYAVVAHAASYGDGAHTSIPAPGLGALGAGRGRKNSRLAKGLAGLGLTPNDVSVLSKHDTSTNANDPNESELHSILWPAIGRDVDQPLFVISQKSLTGHSKAGAALFQTGGLIDVFRTGRIPANLSLDCVDPLIEPKATNLVWLRSPLDVEAANRPVKAAALTSLGFGHVGALIVYAHPGVFEAAVAQQVSAEAAAEWREKANARLAAGAARFEAGMIGKETLFEVIDGRRLPDAAGTVEIENYGPVAADKAAEIALLLDDDIRLTAEGTFPPAK

SEQ ID NO: 3 - DNA, 459 bp, Brevibacterium ammoniagenes
gtgctcgacaaccgtgaagcgatgaccgtgggtgtggacttggtccacatccccggctttgccgagcaattgtcgcgccctggttcgacttttgagcaagtgttttcgccgttggaacgtcgtcatgtcaaacgcgccgtgacgctgcagcggatgctacgaattcgagccttgcgggttcacggactgagcacctggctgggcggtgggcggcaaaagaagcgttcatcaaggcgtggtcgcaagcgatctacgcaagccaccagtgattgaaccagacctggtgaacttcgcagagatcgaagtcttgcccgaccgctggggcagggtagcgctgcagcttaaaggtgaagttgctgcaaaacttcaggaatcaatagggacgtggagctggcgctgagcatcagccatgatggcgattacgccaccgcgcagtgcctgctgcggtaccagcggtaa

SEQ ID NO: 4 - protein, 152 aa, Brevibacterium ammoniagenes
MLDNREAMTVGVDLVHIPGFAEQLSRPGSTFEQVFSPLERRHAQTRRDAAADATNSSLAGSRTEHLAGRWAAKEAFIKAWSQAIYGKPPVIEPDLVNFAEIEVLPDRWGRVALQLKGEVAAKLQESIDVELALSISHDGDYATAQCLLRYQR

SEQ ID NO: 5 - DNA, 20 bp, Artificial Sequence
  Synthetic primer
ccagctcaacgatgaagtag

SEQ ID NO: 6 - DNA, 20 bp, Artificial Sequence
  Synthetic primer
tcgatgatctggtctacttc

SEQ ID NO: 7 - DNA, 27 bp, Artificial Sequence
  Synthetic primer
gtcgacatgctcgacaaccgtgaagcg

SEQ ID NO: 8 - DNA, 31 bp, Artificial Sequence
  Synthetic primer
agatcttcactggtggcttgccgtagatcgc

SEQ ID NO: 9 - DNA, 37 bp, Artificial Sequence
  Synthetic primer
tctagatgcatagttaacatgtcgttgacccccttgc

SEQ ID NO: 10 - DNA, 20 bp, Artificial Sequence
  Synthetic primer
ggtacgcgtcatattccttg

SEQ ID NO: 11 - DNA, 40 bp, Artificial Sequence
  Synthetic primer
caaggaatatgacgcgtaccctcgaggcagaaggcggcgg

SEQ ID NO: 12 - DNA, 37 bp, Artificial Sequence
  Synthetic primer
atgcatgttaacatgtctactttgtcctacttcgccg

SEQ ID NO: 13 - DNA, 33 bp, Artificial Sequence
  Synthetic primer
gtcgacatgcatatgctcgacaaccgtgaagcg

SEQ ID NO: 14 - DNA, 30 bp, Artificial Sequence
  Synthetic primer
agatctatgcattaccgctggtaccgcagc

SEQ ID NO: 15 - protein, 2073 aa, Schizosaccharomyces pombe
MVEAEQVHQSLRSLVLSYAHFSPSILIPASQYLLAAQLRDEFLSLHPAPSAESVEKEGAELEFEHELHLLAGFLGLIAAKEEETPGQYTQLLRIITLEFERTFLAGNEVHAVVHSLGLNIPAQKDVVRFYYHSCALIGQTTKFHGSALLDESSVKLAAIFGGQGYEDYFDELIELYEVYAPFAAELIQVLSKHLFTLSQNEQASKVYSKGLNVLDWLAGERPERDYLVSAPVSLPLVGLTQLVHFSVTAQILGLNPGELASRFSAASGHSQGIVVAAAVSASTDSASFMENAKVALTTLFWIGVRSQQTFPTTTLPPSVVADSLASSEGNPTPMLAVRDLPIETLNKHIETTNTHLPEDRKVSLSLVNGPRSFVVSGPARSLYGLNLSLRKEKADGQNQSRIPHSKRKLRFINRFLSISVPFHSPYLAPVRSLLEKDLQGLQFSALKVPVYSTDDAGDLRFEQPSKLLLALAVMITEKVVHWEEACGFPDVTHIIDFGPGGISGVGSLTRANKDGQGVRVIVADSFESLDMGAKFEIFDRDAKSIEFAPNWVKLYSPKLVKNKLGRVYVDTRLSRMLGLPPLWVAGMTPTSVPWQFCSAIAKAGFTYELAGGGYFDPKMMREAIHKLSLNIPPGAGICVNVIYINPRTYAWQIPLIRDMVAEGYPIRGVTIAAGIPSLEVANELISTLGVQYLCLKPGSVEAVNAVISIAKANPTFPIVLQWTGGRAGGHHSFEDFHSPILLTYSAIRRCDNIVLIAGSGFGGADDTEPYLTGEWSAAFKLPPMPFDGILFGSRLMVAKEAHTSLAAKEAIVAAKGVDDSEWEKTYDGPTGGIVTVLSELGEPIHKLATRGIMFWKELDDTIFSLPRPKRLPALLAKKQYIIKRLNDDFQKVYFPAHIVEQVSPEKFKFEAVDSVEDMTYAELLYRAIDLMYVTKEKRWIDVTLRTFTGKLMRRIEERFTQDVGKTTLIENFEDLNDPYPVAARFLDAYPEASTQDLNTQDAQFFYSLCSNPFQKPVPFIPAIDDTFEFYFKKDSLWQSEDLAAVVGEDVGRVAILQGPMAAKHSTKVNEPAKELLDGINETHIQHFIKKFYAGDEKKIPIVEYFGGVPPVNVSHKSLESVSVTEEAGSKVYKLPEIGSNSALPSKKLWFELLAGPEYTWFRAIFTTQRVAKGWKLEHNPVRRIFAPRYGQRAVVKGKDNDTVVELYETQSGNYVLAARLSYDGETIVVSMFENRNALKKEVHLDFLFKYEPSAGYSPVSEILDGRNDRIKHFYWALWFGEEPYPENASITDTFTGPEVTVTGNMIEDFCRTVGNHNEAYTKRAIRKRMAPMDFAIVVGWQAITKAIFPKAIDGDLLRLVHLSNSFRMVGSHSLMEGDKVTTSASIIAILNNDSGKTVTVKGTVYRDGKEVIEVISRFLYRGTFTDFENTFEHTQETPMQLTLATPKDVAVLQSKSWFQLLDPSQDLSGSILTFRLNSYVRFKDQKVKSSVETKGIVLSELPSKAIIQVASVDFQSVDCHGNPVIEFLKRNGKPIEQPVEFENGGYSVIQVMDEGYSPVFVTPPTNSPYAEVSGDYNPIHVSPTFAAFVELPGTHGITHGMYTSAAARRFVETYAAQNVPERVKHYEVTFVNMVLPNTELITKLSHTGMINGRKIIKVEVLNQETSEPVLVGTAEVEQPVSAYVFTGQGSQEQGMGMDLYASSPVARKIWDSADKHFLTNYGFSIIDIVKHNPHSITIHFGGSKGKKIRDNYMAMAYEKLMEDGTSKVVPVFETITKDSTSFSFTHPSGLLSATQFTQPALTLMEKSAFEDMRSKGLVQNDCAFAGHSLGEYSALSAMGDVLSIEALVDLVFLRGLTMQNAVHRDELGRSDYGMVAANPSRVSASFTDAALRFIVDHIGQQTNLLLEIVNYNVENQQYVVSGNLLSLSTLGHVLNFLKVQKIDFEKLKETLTIEQLKEQLTDIVEACHAKTLEQQKKTGRIELERGYATIPLKIDVPFHSSFLRGGVRMFREYLVKKIFPHQINVAKLRGKYIPNLTAKPFEISKEYFQNVYDLTGSQRIKKILQNWDEYESS

SEQ ID NO: 16 - DNA, 6232 bp, Schizosaccharomyces pombe
aagcttactatactgtgtagtagagagtgataaaatgttaattatgccacaagcagttgctaattcgctatatttgataacgatgcgttaaatattcgttacatgcttcaaagctgataggtttaacctgagtgttcccacgcgatagtaaaggatcaagttaactagaaccaacaactaaagcaggtgttggagttttgttaaaccatttgaataatgagaccagaagttgagcaggagcttgctcatactttattattggagttgcttgcataccagtttgcatctcctgtccgttggattgagacgcaagatgtaattctttctcctccagtatcggctgaacgtatcgtcgaaattggacctagtcctaccttagctggtatggctaagcgtaccttgaaattgaaatatgagaacatggatgccgctttaagtattaatcgtgaagttctttgctactctaaagatgctcgtgaaatctattacaactttgaggacgaggttgctgatgaacctgccgaagccccagcttcaaccagctccactccaaaggttgaaactgctgctgctgccgctcccgctgccacgccagcccctgccccagcacaaacatcagccccagctgctgctttacctgacgagcctcccaaagctcttgaggtacttcatactcttgttgcccaaaagttgaagaaaagcatcgaggaagtctcccctcaaaaatctatcaaagatttggttggcggtaagtccactttgcaaaacgaaattcttggtgatttacagaaggagttcggtgccactcccgagaagccagaggaggttccattggatgagcttggagctatcatgcagtcaagctttaacggatctcttggtaaacaatcgtcttctcttatctcacgaatgatttcctcaaaaatgcctggtggtttcaataattctgctgttcgtggttatttaggaaaccgttatggtttgggtcctggtcgtttggagtctgtgcttttgttagcgcttaccatggaacctgcatcacgtttgggctcggaagctgatgctaaagcttggcttgatagtgtagctcaaaaatatgctgctcgtaatggtgttacattatcttctcctactgctgaaggcggttcttcgtccggttctgcagctgttatcgatgaagaaacctttaagaaactcaccaagaataataccatgcttgttactcagcaattagaactatttgctcgatacctcaataaagaccttcgtgctggccaaaaggctcaagttgctgaaaaggttatttccgataccttacgcgctcaattagatttatggaacgaagaacatggtgaattttatgcatcaggaattgctcctattttttcgcctttaaaagctcgcgtttacgactccgactggaattgggctcgtcaagatgctcttaagatgttttttgacattatctttggtcgtcttaggcatgttgatactgaaatagtcgctcgttgtatttctgttatgaatagatccaaccctactttacttgaatttatgcaatatcatattgatcattgtcccgccgaaaagggtgaaacatatcaacttgctaaaaccttgggccaacagctaattgataattgcaaatccgtgatagatgctcctccagttttcaaaaatgtgaatcatccaactgctccttctacgacgattgacgaacgtggtaatttgaattatgaagaaatccctagaccaggtgttcgcaaattaactcattacgttactgagatggccaaaggtggtaaattaccaacggagtccaaaaacaaagctaaggtacaaaacgatttggctcgaatttatcgcattattaagtctcaaaacaaaatgtctcgttcgtctaagttgcagattaaacagttgtacggtcaggttttacatgccctttcccttccattgccttcttccaacgatgaacaaacgcctgttaaagaaaccattcctttccttcatattaggaagaagtccgttgatggtaattgggaattcaacaagtcattgactggcacttatttagatgttttagaatcgggtgctaagaatggtataacataccaagacaaatatgctctagtgactggtgcaggtgcaggctccattggtgctcagattgttgaaggtctccttgctggtggtgctaaagttgtagttactacatcccggttttcgcgcaaggttactgaattttatcaatccctttacacccgccatggaagccgtggttcatgtctgatcgtggttccatttaaccaaggatctaagacagacgtagaagctcttattgattatatttatgacgaaaagaagggtcttggatggaacttggactacattgttcctttcgctgccattccagaaaatggtcgtgaaattgatggcattgattctcgttccgagtttgctcaccgtattatgttgacaaacattttgagactgcttggcgccgtcaaaagtcaaaaggcctctcgtggtatggatacccgacccgctcaagttattttgcctctttctcccaatcacggtacctttggaaacgatggtttatactcggaatctaagttaggtttagaaactttgtttaaccgttggtactccgagtcatgggctaattacctaaccatttgtggggctgtcattggttggactcgtggtacaggcttaatggcacctaataatattgtttctcagggaatcgaaaaatatggtgttcgtactttttcgcagagtgagatggcttttaacattttgggtttgatgtcccagaaagtcgtcgacttgtgtcaatctgaaccaatttatgccaaccttaacggtggtcttgagcttttacctgatctcaaggacctttccactcgtttgcgtaccgaattgttagaaactgccgaaatccgccgcgctgttgccgcagagactgcctttgatcatagcattaccaacggacctgactctgaagcagttttccagaaaactgccattcagcctagggccaatcttaaatttaatttccccaaattgaaaccttatgaagccctttctcatttatctgatcttcgtggaatggttgatttagaaaaagttcctgttgttactggtttttccgaagtaggtccatggggtaactctcgtactagatgggatatggagtgttatggtgagttttcactagaaggatgtgtcgaaattgcttggattatgggattaattaaaaacttcaatggcaagggcaaagacggcaagccctattcaggttgggttgatacaaagaccggtgaacctgtggacgacaaagacgttaaagctaagtatgagaagtatatactggagcattgcggtatccgtattattgaagctgaactcttccatggatataatcctgaaaagaaagagcttttgcaagaagttgttattgatcatgacttagagccttttgaagcatccaaagaggctgctcatgagttcaagcttcgtcatggtgatcaagttgaaatttttgaaattcctgattctaccgaatggtccgtacgcttcaagcgcggtacaagtatgctaattcctaaggctttgcgctttgatcgatttgttgctggccagattccacttggttgggatcccaaacgttatggcattcctgacgatattatttctcaagttgaccctacaactttgtacgttttagtgtctactgtagaagctctggttgcatcaggtattacagatccttatgaatgctataagtatattcacgtatctgaacttggtaatacagttggttctggtattggtggtatgtctgctcttcgtggaatgtacaaggaccgctggactgataaacctgttcaaaaagatattttacaagaatcattcattaacactgccaatgcttggattaacatgcttttgctctctgcctctggtcctattaagactcctgttggtgcttgcgctaccgctgtcgaatctgttgatgcagctgtcgacttgatcacttctggtaaggccaggatatgtattagcggtggttatgacgacttttcagaagaaggttcatacgagtttgcgaacatgggtgctacatcaaatgctgctaaggaaacagaaaggggacgtactcctcaagaaatgtctcgtcctgctacttctactcgtgatggatttatggagtctcaaggtgctggtgtacagattatcatgcaagcaaagcttgctattgagatgggtgtccctatacatggtattgttggttatgtttccacagctatggataaacaaggtcgttcggttcctgcccctgggcaaggtattttgactggtgctcgtgaaatcgcgactaagacaccccttcccatagttgaccttaaattccgttctcgtcaactccaacgccgccgttctcaaattggtgaatgggccgaacgcgagtatctttatttagaagaagaacttgatgcgatgaaggttcaaaatcctgacttggatttagaggcttaccgtatagagcgtatcaacgttattaaggaggaggttgttcgacaagaaaaggaggcgctcaatacttttggaaatgaattttggaaacgtgatcctactattgctcctatccgtggtgcattagctgtttggggtcttactattgacgatttgggcgttgcatcattccatggtacctctaccaaagccaatgagaagaatgaatgcgatgtcattgacagtcagttaacacatctcggacgctctaagggtaacgctgtgtacggtgttttccagaaatatctcactggacatagcaagggtggtgctggagcttggatgctcaacggagctctccaaattcttcgctctgggtttgttccgggtaatcgtaacgccgataacattgatgagtatctagcacgattcgaccgggttatgttccctagtgaaggtatacaaactgatggcataaaggcagcatctgttactgcatttggttttggacaagttggtggacaagttatagttatccatcctgattacatttacggtgtgattgatgaggctacttataatgcttacaaagctaaaactgctgctcgttataaggcatcttatcgttacacccacgatgcgctggtttacaacaatttggtccgcgccaaggattctcctccttacaccaaagaacaagagaaagccgtttatctcaatcctttggcacgcgcttcgaagagcaaagctggcacttggactttccctgccacactgcctgctgaatccgacatttctaaaaccaacgaaactacacgtactctacaaagcctaacaacctcattgaccaactccaatgaaaatgttggcgtggatgttgaacttgtatcagcgattagcattgataatgagacctttatagaaaggaattttactgataccgagcgaaagtactgttttgcagctcctaatccccaagctagctttgccggacgttggtcagccaaagaggctgtctttaagtctttgggtatttccggtaaaggcgctgcagctccattgaaggatatcgaaattatttcttcagagtctggtgctcctgaagtagttttgcacggagaggctgcgaaggctgcaacgaccgccggtgtgaagagtgtttccgtcagtatttcccacgatgataatcaaagtgtcagtgttgctttggctcacaagtaatttacgttatattgtctttcaacattggtatgcggattttcgcattcccttcaatcgtttgatttaatacactatctttaatctttttgtttacctcaaatgctttgaaatggttatcgatttttgtagtcgttatatacgcagttagaaataaattacttttaaccttataaattattatgctctaaaaaaatgcagtatcattaaatttaaacgaatgtccttacacgtatgagtatttaaactgatattgagttatcttcataaatttctgaagccaggcagcggttgttgtttcatcgaaagaaatggggttcatatatgctgttgaagtgttttgctcgaacaaaatttcagttagatgcttaatcactgtccaaccgtaagcattcggaaaccgcctacgacaatcatggtattgtgtgcatcaccatcgagtctaacagcaacctttctccacgcaagcctatgccagtttttggcaatg

SEQ ID NO: 17 - protein, 1842 aa, Schizosaccharomyces pombe
MRPEVEQELAHTLLLELLAYQFASPVRWIETQDVILSPPVSAERIVEIGPSPTLAGMAKRTLKLKYENMDAALSINREVLCYSKDAREIYYNFEDEVADEPAEAPASTSSTPKVETAAAAAPAATPAPAPAQTSAPAAALPDEPPKALEVLHTLVAQKLKKSIEEVSPQKSIKDLVGGKSTLQNEILGDLQKEFGATPEKPEEVPLDELGAIMQSSFNGSLGKQSSSLISRMISSKMPGGFNNSAVRGYLGNRYGLGPGRLESVLLLALTMEPASRLGSEADAKAWLDSVAQKYAARNGVTLSSPTAEGGSSSGSAAVIDEETFKKLTKNNTMLVTQQLELFARYLNKDLRAGQKAQVAEKVISDTLRAQLDLWNEEHGEFYASGIAPIFSPLKARVYDSDWNWARQDALKMFFDIIFGRLRHVDTEIVARCISVMNRSNPTLLEFMQYHIDHCPAEKGETYQLAKTLGQQLIDNCKSVIDAPPVFKNVNHPTAPSTTIDERGNLNYEEIPRPGVRKLTHYVTEMAKGGKLPTESKNKAKVQNDLARIYRIIKSQNKMSRSSKLQIKQLYGQVLHALSLPLPSSNDEQTPVKETIPFLHIRKKSVDGNWEFNKSLTGTYLDVLESGAKNGITYQDKYALVTGAGAGSIGAQIVEGLLAGGAKVVVTTSRFSRKVTEFYQSLYTRHGSRGSCLIVVPFNQGSKTDVEALIDYIYDEKKGLGWNLDYIVPFAAIPENGREIDGIDSRSEFAHRIMLTNILRLLGAVKSQKASRGMDTRPAQVILPLSPNHGTFGNDGLYSESKLGLETLFNRWYSESWANYLTICGAVIGWTRGTGLMAPNNIVSQGIEKYGVRTFSQSEMAFNILGLMSQKVVDLCQSEPIYANLNGGLELLPDLKDLSTRLRTELLETAEIRRAVAAETAFDHSITNGPDSEAVFQKTAIQPRANLKFNFPKLKPYEALSHLSDLRGMVDLEKVPVVTGFSEVGPWGNSRTRWDMECYGEFSLEGCVEIAWIMGLIKNFNGKGKDGKPYSGWVDTKTGEPVDDKDVKAKYEKYILEHCGIRIIEAELFHGYNPEKKELLQEVVIDHDLEPFEASKEAAHEFKLRHGDQVEIFEIPDSTEWSVRFKRGTSMLIPKALRFDRFVAGQIPLGWDPKRYGIPDDIISQVDPTTLYVLVSTVEALVASGITDPYECYKYIHVSELGNTVGSGIGGMSALRGMYKDRWTDKPVQKDILQESFINTANAWINMLLLSASGPIKTPVGACATAVESVDAAVDLITSGKARICISGGYDDFSEEGSYEFANMGATSNAAKETERGRTPQEMSRPATSTRDGFMESQGAGVQIIMQAKLAIEMGVPIHGIVGYVSTAMDKQGRSVPAPGQGILTGAREIATKTPLPIVDLKFRSRQLQRRRSQIGEWAEREYLYLEEELDAMKVQNPDLDLEAYRIERINVIKEEVVRQEKEALNTFGNEFWKRDPTIAPIRGALAVWGLTIDDLGVASFHGTSTKANEKNECDVIDSQLTHLGRSKGNAVYGVFQKYLTGHSKGGAGAWMLNGALQILRSGFVPGNRNADNIDEYLARFDRVMFPSEGIQTDGIKAASVTAFGFGQVGGQVIVIHPDYIYGVIDEATYNAYKAKTAARYKASYRYTHDALVYNNLVRAKDSPPYTKEQEKAVYLNPLARASKSKAGTWTFPATLPAESDISKTNETTRTLQSLTTSLTNSNENVGVDVELVSAISIDNETFIERNFTDTERKYCFAAPNPQASFAGRWSAKEAVFKSLGISGKGAAAPLKDIEIISSESGAPEVVLHGEAAKAATTAGVKSVSVSISHDDNQSVSVALAHK

SEQ ID NO: 18 - protein, 2051 aa, Schizosaccharomyces pombe
MDAYSTRPLTLSHGSLEHVLLVPTASFFIASQLQEQFNKILPEPTEGFAADDEPTTPAELVGKFLGYVSSLVEPSKVGQFDQVLNLCLTEFENCYLEGNDIHALAAKLLQENDTTLVKTKELIKNYITARIMAKRPFDKKSNSALFRAVGEGNAQLVAIFGGQGNTDDYFEELRDLYQTYHVLVGDLIKFSAETLSELIRTTLDAEKVFTQGLNILEWLENPSNTPDKDYLLSIPISCPLIGVIQLAHYVVTAKLLGFTPGELRSYLKGATGHSQGLVTAVAIAETDSWESFFVSVRKAITVLFFIGVRCYEAYPNTSLPPSILEDSLENNEGVPSPMLSISNLTQEQVQDYVNKTNSHLPAGKQVEISLVNGAKNLVVSGPPQSLYGLNLTLRKAKAPSGLDQSRIPFSERKLKFSNRFLPVASPFHSHLLVPASDLINKDLVKNNVSFNAKDIQIPVYDTFDGSDLRVLSGSISERIVDCIIRLPVKWETTTQFKATHILDFGPGGASGLGVLTHRNKDGTGVRVIVAGTLDINPDDDYGFKQEIFDVTSNGLKKNPNWLEEYHPKLIKNKSGKIFVETKFSKLIGRPPLLVPGMTPCTVSPDFVAATTNAGYTIELAGGGYFSAAGMTAAIDSVVSQIEKGSTFGINLIYVNPFMLQWGIPLIKELRSKGYPIQFLTIGAGVPSLEVASEYIETLGLKYLGLKPGSIDAISQVINIAKAHPNFPIALQWTGGRGGGHHSFEDAHTPMLQMYSKIRRHPNIMLIFGSGFGSADDTYPYLTGEWSTKFDYPPMPFDGFLFGSRVMIAKEVKTSPDAKKCIAACTGVPDDKWEQTYKKPTGGIVTVRSEMGEPIHKIATRGVMLWKEFDETIFNLPKNKLVPTLEAKRDYIISRLNADFQKPWFATVNGQARDLATMTYEEVAKRLVELMFIRSTNSWFDVTWRTFTGDFLRRVEERFTKSKTLSLIQSYSLLDKPDEAIEKVFNAYPAAREQFLNAQDIDHFLSMCQNPMQKPVPFVPVLDRRFEIFFKKDSLWQSEHLEAVVDQDVQRTCILHGPVAAQFTKVIDEPIKSIMDGIHDGHIKKLLHQYYGDDESKIPAVEYFGGESPVDVQSQVDSSSVSEDSAVFKATSSTDEESWFKALAGSEINWRHASFLCSFITQDKMFVSNPIRKVFKPSQGMVVEISNGNTSSKTVVTLSEPVQGELKPTVILKLLKENIIQMEMIENRTMDGKPVSLPLLYNFNPDNGFAPISEVMEDRNQRIKEMYWKLWIDEPFNLDFDPRDVIKGKDFEITAKEVYDFTHAVGNNCEDFVSRPDRTMLAPMDFAIVVGWRAIIKAIFPNTVDGDLLKLVHLSNGYKMIPGAKPLQVGDVVSTTAVIESVVNQPTGKIVDVVGTLSRNGKPVMEVTSSFFYRGNYTDFENTFQKTVEPVYQMHIKTSKDIAVLRSKEWFQLDDEDFDLLNKTLTFETETEVTFKNANIFSSVKCFGPIKVELPTKETVEIGIVDYEAGASHGNPVVDFLKRNGSTLEQKVNLENPIPIAVLDSYTPSTNEPYARVSGDLNPIHVSRHFASYANLPGTITHGMFSSASVRALIENWAADSVSSRVRGYTCQFVDMVLPNTALKTSIQHVGMINGRKLIKFETRNEDDVVVLTGEAEIEQPVTTFVFTGQGSQEQGMGMDLYKTSKAAQDVWNRADNHFKDTYGFSILDIVINNPVNLTIHFGGEKGKRIRENYSAMIFETIVDGKLKTEKIFKEINEHSTSYTFRSEKGLLSATQFTQPALTLMEKAAFEDLKSKGLIPADATFAGHSLGEYAALASLADVMSIESLVEVVFYRGMTMQVAVPRDELGRSNYGMIAINPGRVAASFSQEALQYVVERVGKRTGWLVEIVNYNVENQQYVAAGDLRALDTVTNVLNFIKLQKIDIIELQKSLSLEEVEGHLFEIIDEASKKSAVKPRPLKLERGFACIPLVGISVPFHSTYLMNGVKPFKSFLKKNIIKENVKVARLAGKYIPNLTAKPFQVTKEYFQDVYDLTGSEPIKEIIDNWEKYEQS

SEQ ID NO: 19 - protein, 1887 aa, Saccharomyces cerevisiae
MKPEVEQELAHILLTELLAYQFASPVRWIETQDVFLKDFNTERVVEIGPSPTLAGMAQRTLKNKYESYDAALSLHREILCYSKDAKEIYYTPDPSELAAKEEPAKEEAPAPTPAASAPAPAAAAPAPVAAAAPAAAAAEIADEPVKASLLLHVLVAHKLKKSLDSIPMSKTIKDLVGGKSTVQNEILGDLGKEFGTTPEKPEETPLEELAETFQDTFSGALGKQSSSLLSRLISSKMPGGFTITVARKYLQTRWGLPSGRQDGVLLVALSNEPAARLGSEADAKAFLDSMAQKYASIVGVDLSSAASASGAAGAGAAAGAAMIDAGALEEITKDHKVLARQQLQVLARYLKMDLDNGERKFLKEKDTVAELQAQLDYLNAELGEFFVNGVATSFSRKKARTFDSSWNWAKQSLLSLYFEIIHGVLKNVDREVVSEAINIMNRSNDALIKFMEYHISNTDETKGENYQLVKTLGEQLIENCKQVLDVDPVYKDVAKPTGPKTAIDKNGNITYSEEPREKVRKLSQYVQEMALGGPITKESQPTIEEDLTRVYKAISAQADKQDISSSTRVEFEKLYSDLMKFLESSKEIDPSQTTQLAGMDVEDALDKDSTKEVASLPNKSTISKTVSSTIPRETIPFLHLRKKTPAGDWKYDRQLSSLFLDGLEKAAFNGVTFKDKYVLITGAGKGSIGAEVLQGLLQGGAKVVVTTSRFSKQVTDYYQSIYAKYGAKGSTLIVVPFNQGSKQDVEALIEFIYDTEKNGGLGWDLDAIIPFAAIPEQGIELEHIDSKSEFAHRIMLTNILRMMGCVKKQKSARGIETRPAQVILPMSPNHGTFGGDGMYSESKLSLETLFNRWHSESWANQLTVCGAIIGWTRGTGLMSANNIIAEGIEKMGVRTFSQKEMAFNLLGLLTPEVVELCQKSPVMADLNGGLQFVPELKEFTAKLRKELVETSEVRKAVSIETALEHKVVNGNSADAAYAQVEIQPRANIQLDFPELKPYKQVKQIAPAELEGLLDLERVIVVTGFAEVGPWGSARTRWEMEAFGEFSLEGCVEMAWIMGFISYHNGNLKGRPYTGWVDSKTKEPVDDKDVKAKYETSILEHSGIRLIEPELFNGYNPEKKEMIQEVIVEEDLEPFEASKETAEQFKHQHGDKVDIFEIPETGEYSVKLLKGATLYIPKALRFDRLVAGQIPTGWNAKTYGISDDIISQVDPITLFVLVSVVEAFIASGITDPYEMYKYVHVSEVGNCSGSGMGGVSALRGMFKDRFKDEPVQNDILQESFINTMSAWVNMLLISSSGPIKTPVGACATSVESVDIGVETILSGKARICIVGGYDDFQEEGSFEFGNMKATSNTLEEFEHGRTPAEMSRPATTTRNGFMEAQGAGIQIIMQADLALKMGVPIYGIVAMAATATDKIGRSVPAPGKGILTTAREHHSSVKYASPNLNMKYRKRQLVTREAQIKDWVENELEALKLEAEEIPSEDQNEFLLERTREIHNEAESQLRAAQQQWGNDFYKRDPRIAPLRGALATYGLTIDDLGVASFHGTSTKANDKNESATINEMMKHLGRSEGNPVIGVFQKFLTGHPKGAAGAWMMNGALQILNSGIIPGNRNADNVDKILEQFEYVLYPSKTLKTDGVRAVSITSFGFGQKGGQAIVVHPDYLYGAITEDRYNEYVAKVSAREKSAYKFFHNGMIYNKLFVSKEHAPYTDELEEDVYLDPLARVSKDKKSGSLTFNSKNIQSKDSYINANTIETAKMIENMTKEKVSNGGVGVDVELITSINVENDTFIERNFTPQEIEYCSAQPSVQSSFAGTWSAKEAVFKSLGVKSLGGGAALKDIEIVRVNKNAPAVELHGNAKKAAEEAGVTDVKVSISHDDLQAVAVAVSTKK

SEQ ID NO: 20 - protein, 2037 aa, Candida albicans
MSTHRPFQLTHGSIEHTLLVPNDLFFNYSQLKDEFIKTLPEPTEGFAGDDEPSSPAELYGKFIGFISNAQFPQIVELSLKDFESRFLDNNNDNIHSFAVKLLDDETYPTTIAKVKENIVKNYYKAVKSINKVESNLLYHCKHDAKLVAIFGGQGNTDDYFEELRELYTLYQGLIEDLLVSIAEKLNQLHPSFDKIYTQGLNILSWLKHPETTPDQDYLLSVPVSCPVICVIQLCHYTITCKVLGLTPGEFRNSLKWSTGHSQGLVTAVTIAASDSWDSFLKNSLTAVSLLLFIGSRCLSTYPRTSLPPTMLQDSLDNGEGRPSPMLSVRDLSIKQVEKFIEQTNSHLPREKHIAISLINGARNLVLSGPPESLYGFNLNLRNQKAPMGLDQSRVPFSERKLKCSNRFLPIFAPFHSHLLADATELILDDVKEHGLSFEGLKIPVYDTFDGSDFQALKEPIIDRVVKLITELPVHWEEATNHKATHILDFGPGGVSGLGVLTHRNKEGTGARIILAGTLDSNPIDDEYGFKHEIFQTSADKAIKWAPDWLKELRPTLVKNSEGKIYVKTKFSQLLGRAPLMVAGMTPTTVNTDIVSASLNAGYHIELAGGGYFSPVMMTRAIDDIVSRIKPGYGLGINLIYVNPFMLQWGIPLIKDLREKGYPIQSLTIGAGVPSIEVATEYIEDLGLTHLGLKPGSVDAISQVIAIAKAHPTFPIVLQWTGGRGGGHHSFEDFHQPIIQMYSKIRRCSNIVLVAGSGFGSDEDTYPYLSGYWSEKFNYPPMPFDGVLFGSRVMTSKESHTSLAAKKLIVECKGVPDQQWEQTYKKPTGGIITVRSEMGEPIHKIATRGVMFWKELDDTIFNLPKNKLLDALNKKRDHIIKKLNNDFQKPWFGKNANGVCDLQEMTYKEVANRLVELMYVKKSHRWIDVSLRNMYGDFLRRVEERFTSSAGTVSLLQNFNQLNEPEQFTADFFEKFPQAGKQLISEEDCDYFLMLAARPGQKPVPFVPVLDERFEFFFKKDSLWQSEDLESVVDEDVQRTCILHGPVASQYTSKVDEPIGDILNSIHEGHIARLIKEEYAGDESKIPVVEYFGGKKPASVSATSVNIIDGNQVVYEIDSELPNKQEWLDLLAGTELNWLQAFISTDRIVQGSKHVSNPLHDILTPAKHSKVTIDKKTKKLTAFENIKGDLLPVVEIELVKPNTIQLSLIEHRTADTNPVALPFLYKYNPADGFAPILEIMEDRNERIKEFYWKLWFGSSVPYSNDINVEKAILGDEITISSQTISEFTHAIGNKCDAFVDRPGKATLAPMDFAIVIGWKAIIKAIFPKSVDGDLLKLVHLSNGYKMITGAAPLKKGDVVSTKAEIKAVLNQPSGKLVEVVGTIYREGKPVMEVTSQFLYRGEYNDYCNTFQKVTETPVQVAFKSAKDLAVLRSKEWFHLEKDVQFDVLTFRCESTYKFKSANVYSSIKTTGQVLLELPTKEVIQVGSVDYEAGTSYGNPVTDYLSRNGKTIEESVIFENAIPLSSGEELTSKAPGTNEPYAIVSGDYNPIHVSRVFAAYAKLPGTITHGMYSSASIRALVEEWAANNVAARVRAFKCDFVGMVLPNDTLQTTMEHVGMINGRKIIKVETRNVETELPVLIGEAEIEQPTTTYVFTGQGSQEQGMGMELYNSSEVAREVWDKADRHFVNNYGFSILDIVQNNPNELTIHFGGAKGRAIRDNYIGMMFETIGEDGALKSEKIFKDIDETTTSYTFVSPTGLLSATQFTQPALTLMEKAAYEDIKSKGLIPSDIMFAGHSLGEYSALSSLANVMPIESLVDVVFYRGMTMQVAVPRDELGRSNYGMVAVNPSRVSATFDDSALRFVVDEVANKTKWLLEIVNYNVENQQYVAAGDLRALDTLTNVLNVLKINKIDIVKLQEQMSIEKVKEHLYEIVDEVAAKSLAKPQPIDLERGFAVIPLKGISVPFHSSYLMSGVKPFQRFLCKKIPKSSVKPQDLIGKYIPNLTAKPFELTKEYFQSVYDLTKSEKIKSILDNWEQYE

SEQ ID NO: 21 - DNA, 6072 bp, Candida albicans
ggatccttttttttttgggtaatattaacaatccagcttaggccatattgttgggtgtccttaaaaattatgtgccaattatttacttatatattgatatagctctccttttctcttttttatatttttcaaagttttttttattcttttactgtttattcaactaacttgtttttatttctcccccaattaacaatgaaaccagaaattgaacaagaattatcccacactttgttaactgaattgttggcatatcaatttgcttctccagttagatggattgaaactcaagatgtctttttaaaacagcataatactgaaagaatcatcgaaattggtccttcaccaactttagctggtatggccaatagaactatcaaagccaaatatgaatcctatgatgctgctttatctttgcaacgacaagtcttgtgttactccaaagatgctaaggagatttactacaagccagatccagcagatcttgctcctaaggaaacaccaaagcaagaagagagtaccccatcagctcctgccgctgccactccaacacctgctgctgccgctgctcctactccagcaccagctcctgcaagtgctggcccagttgaatctattccagatgaaccagtcaaggctaacttgttaatccatgttttggttgcacaaaaattaaagaaacctttagatgctgttccaatgaccaaggcaattaaggatttggttaatggtaaatccactgttcaaaatgaaattcttggtgacttgggtaaggaatttggctctactcctgaaaaaccggaagacactccattggaagaattagctgaacaattccaagattcattcagcggtcaattaggaaagacttctacttcattgattggtagattaatgtcctcaaagatgccgggtggattttccatcactactgctagaaagtatttggaatcaagatttggtttgggtgctggtagacaagattctgtcttgttgatggctttaacaaatgaaccagctaatagattaggttctgaagccgatgcaaaaactttctttgatggaattgctcaaaaatacgcatcaagtgctgggatctccttgtcatcaggagcaggctccggtgcaggcgccgcaaatagtggtggtgctgttgttgatagtgctgccttagatgctttaacagctgaaaacaagaaattagccaaacagcaattagaagttttagcaagatacttgcaaagtcgacttaaacaagggagccttaaatcttttatcaaggaaaaggaagcttctgctgttttacaaaaagagttagatttgtgggaagcagaacacggagaattctatgctaagggtatccaaccaactttctccgcattaaagtctagaacttatgactcctattggaattgggcccgtcaagacgttttatcaatgtatttcgacattatttttggcaagttaacttctgttgatagagaaaccatcaaccaatgtattcaaatcatgaacagagccaatccaactttaatcaagtttatgcaatatcatatcgaccattgtccagaatataaaggtgaaacttataaattggccaagagattgggtcaacaattgattgacaactgtaaacaagttttgactgaagatccagtttacaaagatgtttccagaattactggtccaaagactaaagtcagtgctaagggtaacattgaatatgaggaaactcaaaaggattcagttagaaaatttgaacaatatgtgtatgaaatggcccaaggtggtgctatgaccaaagttagtcaaccaactattcaagaagatttagctagagtttacaaggctatttccaaacaagcttccaaagatagcaaattggaattgcaaagagtttacgaagatttattgaaggtggttgaaagttccaaggaaatcgaaaccgaacaattgactaaagatattttacaagctgctacagttccaacaaccccaacagaggaagtagacgatccttgtactccttcttcggatgatgaaattgcttctttaccagataagacttctatcattcaacctgtctcgtctactattccatctcaaactattccatttttgcacattcagaaaaagaccaaagacggttgggaatacaataagaaattatcttctctttacttggatggattggaatcagctgccattaatggtttaactttcaaagacaagtatgtcttagttactggtgctggtgctggctctattggtgccgaaattttgcaaggtttaatcagtggtggtgccaaagttattgtcacaacctctagattttccaagaaagttaccgagtattatcaaaacatgtatgccagatatggtgctgctgggtctactttaattgttgttccgttcaaccaaggttctaaacaagatgttgatgcattggttcaatacatttatgatgagccaaagaaaggtggtttgggttgggatttggatgcaatcattccatttgctgctattccagaaaatggtaatggtctcgacaacattgattctaaatctgaatttgcccacagaatcatgttgaccaaccttttaagattgttaggtgctgttaaatccaaaaagcccactgacactagacctgctcaatgtattttgccattatctccaaatcacggaacttttggttttgacgggttgtactctgaatctaaaatctcattggaaaccttattcaacagatggtattctgaagattggggatccaagttgactgtttgtggtgccgtaattgggtggactagaggtacaggtttgatgagtgccaataacattattgctgaaggtattgaaaaattgggtgtcagaactttctcccaaaaggaaatggctttcaatattttaggtttattgacaccagaaattgtacaattatgtcaagaagaaccagttatggctgacttgaatggtggtttgcaattcattgacaacttgaaggatttcacatctaaattaagaaccgacttgttggaaactgcagacattagaagagctgtttctattgaatcagctatcgagcaaaaagttgtcaatggtgacaatgtcgatgcaaactactcaaaggttatggttgaacctagagccaacatgaaatttgatttcccaactttgaaatcttatgatgaaatcaaacaaattgctccagaattggaaggtatgttggatttggaaaatgttgtcgttgtgacaggttttgctgaagttggtccatggggtaactctagaaccagatgggaaatggaagcttatggtgagttctcattggaaggtgccattgaaatggcttggattatgggtttcatcaagtatcataatggtaatttgcaagggaaaccatactctggatgggttgatgccaagactcaaactccaattgacgaaaaggatatcaaatccaaatatgaagaagaaattttagaacattccggtattagattgattgagccagaattgttcaatggctatgatccaaagaaaaaacaaatgattcaagaaattgttgttcaacacgatttagaaccatttgaatgttctaaagaaacagctgagcaatacaaacacgaacacggagaaaaatgtgaaatttttgaaattgaagaaagtggtgaatacacagttagaatcttgaaaggtgcaacattgtacgttccgaaagctttgagatttgatagattagttgctggtcaaattccaactggttgggacgctcgtacctatggtatcccagaagacactattagtcaagttgatccaatcactttgtacgtgttggttgccactgttgaagccttgttgtctgctggtattactgatccatatgaattctacaaatacgttcatgtgtctgaagttggtaactgttctggttccggtatgggaggtgtctctgctttgagaggaatgttcaaagatagatatgctgacaaaccagttcaaaatgacattttgcaagaatcatttatcaacactatgtctgcttgggtcaatatgttgttgttgtcttcctctggtccaatcaagacaccagtcggtgcttgtgccactgctgttgaatcggttgacattggtattgaaacaattttgtctggtaaagctaaagtagttttggtaggtggttacgatgacttccaagaagaagggtcttatgaattcgccaatatgaatgctacttctaattctattgaagagttcaaacacggaagaacaccaaaggaaatgtcaagaccaactactactaccagaaatggtttcatggaagctcaaggttctggtattcaagttatcatgactgctgatttggctctcaagatgggtgttccaatccacgctgtattggccatgactgctactgccactgataagattggtagatctgttccagcaccaggtaaaggtattttgaccactgccagagaacatcatggcaacttgaagtacccatctccacttttgaacatcaagtacaggaagagacaattgaacaaaagattggaacaaatcaaatcttgggaagaaacagaactttcttacttgcaagaagaagccgagttggccaaagaagaatttggtgacgaattttctatgcatgagttcttgaaagagagaactgaagaagtgtaccgtgaatcaaagagacaagtttctgatgctaagaaacaatggggtaattcattctacaagtctgatccaagaattgctccattgagaggagcattggctgccttcaacttaaccatcgatgatattggtgttgcatccttccatggtacttccaccgttgctaacgataagaatgaatctgccacaatcaacaatatgatgaaacacttgggtagatccgaaggtaacccagtatttggtgttttccaaaaatacttgacaggtcatccaaaaggtgcagctggtgcttggatgttgaatggtgccattcaaattcttgagtctggtcttgttccaggtaacagaaatgcggataatgttgataagcttttagaacaatacgaatatgtattgtacccatcaagatcaattcaaaccgatggtattaaagccgtttctgttacatcatttggtttcggtcaaaaaggtgcacaagccgttgttgttcatccagattacttatttgctgttttggatagatccacttatgaagaatatgctactaaggtctctgctagaaataaaaagacctaccgttacatgcacaatgcaatcaccagaaacactatgtttgttgccaaagacaaagctccatatagtgacgaattggaacaaccagtttacttggatccattggctcgtgttgaagaaaacaagaaaaagttggtattcagtgacaaaacaattcaatcgaaccaatcttatgttggagaagttgctcaaaaaactgctaaggcattgtctactttaaacaaatcatcaaagggagttggtgtagatgttgaattgttgtcagcaatcaatatcgacaatgaaacctttattgaaagaaactttactggtaatgaagttgaatactgtttgaatactgctcacccacaagcttcattcactggaacttggtcagcaaaggaagctgttttcaaagccttgggtgttgaatcaaaaggtgctggagcaagcttgattgatattgaaatcactcgtgacgttaatggtgctcctaaagtaattttgcatggtgaggccaaaaaagctgctgctaaagctggtgttaaaaatgtcaatatttcaatttctcatgatgatttccaagctactgctgttgctttaagtgaattttaaaattagtagtgtttagaaatattcgtgtatatctgatcaaaaacttttttgatttttaatatatgtccggttgtacaatttttttttctgttgatttaaactgatctcattattttgttctctcacagctcacagcctacaaccataaaaaaagcccaacactcacttttgctcactggttcaccaccactacggaaaaaataagaacaacaaataaaa

SEQ ID NO: 22 - protein, 1885 aa, Candida albicans
MKPEIEQELSHTLLTELLAYQFASPVRWIETQDVFLKQHNTERIIEIGPSPTLAGMANRTIKAKYESYDAALSLQRQVLCYSKDAKEIYYKPDPADLAPKETPKQEESTPSAPAAATPTPAAAAAPTPAPAPASAGPVESIPDEPVKANLLIHVLVAQKLKKPLDAVPMTKAIKDLVNGKSTVQNEILGDLGKEFGSTPEKPEDTPLEELAEQFQDSFSGQLGKTSTSLIGRLMSSKMPGGFSITTARKYLESRFGLGAGRQDSVLLMALTNEPANRLGSEADAKTFFDGIAQKYASSAGISLSSGAGSGAGAANSGGAVVDSAALDALTAENKKLAKQQLEVLARYLQSRLKQGSLKSFIKEKEASAVLQKELDLWEAEHGEFYAKGIQPTFSALKSRTYDSYWNWARQDVLSMYFDIIFGKLTSVDRETINQCIQIMNRANPTLIKFMQYHIDHCPEYKGETYKLAKRLGQQLIDNCKQVLTEDPVYKDVSRITGPKTKVSAKGNIEYEETQKDSVRKFEQYVYEMAQGGAMTKVSQPTIQEDLARVYKAISKQASKDSKLELQRVYEDLLKVVESSKEIETEQLTKDILQAATVPTTPTEEVDDPCTPSSDDEIASLPDKTSIIQPVSSTIPSQTIPFLHIQKKTKDGWEYNKKLSSLYLDGLESAAINGLTFKDKYVLVTGAGAGSIGAEILQGLISGGAKVIVTTSRFSKKVTEYYQNMYARYGAAGSTLIVVPFNQGSKQDVDALVQYIYDEPKKGGLGWDLDAIIPFAAIPENGNGLDNIDSKSEFAHRIMLTNLLRLLGAVKSKKPTDTRPAQCILPLSPNHGTFGFDGLYSESKISLETLFNRWYSEDWGSKLTVCGAVIGWTRGTGLMSANNIIAEGIEKLGVRTFSQKEMAFNILGLLTPEIVQLCQEEPVMADLNGGLQFIDNLKDFTSKLRTDLLETADIRRAVSIESAIEQKVVNGDNVDANYSKVMVEPRANMKFDFPTLKSYDEIKQIAPELEGMLDLENVVVVTGFAEVGPWGNSRTRWEMEAYGEFSLEGAIEMAWIMGFIKYHNGNLQGKPYSGWVDAKTQTPIDEKDIKSKYEEEILEHSGIRLIEPELFNGYDPKKKQMIQEIVVQHDLEPFECSKETAEQYKHEHGEKCEIFEIEESGEYTVRILKGATLYVPKALRFDRLVAGQIPTGWDARTYGIPEDTISQVDPITLYVLVATVEALLSAGITDPYEFYKYVHVSEVGNCSGSGMGGVSALRGMFKDRYADKPVQNDILQESFINTMSAWVNMLLLSSSGPIKTPVGACATAVESVDIGIETILSGKAKVVLVGGYDDFQEEGSYEFANMNATSNSIEEFKHGRTPKEMSRPTTTTRNGFMEAQGSGIQVIMTADLALKMGVPIHAVLAMTATATDKIGRSVPAPGKGILTTAREHHGNLKYPSPLLNIKYRKRQLNKRLEQIKSWEETELSYLQEEAELAKEEFGDEFSMHEFLKERTEEVYRESKRQVSDAKKQWGNSFYKSDPRIAPLRGALAAFNLTIDDIGVASFHGTSTVANDKNESATINNMMKHLGRSEGNPVFGVFQKYLTGHPKGAAGAWMLNGAIQILESGLVPGNRNADNVDKLLEQYEYVLYPSRSIQTDGIKAVSVTSFGFGQKGAQAVVVHPDYLFAVLDRSTYEEYATKVSARNKKTYRYMHNAITRNTMFVAKDKAPYSDELEQPVYLDPLARVEENKKKLVFSDKTIQSNQSYVGEVAQKTAKALSTLNKSSKGVGVDVELLSAINIDNETFIERNFTGNEVEYCLNTAHPQASFTGTWSAKEAVFKALGVESKGAGASLIDIEITRDVNGAPKVILHGEAKKAAAKAGVKNVNISISHDDFQATAVALSEF

SEQ ID NO: 23 - protein, 3069 aa, Mycobacterium tuberculosis
MTIHEHDRVSADRGGDSPHTTHALVDRLMAGEPYAVAFGGQGSAWLETLEELVSATGIETELATLVGEAELLLDPVTDELIVVRPIGFEPLQWVRALAAEDPVPSDKHLTSAAVSVPGVLLTQIAATRALARQGMDLVATPPVAMAGHSQGVLAVEALKAGGARDVELFALAQLIGAAGTLVARRRGISVLGDRPPMVSVTNADPERIGRLLDEFAQDVRTVLPPVLSIRNGRRAVVITGTPEQLSRFELYCRQISEKEEADRKNKVRGGDVFSPVFEPVQVEVGFHTPRLSDGIDIVAGWAEKAGLDVALARELADAILIRKVDWVDEITRVHAAGARWILDLGPGDILTRLTAPVIRGLGIGIVPAATRGGQRNLFTVGATPEVARAWSSYAPTVVRLPDGRVKLSTKFTRLTGRSPILLAGMTPTTVDAKIVAAAANAGHWAELAGGGQVTEEIFGNRIEQMAGLLEPGRTYQFNALFLDPYLWKLQVGGKRLVQKARQSGAAIDGVVISAGIPDLDEAVELIDELGDIGISHVVFKPGTIEQIRSVIRIATEVPTKPVIMHVEGGRAGGHHSWEDLDDLLLATYSELRSRANITVCVGGGIGTPRRAAEYLSGRWAQAYGFPLMPIDGILVGTAAMATKESTTSPSVKRMLVDTQGTDQWISAGKAQGGMASSRSQLGADIHEIDNSASRCGRLLDEVAGDAEAVAERRDEIIAAMAKTAKPYFGDVADMTYLQWLRRYVELAIGEGNSTADTASVGSPWLADTWRDRFEQMLQRAEARLHPQDFGPIQTLFTDAGLLDNPQQAIAALLARYPDAETVQLHPADVPFFVTLCKTLGKPVNFVPVIDQDVRRWWRSDSLWQAHDARYDADAVCIIPGTASVAGITRMDEPVGELLDRFEQAAIDEVLGAGVEPKDVASRRLGRADVAGPLAVVLDAPDVRWAGRTVTNPVHRIADPAEWQVHDGPENPRATHSSTGARLQTHGDDVALSVPVSGTWVDIRFTLPANTVDGGTPVIATEDATSAMRTVLAIAAGVDSPEFLPAVANGTATLTVDWHPERVADHTGVTATFGEPLAPSLTNVPDALVGPCWPAVFAAIGSAVTDTGEPVVEGLLSLVHLDHAARVVGQLPTVPAQLTVTATAANATDTDMGRVVPVSVVVTGADGAVIATLEERFAILGRTGSAELADPARAGGAVSANATDTPRRRRRDVTITAPVDMRPFAVVSGDHNPIHTDRAAALLAGLESPIVHGMWLSAAAQHAVTATDGQARPPARLVGWTARFLGMVRPGDEVDFRVERVGIDQGAEIVDVAARVGSDLVMSASARLAAPKTVYAFPGQGIQHKGMGMEVRARSKAARKVWDTADKFTRDTLGFSVLHVVRDNPTSIIASGVHYHHPDGVLYLTQFTQVAMATVAAAQVAEMREQGAFVEGAIACGHSVGEYTALACVTGIYQLEALLEMVFHRGSKMHDIVPRDELGRSNYRLAAIRPSQIDLDDADVPAFVAGIAESTGEFLEIVNFNLRGSQYAIAGTVRGLEALEAEVERRRELTGGRRSFILVPGIDVPFHSRVLRVGVAEFRRSLDRVMPRDADPDLIIGRYIPNLVPRLFTLDRDFIQEIRDLVPAEPLDEILADYDTWLRERPREMARTVFIELLAWQFASPVRWIETQDLLFIEEAAGGLGVERFVEIGVKSSPTVAGLATNTLKLPEYAHSTVEVLNAERDAAVLFATDTDPEPEPEEDEPVAESPAPDVVSEAAPVAPAASSAGPRPDDLVFDAADATLALIALSAKMRIDQIEELDSIESITDGASSRRNQLLVDLGSELNLGAIDGAAESDLAGLRSQVTKLARTYKPYGPVLSDAINDQLRTVLGPSGKRPGAIAERVKKTWELGEGWAKHVTVEVALGTREGSSVRGGAMGHLHEGALADAASVDKVIDAAVASVAARQGVSVALPSAGSGGGATIDAAALSEFTDQITGREGVLASAARLVLGQLGLDDPVNALPAAPDSELIDLVTAELGADWPRLVAPVFDPKKAVVFDDRWASAREDLVKLWLTDEGDIDADWPRLAERFEGAGHVVATQATWWQGKSLAAGRQIHASLYGRIAAGAENPEPGRYGGEVAVVTGASKGSIAASVVARLLDGGATVIATTSKLDEERLAFYRTLYRDHARYGAALWLVAANMASYSDVDALVEWIGTEQTESLGPQSIHIKDAQTPTLLFPFAAPRVVGDLSEAGSRAEMEMKVLLWAVQRLIGGLSTIGAERDIASRLHVVLPGSPNRGMFGGDGAYGEAKSALDAVVSRWHAESSWAARVSLAHALIGWTRGTGLMGHNDAIVAAVEEAGVTTYSTDEMAALLLDLCDAESKVAAARSPIKADLTGGLAEANLDMAELAAKAREQMSAAAAVDEDAEAPGAIAALPSPPRGFTPAPPPQWDDLDVDPADLVVIVGGAEIGPYGSSRTRFEMEVENELSAAGVLELAWTTGLIRWEDDPQPGWYDTESGEMVDESELVQRYHDAVVQRVGIREFVDDGAIDPDHASPLLVSVFLEKDFAFVVSSEADARAFVEFDPEHTVIRPVPDSTDWQVIRKAGTEIRVPRKTKLSRVVGGQIPTGFDPTVWGISADMAGSIDRLAVWNMVATVDAFLSSGFSPAEVMRYVHPSLVANTQGTGMGGGTSMQTMYHGNLLGRNKPNDIFQEVLPNIIAAHVVQSYVGSYGAMIHPVAACATAAVSVEEGVDKIRLGKAQLVVAGGLDDLTLEGIIGFGDMAATADTSMMCGRGIHDSKFSRPNDRRRLGFVEAQGGGTILLARGDLALRMGLPVLAVVAFAQSFGDGVHTSIPAPGLGALGAGRGGKDSPLARALAKLGVAADDVAVISKHDTSTLANDPNETELHERLADALGRSEGAPLFVVSQKSLTGHAKGGAAVFQMMGLCQILRDGVIPPNRSLDCVDDELAGSAHFVWVRDTLRLGGKFPLKAGMLTSLGFGHVSGLVALVHPQAFIASLDPAQRADYQRRADARLLAGQRRLASAIAGGAPMYQRPGDRRFDHHAPERPQEASMLLNPAARLGDGEAYIG

SEQ ID NO: 24 - protein, 3076 aa, Mycobacterium tuberculosis
MTIHEHDQVSADRNGNSLHGSRALADRLKAGEPYVVAFGGQGSAWLETLEELVSSAGLEADLATLVCEVELLLEPVAKELVVVRPIGFEPLQWVRALLAEDLVPSDKHLTSAAVSVPGVLLTQIAVGRALARQGMDLIATPPVGIVGHSQGVLAVEALKAGGARDAELLAMAQLIGAAGTLVARRRGISVLGDRPPMVSVTNADPERIRRLLDEFAQDVRTVLPPVLSIRNGWRSVVITGTPEQLSRFERYCRQISDKEEEDRRKKIRGGDIFAPVFDPVQVEIGFHTPHLADGIGIVGGWAEKVGLDVTLARELTEAILVRGVDWVREITRVHGAGVRWIIDLGPGDILTRLTAPVIRGLGVGIVPVANRGGQRTLFTVGAVPEVVRAWLSYAPTVVQLPDGRIKLSTKFTRLTGRSPILLAGMTPTTVDANIVAAAANAGHWAELAGGGQVTEEIFANRVEQLSGLLEPGRTYQFNALFLDPYLWKLQVGGKRLVQKARQSGAAIDGVVISGGILDLEDAVELIEELGGIGISYVVFKPGTIEQIRSVIRIATEMSTKPVIMHVEGGRAGGHHSWEDLDDLLLATYSELRSHANITVCVGGGIGTPEKAAEYLSGRWAQAYGFPLMPIDGILVGTAAMATKEATTSPSVKRMLVETQGTDQWIGSGKAQGGMASSRSQLGADIHEIDNAASRCGRLLDEVAGDAEAVAERRDEIIAAMANTAKPYFGDVSEMTYLQWLQRYVELTIGEGNSTADTASPGSPWLADTWRDRFQKMLQRAESRLHPSDFGLIKTIFTDPVLLEKPNQAIAALLKYYPDAETVQLHPADAPFFVMLCQMLGKPVNFVPVIDKDVRRWWRSDSLWQAHDARYDADQVCIIPGIAAVAGITQMDEPVGELLDRFEQAAIDEVLAGGAEPVVVMSRRLGRADVAGPLAVVLDAPDVLWAGRIATNPVHRIADPNEWQVNGNLSATHSSTGAQLQVKSEDQQVVLSVPVSNGWIDIPFTLPTNTVDGGALLVSTEDATSAMRAVLAIVAGVDGPELLSPVKDGTAIVTVDWNPERVADHTGVTATFREPLAPSLATVPDALVGACWPAVFSAIGSAVTEAGVLVVEGLLNLLHLDHAVCVVGKLPTVPAQLTVTATVSLAIDTDMGRVVPVSVTIRDTTGADGAVLATLEERFVILGRTGTAELTGPVRAGGAISENATDTPRRRRRDVTLTAPIDMRPFAVVSGDHNPIHTDRTAALLAGLESPIVHGMWLSAAAQHVVMATDGQARPAARLIGWTARFLGMAHPGDKVDFRVDRIGIDQGAEILEVSARISSGLVMSATARLAAPKTVYAFPGQGIQHKGMGMDVRARSKAARRVWDDADKFTRSGLGFSVLHVVRDNPTNITANGVHYHHPDGVLYLTQFTQVAMATVAVAQVAEMREQGAFVEGAIACGHSVGEYTALACVMGVYELEALLETVFHRGSKMHDIVLRDELGRSNYRLAAIRPSQIGLPDDEVPAFVRGIAESTGEFLEIVNFNLRGSQYAIAGTVHGLEALEAEVERRRELTGGRRSFILVPGIDVPFHSRVLRVGVAEFRRSLDRVLPQDQDPDWIIGRYIPNLVPRPFTLARDFIQEIRDLVPAEPLDDILADYDTWRRERPSEMARRVLIELLAWQFASPVRWIETQDLLFTEEAAGGLGVERFVEIGVKSAPTVAGLATDTLKLPEYSHNTVEVLNVERDAAVLFATDTDPELEPEPENVSDASAALPAESALALGTVAPAPVVPSGPRPEDISFGAADATLALIALSAKMRLDQIEEMDSIESITDGASSRRNQLLVDLGSELSLGAIDGVAEADLAGLRSQVTKLARTYKPYGPVLSELINDQLRSALGPSGKRPGVIAERVKKIWELGDGWVKHVTVEIALGTREGTSVRGGPLGNLNEGALADVDSVDKAVDAAVASVAARHGVVVALPSAGSGGSATVDVAALSEFTDQITGHDGVLASAARLVLGQLGLDGPVTAAPATTDTGLIDLVTAELSTDWPRLVAPVFDVKKAVVFDDRWASAREDLVRLWLNDEGEIEAQWSHLSERFEGAGHVVATQATWWQGKSLAAGRQIHASLYGRIAAGAQNPDRGLYSSEIAVVTGASKGSIAASVAARLLDGGATVIATTSKLDEERITFYRALYRDHARYGAALWVVAANMASYSDIDALVEWIGNEQTESLGPQSIHIKDAQTPTLLFPFAAPRVIGDLSEAGARSEIEMKVLLWAVQRLIVGLSKIGTERDVASRLHVVLPGSPNRGMFGGDGAYGEAKSALDAVVSRWHAESSWAARVSLAHALIGWTRGTGLMGHNDVIVSAVEEAGVTTYSTDEMAAMLLDLCNAESKVAAAGTPITVDLTGGLGEVDLDMAELAAKAREDHAAQAAEDEATEASEVAGTIAALPSPPRGYTPASPHWDDLDVDPADLVVIVGGAEIGPYGSSRTRFEMEVAGELSAAGVLELVWTTGLIRWEDDPQPGWYDTESGELVDESELVERYHDTVVQRCGIREFVDDGTIDPDHAYPLLVSVFLDKDFAFVVSSEADARAFVEFDPEHTVIRPVPDSSDWQVIRKAGTEIRVPRKMKLSRVVGGQIPTGFDPTVWGISPDMVSSIDRVAVWSIVATVDAFLSAGFTPAEVMRYVHPSLVANTMGTGMGGGTSIQRLYHSSLLGRNKPNDIFQEILPNIVAAHVVQSYIGSYGSMIHPVAACATAAVSVEEGVDKIRLGKAELVVAGGIDDLTLEGIIGFGDMAATADTAMMRGRGIHDSKFSRPNDRRRLGFVEAQGGGTILLARGDLALKMGLPVFAVVAFAQSFGDGVHTSIPAPGLGALGAGRGGKDSPLVQSLAKLGVSADDIAVISKHDTSTLANDPNETELHERLADAMGRSAGAPLFVVSQKSLTGHAKGGAAVFQMMGLCQMLRDGVIPPNRSLDCVDEELAGAAHFVWLRDTLRLGEKFPLKAGMLTSLGFGHVSGLVALVHPQAFIAALDPGQRDDYQRRANVRLLAGQRRLASAIAGGAPMYERPPDRRFDHHVPEKLQEAAMLLNPAARLGDGDAYIG

SEQ ID NO: 25 - protein, 2586 aa, Caenorhabditis elegans
MDPTQWWQKQDDIVISGVSGRFPRCDNVKMFGDMLLAGEDLVTEDSLRWTPGFCDLPKRHGKLKVLNKFDAGFFQVTPKQANFMDPQVRLLLEASWEAMVDAGINPTDLRGSKTGVFVGCSASETSGMLTQDPDTVTGYTLTGCVRSMFSNRISYTFDLQGPSFSVDTACSSSLLALQLAVDSIRQGQCDAAIVAGAHLTLTPTAALQFLRLGMLTDKGSCRSFDESGDGYCRTEGVAAIFIQRKKKAQRLYATVVHAKSNTDGHKEHGITFPSGERQAQLLQEVYSEAGIDPNSVYYVEAHGTGTKVGDPQEANAICEVFCSKRTDSLLIGSVKSNMGHAEPASGVCSLTKILLSIERQLIPPNLHYNTPNQYIPGLTDGRLKVVTEPTALPGGLIGINSFGFGGSNTHVILKAADHIAPPITPHPFTKLVTYCGRTQEAVENIFTEIESNKDDLYLQALLANQANMPANLLPFRGYMLLDRENNVETLKSITKVPITEARPIYFIYSGMGSQWPGMAIKLMKIPMFDDSLRASSKTLEEFGLDVYGMLCNPDPEQYSNNTMNCMLAITAIQIALTDVLTALGVSPDGIIGHSTGEMGCGYADGGITREQTMRLAYHRGTTIMKHTEIKGAMAAVGLTWEQVKEQAPPGVVAACHNGADSVTISGDAEGVATFCAQLKEKDIFAKVVDTSGIPFHSPAMLAVQDEMIECMRTAVPEPKPRSSKWISTSIPEDDWESDLAATCSAEYHVHNACSPVLFYEAIQKIPANAVTIEMAPHSLMQAILRRSLQKTVTNVGLMNRPKSENDDELESFLGSLGKIYQAGVNIQITELYPGGQYKGVVPKGTPMIGPMWKWDHTQDWLTIDGRQVLAGGSGSVASSATYNIDPFATDSKETYLLDHVIDGRVLYPFTGHMVLAWRTLCKLKGLDYTKTPVVFENINVFSATILTKPIKLDVVLSPGNGYFEIISDDQVAASGRIYIPEDNQPFYYGKLEDIRTSEIADRIELDTEDAYKEFLLRGYEYGQAFRGIYKTCNSGERGFLYWTGNWVTFLDSLLQTALLAERSDTLRLPTRVRHLRIDPNKHLEHVVEKDGIQVIELRNDHSTNGCIAGGVECCDLNAHSVARRIQVSGQLYHEKIFFVPHFDHNCLSGHKKTSTILKDYSAVIKQQLYTGFSKWQSAGLLKKLKNGAQIVKALAVLKASQSDVVLDDTVTRFTHDGKCTVLHHIADMFKIEDCEDFEDRVAAKLKSVRGIFELDRLWAGAVLNDRIVKSLQDICIENSAGHHATMAAVDLVSTDQIRHCIEANSSHPLLETDYTCIGANVDHLDESTLEIIGGKKQKIDLENNFTGHGEVKNLDYVLLDKVISKKADPIAFIEACKHLIRETGFLLVVEVTSQYEIALAIEGLLGNEMVGDASRKYNQFFTHEQLLDMFKSTGFLICNFQSDPALMTTTYAVRRVSPIPRDPVFIDVDDVKEFNWIEPLQKVSEERLNEPDSKTIWLVSNKCRNNGIVGLGLCFVEENLKINRFRSAFDMSANKEIRDGPPVWNIGDEETKKIVELDLHANDYMDGQWGSMRHIVVKDEDVHVYKDCEHAFINTLTRGDVSSLTWFESPNQYFDSMVKSKATQELCSVYYAPINFRDIMLAYGRLPPDAIPGNFADRECLLGMEFSGRLKDGTRLMGILPAQALATTVMVDRDYAWEVPRDWTLAEASTVPVVYTTAYYALVRRGLMKKGDKILIHGGAGGVGQAAIAIALAAGCEVFTTVGSAEKREFLKNLFPQLQEHHFANSRSADFELHIRQHTKGRGVNIVLNSLANEMLQASLRCLARHGRFLEIGKVDLSQNSSLGMAKLLDNVSVHGILLDSIMDPTVGDLDEWKEIARLLEQGIKSGVVKPLHSHSFPADKAEEAFRFMSAGKHIGKVIMEIRPDEGTKVCPPSKISVRAICRTLCHPQHTYLITGGLGGFGLELAQWLINRGARKLVLTSRTGIRTGYQARCVHFWRRTGVSVLVSTLNIAKKSDAVELINQCTAMGPIGGIFHLAMVLRDCLFENQNVQNFKDAAEAKYYGTINLDYASREHCDKNILKWFVVFSSITSGRGNAGQTNYGWSNSCMERMIDQRRADGFPGIAIQWGAIGDVGVILENMGDNNTVVGGTLPQRMPSCLSSLDNFLSWNHPIVSSFIKAELGSKKNVGGGDLMATIAHILGVNDISQLNADANLSDLGLDSLMGVEIKQALERDHDIVLSMKEIRTLTLNKLQQLADQGGTGRTALQVNELEMKKDGERDAELNTAEMLEQQMNQLFKMRVDVNDLDPQDIIVKANKVEEGPITFFVHSIEGIATPLKKVMNKCEFPAYCFQSTKNVPQTSIEDVAKCYIREMKKIQPSGPYRLVGYSYGACIGFEMANMLQESDGRDAVERLILLDGSHLYMQTYRNVYRMAFGVTGDSLVNNPLFESEIMCAMTLRFANVDYKKFRFELLQQPGFKARVQKVVDQVMLTGLFKSPETVAFACEAMHSKFLMADKYKPRRNFGGHITLIRAEQGAAREEDVGEDYGVAAVSEDCEVLKVKGDHDTFVQGKSSSVTVEHINRIILQ

SEQ ID NO: 26 - DNA, 8936 bp, Rattus norvegicus
aggctgggctctatgggttgcctaagcggtctggaaagctgaaggatctgtccaagttcgacgcctccttttttggggtccaccccaagcaggcacacacaatggacccgcagctccggctgctgctggaagtcagctatgaagctattgtggacggaggtatcaacccggcctcactccgaggaacaaacactggtgtctgggtgggtgtgagtggttccgaggcgtcggaggccctgagcagagatcctgagactcttctgggctacagcatggtgggctgccagagagcaatgatggccaaccggctctctttcttcttcgacttcaaaggacccagcattgccctggacacagcctgctcctctagcctactggcactacagaatgcctatcaggctatccgcagtggggagtgccctgctgccattgtgggcgggatcaacctgctgctaaagcctaacacctctgtgcagttcatgaagctaggcatgctcagccccgatggcacctgcagatcctttgatgattcagggaacgggtattgccgtgctgaggctgtcgtggcagttctgctgactaagaagtccttggctcggcgagtctatgccactattctgaatgccgggacgaacacagatggctgcaaggagcaaggcgtgacattcccctctggagaagcccaggaacaactcatccgttctctgtatcagccgggcggtgtggcccccgagtctcttgaatatattgaagcccatggcacgggcaccaaggtgggggacccccaggaactgaacggcattactcggtccctgtgtgctttccgccagagccctttgttaattggctccaccaaatccaacatgggacaccctgagcctgcctcggggcttgcagccctgaccaaggtgctgttatccctagaaaatggggtttgggcccccaacctgcatttccacaaccccaaccctgaaatcccagcacttcttgatgggcggctgcaggtggtcgataggcccctgcctgttcgtggtggcatcgtgggcatcaactcgtttggcttcggaggtgccaatgttcacgtcatcctccagcccaacacacagcaggccccagcacctgccccacatgctgccctaccgcatttgctgcatgccagtggacggaccatggaggcagtgcagggcctgctggaacagggccgccagcacagtcaggacttggcctttgttagcatgctcaatgacattgcagcaacccctacagcagccatgcccttcagaggttacactgtgttaggtgttgagggccatgtccaggaagtgcagcaagtgcctgccagccagcgcccactctggttcatctgctcagggatgggcacacagtggcgtggaatggggctgagccttatgcgcctggacagtttccgtgagtccatcctgcgctctgatgaggctctgaagcccttgggagtcaaagtgtcagacctgctgctgagcactgatgagcacacctttgatgacatcgtgcattcctttgtgagcctcaccgccatccagattgccctcatcgacctgctgacgtctatggggctgaaacctgatggcatcattgggcactccttgggagaggttgcctgtggctatgcagatggctgtctctcccagagagaggctgtgcttgcagcctactggagaggccagtgcattaaggatgccaaccttccggctggatccatggcagctgttggtttgtcctgggaagaatgtaaacaacgctgccctcctggtgtggtgcctgcctgccacaactctgaggacactgtgaccatctctggacctcaggctgcagtgaatgaatttgtggagcagctaaagcaagagggcgtgtttgccaaggaggtgcgaacaggtggcctggccttccactcctacttcatggaaggaattgcccccacgctgctgcaggctctcaagaaggtgatccgggagccacggccacgctcagcacgctggctcagcacctctatccctgaggcccagtggcagagcagcctggcccgcacatcttctgctgagtacaacgtcaacaacctggtgagccctgtgctcttccaggaagcactgtggcacgtccccgagcacgccgtggtgctggagattgcaccccatgcactgttgcaggctgtcctgaagcgaggcgtgaagcctagctgcaccatcatccccttgatgaagagggaccataaagataacttggagttcttcctcaccaacctcggcaaggtgcacctcacaggcatcgacatcaaccctaatgccttgttcccacctgtggaattcccggttccccgagggactcctctcatctcccctcacatcaagtgggaccacagtcagacttgggatatcccagttgctgaagacttccccaacggttccagctcctcctcagctacagtctacaacattgacgccagttccgagtcacctgaccactacctggtcgaccactgcattgacggccgtgtcctcttccctggcactggctacctgtacctggtgtggaagacactggctcgaagcctgagcttgtccctagaagagacccctgtggtgtttgagaacgtgacatttcatcaggccaccatcctgcccaggacaggaaccgtgcctctggaggtgcggctgctagaggcctcacatgcatttgaggtgtctgacagtggcaacctgatagtgagcgggaaagtgtaccagtgggaagaccctgactccaagttattcgaccacccagaagtcccgatccccgccgagtccgagtctgtctcccgcttgacgcagggagaagtatacaaggagctgcggctacgtggctatgactatggccctcatttccagggcgtctatgaggccaccctcgaaggtgagcaaggcaagctgctctggaaagacaactgggtgaccttcatggacacaatgctgcagatatccatcctgggcttcagcaagcagagtctgcagctacccacccgtgtgactgccatctatattgaccctgcaacccacctgcagaaggtgtacatgctggagggagacactcaagtggctgacgtgaccacgagccgctgtctgggcgtgaccgtctctggtggtgtctacatttcgagactacagacaacagcaacctcacggcggcagcaggaacagctggtccccaccctggagaagtttgtcttcacaccccatgtggagcctgagtgcctgtctgagagtgctatcctgcagaaagagctgcagctgtgcaagggtctggcaaaggctctgcagaccaaggccacccagcaagggctgaagatgacagtgcctgggctagaggaccttccccagcatggactgcctcgactcttggctgctgcctgccagctgcagctcaacgggaacctgcaactggagttaggtgaggtactggctcgagagaggctcctgctgccagaagaccctctgatcagtggcctccttaactcccaggccctcaaggcctgcatagacacagccctggagaacttgtctactctcaagatgaaggtggtggaggtgctggctggagaaggccacttgtattcccacatctcagcactgctcaacacccagcctatgctgcaactggagtatacagccaccgaccggcacccccaggccctgaaggatgttcagaccaagctgcagcagcatgatgtagcacagggccagtgggacccttctggtcctgctcctaccaacctgggtgctcttgaccttgtggtgtgcaactgtgcgttagccaccctgggggatccagccctggccctggacaacatggtagctgccctcaaggatggtggtttcctgctaatgcacacagtgctcaaaggacatgcccttggggagaccctggcctgcctcccttctgaggtgcagcctgggcccagcttcttaagccaggaagagtgggagagcctgttctcaaggaaggcactgcacctggtgggccttaaaaagtcattctacggtactgcgctgttcctgtgccgccgtctcagcccacaggacaagcccatcttcctgcctgtggaggatactagtttccagtgggtggactctctgaagagcattctggccacatcctcctcccagcctgtgtggctaacagccatgaactgccccacctcaggtgtggtaggcttggtgaactgtctccgaaaagagccgggtggacaccggattcggtgtatcctgctgtccaacctcagcagcacatctcacgtccccaagctggaccctggctcttcagagctacagaaggtgctagagagtgatctggtgatgaacgtgtacagggacggtgcctggggtgccttccgtcacttccagttagagcaggacaagcccgaggagcagacagcacatgcctttgtaaacgtccttacccgaggggaccttgcctccatccgctgggtctcttctcccctgaaacacatgcagccgccctcgagctcaggagcacagctctgcactgtctactatgcctcactgaacttccgagatatcatgctggccacgggcaagctgtcccctgatgccattccaggtaaatgggccagccgggactgcatgcttggcatggagttctcaggccgtgataagtgcggccggcgtgtgatggggctggtacccgcagaaggcctggccacctcagtcctgttatcacccgacttcctctgggatgtaccctctagctggaccctggaggaggcggcttctgtgcctgttgtctacaccaccgcctactactccttagtagtgcgtggtcgtattcagcacggggaaactgtgctcattcactcgggctccggtggtgtgggccaagcggccatttccattgcccttagcctgggctgccgagtcttcaccactgtgggctccgctgagaagcgagcttacctccaggccagattccctcagctggatgacaccagctttgctaactctcgagacacatcgtttgagcagcatgtgttactgcacacaggtggcaaaggggtggacctggtcctcaactccctggcagaagagaagctgcaggccagtgtgcggtgcttggctcagcatggccgcttcctagagatcggcaaatttgatctttctaacaaccaccctctgggcatggccatcttcttgaagaacgtcactttccatgggatcctgctggatgcactttttgagggggccaacgacagctggcgggaggtggcagagctgctgaaggccggcatccgtgatggggttgtgaagcctctcaagtgtacagtgtttcccaaggcccaggtggaggacgccttccgatacatggctcaaggaaaacatattggcaaagtccttgtccaggtacgggaggaggagcccgaggctatgctgccaggggctcagcccaccctgatttccgccatctccaagaccttctgcccagagcataagagttacatcatcactggtggcctaggtggctttggcctggaactggcccggtggcttgtgcttcgtggggcccaaaggcttgtactaacttcccgatctggaatccgcacaggctaccaagccaagcacgttcgggagtggaggcgccagggcatccatgtgctagtgtcgacaagcaatgtcagttcactggagggggcccgtgctctcatcgctgaagccacaaagcttgggcccgttggaggtgtcttcaacctggccatggttttaagggatgccatgctggagaaccagactccagaactcttccaggatgtcaacaagcccaagtacaatggcaccctgaaccttgacagggcgacccgggaagcctgtcctgagctggactactttgtggccttctcctctgtaagctgcgggcgtggtaatgctggccaatccaactatggcttcgccaactctaccatggagcgtatttgcgaacagcgccggcacgatggcctcccaggtcttgccgtgcaatggggtgccattggtgacgtgggcattatcttggaagcgatgggtaccaatgacacagtcgttggcggcacactgccacagcgcatctcctcctgcatggaggtgctggacctcttcctgaatcagccccacgcagtcctgagcagttttgtgctggctgagaagaaagctgtggcccatggtgatggtgaagcccagagggatctggtgaaagcagtggcacacatcctaggcatccgcgacctcgcagggattaacctggacagctcgctggcagacctcggcctggactcgctcatgggtgtggaagtgcgccagatcctggaacgtgaacatgatctggtgctacccattcgtgaagtacggcaactcacactgcggaagcttcaggaaatgtcctccaaggctggctcagacactgagttggcagcccccaagtccaagaatgatacatccctgaagcaggcccagctgaatctgagtatcctgctggtgaaccctgagggccctaccttaacacgactcaactcagtgcagagctctgagcggcctctgttcctggtgcaccccattgaaggttccatcactgtgttccacagcctggctgccaagctcagtgtgcccacctacggtctgcagtgcacccaagcggcccccctggacagcattccaaacctggctgcctactacattgattgcatcaagcaggtgcagcctgaggggccctaccgagtggctgggtattcttttggagcttgtgtagccttcgagatgtgctcccagctgcaggcccagcagggcccagcccccgcccacaacaacctcttcttgtttgatggctcacacacctacgtattggcgtacacccagagctaccgggcaaagctgaccccaggctgtgaggctgaggctgaagctgaagccatatgcttcttcattaagcagtttgttgatgcagagcatagcaaggtgctagaggccctgctaccactgaagagcctggaggaccgggttgctgctgctgtggacctcatcactagaagccaccagagcctggaccgccgtgacctgagctttgctgccgtgtccttctactacaagcttcgagccgccgaccagtataaacccaaggccaagtaccacggcaatgtgatcctgctgcgggccaagacaggtggcacctacggcgaggacttgggtgccgattacaacctgtcccaggtgtgtgatgggaaggtgtctgtgcacatcattgagggtgaccaccgtacgctgctggagggcaggggcctggagtctatcatcaacatcatccacagctccctggctgagcctcgagtgagtgtacgggagggctagacctgcctaccatgaagccacgacccacaccggccaccagagatgctccgatccccaccacaccctgagtgcagggactggggagggtcctgctggtgggaccccctcaccccagtggcccagcaccaccccctcccctggtggctgctacaaacaggaccatcacatgtgtcccagccacttagtggggttcccagagccactgacttggaggcaccctggtctgtgaagagtcagtggaggccagcaagagccaaactgagccttttctgccaagtgacatttgtcacactggttgtttctccattaaattctcatatttattgcattgctgggaaagaccgcccaccccagggttaactcattccagaacccctaaagtgggaaaagccatgtggggaaggctgctggctggagcccctttttgtcttagccctgtacccgctcactgcagggcagggtatggagagggctggttcgcggggaacgaggaccccagcagacactgtagcccatggcccttggtccccagcactcccggctgcacccatgatgcagggcctaccagactctgcggaccgcaccgggcactcactgtatttgttttccaagattcaaattgctgcttgggttttgaatttactgcagctgtcagtgtaaagaaacatgtctgaactgtgtcctttttacaccaacctggtaaaaatgctcttgatgctgtcccgttgccacaattaaactgcacgtgagctctggcttccgttcagtctctttccagtcccagacctgagtccccagagcctccacagctcttacagtgagaatcaaattggcccactccttggaaggcgtggcattctgtcagagtaaaaggaaagtagagtgtgctgattcacgttcagcgtgtggggctggctagagaccttggcactgtagtgaacagaatgtgtccacctttaagtcaccctgaaggcatcaccatagctacagcctcacccaggggtagagaatagtactgtctacttgttgactacctggcagttggtgccagcccctatagaggaaaacagcagtgtgtggccactgtgagaagcatatccctggaaacaggtgaccagagcagagggctaacgcctacctgagtcacacaaaactgaccaggcttgagtgtccagaagagtctatcagaaggccacagcattcagtcctatccacagagagcagcagactaagttgtctccttgccagcttagaaaactgcagtgctggggtacaggtagggtgttcaggaggtccgggccccagtgattagtctaagactgaagcatctggttggctgtggtcccacctagaaaattcttaaagctcttgtcatgtacttcctgggaaggacctaccctgtctcaataatgtctctagctcgttggagtctactgactcaaacatttataaagtgtcctagaaaggcctgactcccctacaaggctgtgtgatccttcaaactcacatatgtgagccaataaaaccttgagactctag

SEQ ID NO: 27 - protein, 2431 aa, Rattus norvegicus
MDPQLRLLLEVSYEAIVDGGINPASLRGTNTGVWVGVSGSEASEALSRDPETLLGYSMVGCQRAMMANRLSFFFDFKGPSIALDTACSSSLLALQNAYQAIRSGECPAAIVGGINLLLKPNTSVQFMKLGMLSPDGTCRSFDDSGNGYCRAEAVVAVLLTKKSLARRVYATILNAGTNTDGCKEQGVTFPSGEAQEQLIRSLYQPGGVAPESLEYIEAHGTGTKVGDPQELNGITRSLCAFRQSPLLIGSTKSNMGHPEPASGLAALTKVLLSLENGVWAPNLHFHNPNPEIPALLDGRLQVVDRPLPVRGGIVGINSFGFGGANVHVILQPNTQQAPAPAPHAALPHLLHASGRTMEAVQGLLEQGRQHSQDLAFVSMLNDIAATPTAAMPFRGYTVLGVEGHVQEVQQVPASQRPLWFICSGMGTQWRGMGLSLMRLDSFRESILRSDEALKPLGVKVSDLLLSTDEHTFDDIVHSFVSLTAIQIALIDLLTSMGLKPDGIIGHSLGEVACGYADGCLSQREAVLAAYWRGQCIKDANLPAGSMAAVGLSWEECKQRCPPGVVPACHNSEDTVTISGPQAAVNEFVEQLKQEGVFAKEVRTGGLAFHSYFMEGIAPTLLQALKKVIREPRPRSARWLSTSIPEAQWQSSLARTSSAEYNVNNLVSPVLFQEALWHVPEHAVVLEIAPHALLQAVLKRGVKPSCTIIPLMKRDHKDNLEFFLTNLGKVHLTGIDINPNALFPPVEFPVPRGTPLISPHIKWDHSQTWDIPVAEDFPNGSSSSSATVYNIDASSESPDHYLVDHCIDGRVLFPGTGYLYLVWKTLARSLSLSLEETPVVFENVTFHQATILPRTGTVPLEVRLLEASHAFEVSDSGNLIVSGKVYQWEDPDSKLFDHPEVPIPAESESVSRLTQGEVYKELRLRGYDYGPHFQGVYEATLEGEQGKLLWKDNWVTFMDTMLQISILGFSKQSLQLPTRVTAIYIDPATHLQKVYMLEGDTQVADVTTSRCLGVTVSGGVYISRLQTTATSRRQQEQLVPTLEKFVFTPHVEPECLSESAILQKELQLCKGLAKALQTKATQQGLKMTVPGLEDLPQHGLPRLLAAACQLQLNGNLQLELGEVLARERLLLPEDPLISGLLNSQALKACIDTALENLSTLKMKVVEVLAGEGHLYSHISALLNTQPMLQLEYTATDRHPQALKDVQTKLQQHDVAQGQWDPSGPAPTNLGALDLVVCNCALATLGDPALALDNMVAALKDGGFLLMHTVLKGHALGETLACLPSEVQPGPSFLSQEEWESLFSRKALHLVGLKKSFYGTALFLCRRLSPQDKPIFLPVEDTSFQWVDSLKSILATSSSQPVWLTAMNCPTSGVVGLVNCLRKEPGGHRIRCILLSNLSSTSHVPKLDPGSSELQKVLESDLVMNVYRDGAWGAFRHFQLEQDKPEEQTAHAFVNVLTRGDLASIRWVSSPLKHMQPPSSSGAQLCTVYYASLNFRDIMLATGKLSPDAIPGKWASRDCMLGMEFSGRDKCGRRVMGLVPAEGLATSVLLSPDFLWDVPSSWTLEEAASVPVVYTTAYYSLVVRGRIQHGETVLIHSGSGGVGQAAISIALSLGCRVFTTVGSAEKRAYLQARFPQLDDTSFANSRDTSFEQHVLLHTGGKGVDLVLNSLAEEKLQASVRCLAQHGRFLEIGKFDLSNNHPLGMAIFLKNVTFHGILLDALFEGANDSWREVAELLKAGIRDGVVKPLKCTVFPKAQVEDAFRYMAQGKHIGKVLVQVREEEPEAMLPGAQPTLISAISKTFCPEHKSYIITGGLGGFGLELARWLVLRGAQRLVLTSRSGIRTGYQAKHVREWRRQGIHVLVSTSNVSSLEGARALIAEATKLGPVGGVFNLAMVLRDAMLENQTPELFQDVNKPKYNGTLNLDRATREACPELDYFVAFSSVSCGRGNAGQSNYGFANSTMERICEQRRHDGLPGLAVQWGAIGDVGIILEAMGTNDTVVGGTLPQRISSCMEVLDLFLNQPHAVLSSFVLAEKKAVAHGDGEAQRDLVKAVAHILGIRDLAGINLDSSLADLGLDSLMGVEVRQILEREHDLVLPIREVRQLTLRKLQEMSSKAGSDTELAAPKSKNDTSLKQAQLNLSILLVNPEGPTLTRLNSVQSSERPLFLVHPIEGSITVFHSLAAKLSVPTYGLQCTQAAPLDSIPNLAAYYIDCIKQVQPEGPYRVAGYSFGACVAFEMCSQLQAQQGPAPAHNNLFLFDGSHTYVLAYTQSYRAKLTPGCEAEAEAEAICFFIKQFVDAEHSKVLEALLPLKSLEDRVAAAVDLITRSHQSLDRRDLSFAAVSFYYKLRAADQYKPKAKYHGNVILLRAKTGGTYGEDLGADYNLSQVCDGKVSVHIIEGDHRTLLEGRGLESIINIIHSSLAEPRVSVREG

SEQ ID NO: 28 - DNA, 9345 bp, Gallus gallus
agaacctgctcaatggggttgatatggtcacagaggacgatcggaggtggaagccagggatttatggactgcccaaaagaaatggaaagctcaaggacataaaaaaattcgatgcctccttctttgggtccaccccaaacaagctcatacaatggatcctccagttcgcttgttgttggaagtttcttatgaggctattttggatggaggcattaatccaactgccctccgtggcacagacacgggtgtatgggttggtgcaagtggctcagaagctgctgaagcccttagccaagatccagaagagcttttgggatacagtatgactggctgccagcgtgctatgcttgccaacaggatttcttacttctatgattttacaggaccaagcttaactatcgacacagcctgctcctccagtctcatggctttagaaaatgcttataaagcaattcgtcacggacagtgcagtgcagccctggtaggaggggtcaacattctgctgaagcccaacacttctgtgcagttcatgaagctgggcatgcttagtcctgatggtgcctgcaaggctttcgatgtttcaggaaatgggtattgtcgctctgaagctgttgttgttgtgctcttgaccaagaaatccatggctaaacgcgtctatgccactatagtcaatgctgggagtaacactgatggctttaaggagcaaggtgtgacattcccatctggagagatgcagcagcagctggttggttctctgtacagagaatgtggtatcaagcctggagatgtggagtatgttgaagctcatgggacaggcaccaaggttggagatcctcaagaagtaaatggcattgtaaatgtcttctgccagtgtgagagagagcctctgttaattggatcaaccaagtcaaacatgggtcatccagagcctgcttctgggcttgctgcattagccaaggtcattctttctctggaacatggactgtgggctccaaatcttcatttcaatgatccaaatccagatattcctgctttacacgatggctccttgaaggtggtttgcaaaccaacaccggtgaaaggtggccttgtcagcatcaattcttttggctttggaggctctaatgctcatgttattctgaggccaaatgagaagaaatgtcagcctcaagagacttgtaacttgccaagactggttcaagtttgtggcagaacacaggaagctgtggaaatactaattgaagaaagcaggaaacatggaggatgcagtccatttttaagcctgctcagtgatatctctgcagttcctgtatcttctatgccctacaggggctacacactagttggcactgagagtgacataacagagattcagcaagttcaagcatctggtagaccactctggtacatctgctcaggcatgggaacacagtggaaaggtatgggcctgagccttatgaaattggatctgtttcgccagtctatattgcgctcagatgaggctttgaagagcacaggactgaaggtctcagacctgcttctgaatgcagatgagaacacttttgatgacactgtccatgcttttgttggactagctgctatacagattgcccaaattgatgtgctaaaggctgcgggtctgcaacctgatgggattttgggccactcagtgggagaactagcttgtggctatgcagataattccttaagtcatgaagaagctgttcttgctgcttattggaggggccgatgtgtgaaagaggccaaattgcccccgggagggatggctgctgttggtctgacatgggaggaatgtaagcagcgctgtcctccaaacgtggtaccagcatgtcacaactctgaggatactgtcactgtttcggggcctctggattctgtgtctgagtttgtaaccaaactgaagaaagatggggtgtttgcaaaggaggtgcgcagcgccggagttgcatttcattcctattacatggcatccattgcaccagcactgctcagtgcactgaaaaaggtcattccacaccctaagcctcgttcagcacggtggatcagtacatctatccctgaatctcagtggcagagtgatcttgctaggaattcctctgcagagtatcatgtgaacaacctagtgaatcctgtgctgttccatgaaggcctgaagcatattccagagaatgctgttgtagtggagattgctccacatgctctcttacaggctatcttgaggagaactttgaagccaacttgcactattctacctctgatgaagaaggaccacaaaaataacttggagttcttcctaacgcagactggaaagattcatttaactgggataaatgttcttggaaataacttgttcccacctgtggaataccctgtccctgtgggaacacctctcatttctccatatatcaaatgggaccacagccaagactgggatgttccaaaagctgaagacttcccctcaggttccaaaggctctgcgtctgcttcagtctacaacatcgatgtgagtcctgactctcctgaccattacttggttggccattgcattgatggcagagtcctgtacccagcaactgggtacttagtgctggcgtggcgaactctggcacgatctcttggcatggtcatggaacaaacagctgttatgtttgaagaagttacaatccatcaggcaactatccttcccaaaaagggatcaacacagctggaagtacgaatcatgcctgcttctcacagctttgaagtgtcagggaatgggaatttggctgtgagtgggaagatctccctcctagaaaacgatgctctgaagaactttcataaccagctggctgactttcagagtcaagcaaacgtgactgcgaagtctggcctcttgatggaagatgtttaccaagagctgcatcttcgtggatataactatggaccaacttttcagggtgttctggaatgcaacagtgaaggaagtgcagggaaaattctgtggaatggaaactgggtaaccttccttgacaccctgctacacttgatagtcttagcagagactgggcgcagtctacgattgcccaccaggattcgctcagtgtatattgaccctgtgcttcatcaggagcaggtgtaccagtaccaggacaatgtagaagcttttgatgttgttgttgaccgctgtcttgatagcctcaaagcaggaggtgttcagatcaatggacttcatgcctcggtggcaccacggcgacaacaggagcggatctctcccactctggaaaaattctcctttgttccctatattgagagtgactgtttgtcttccagtacccagcttcatgcctacctggagcactgcaaaggcctgatccagaaattacaagctaagatggcattgcacggagtcaaactagttatccatggcctagaaaccaacggggctgctgcaggatccccacccacacagaagggccttcagcatatccttactgaaatctgccatctggaactgaatggaaacctacattctgagctggaacagattgtgactcaggagaagatgcacctccaggacgatccccttctcaatggcttgctggattcttcagagttgaagacttgcctggatgtggcaaaggagaacacgaccagtcacaggatgaagatagtggaggctctggcaggaagtggacgtctgttctctcgtgtccaaagtattctgaatactcagcccctgttgcagctggactacattgccactgactgcacccctgaaactctttcaaatgatgaaacagagctgcacgatgctggaatctcctttagccagtgggatccctctagccttccctctggaaatctgaccaatgctgacctggcagtatgcaactgttcaacaagtgttctggggaacacagctgaaattatctctaacttagcagctgcagtgaaagaaggagggtttgttttgctgcacacccttcttaaagaggaaactcttggagaaattgtcagctttcttacaagtccagacctacagcaagagcacagcttcctgtctcaggcacagtgggaggagttattcagcaaggcctcattgaatctggttgcaatgaagagatctttctttggctcagttattttcctgtgtcgacggcagtcccctgccaaagcacccattcttctgccagtagatgacactcattataagtgggttgactccttaaaggagatcttggctgactcatcagagcagcctctgtggttgactgccaccaattgtgggaactctggaattttgggtatggtgaactgcctccgcctggaagcagagggccacagaatcaggtgtgtgtttgtttccaacctgagcccttcatcaactgtcccagccactagtctttcttccctggagatgcagaagattattgagagagatctggtgatgaatgtgtatcgtgatggaaagtggggttccttcaggcatctcccattgcagcaagctcagcctcaggagctgacagaatatgcctacgtaaatgtgttgactcgtggagatctctcttcccttcgttggattgtttccccacttcgacacttccaaacaaccaatccaaatgttcagctctgcaaagtctactatgcatctctcaatttccgggacattatgctggcaacaggaaagctttctccagatgctatccctggtaactggacgttgcagcagtgcatgctgggcatggagttctcaggacgggacctggctggaaggagagtgatgggattgctgccagcaaaagggctggcgacagtggtggactgtgacaagaggtttctatgggaagtgcctgaaaactggactctggaagaagcagcttcggtgcctgtggtttatgccactgcttattatgctttggtggttcgaggtggtatgaagaagggggagagtgtcctcattcactctggctcaggaggtgtgggcgcaagcagccattgccatcgccttgagcatgggctggcgcgtgtttttgctactgtaggctctgctgagaaacgtgagtatctccaagcaaggttcccacagctggatgctaatagctttgccagctcccgaaatacaacctttgagcaacacatactgcgagttaccaatgggaaaggtgtcaaccttgtgttaaattccttggcagaagagaagctccaagccagtttgcgttgtcttgctcaacatgggcgcttcttggaaataggcaaatttgatctatcaaacaacagccagcttggaatggctcttttcctcaagaatgtggcgtttcatggaatcctgctggattcaatctttgaggaaggaaaccaagagtgggaggtggtatcagagttgttgacaaaaggcataaaagatggtgtggtaaagcccctgagaaccacagtcttcggtaaagaagaggtagaagctgccttcaggttcatggcgcaaggaaaacatattggcaaagttatgatcaagatccaagaagaggagaagcaatatcctttaaggtctgaaccagtaaaactctctgccatctcccgaacttcctgcccacctaccaagtcttacatcatcacagggggcctaggaggatttgggcttgagttggcacagtggctaattgagagaggagcacagaagcttgtactgacatctcgatctggcatacgaactggctaccaggctaaatgtgttagagaatggaaggcgctgggaatccaagtgttggtctctaccagtgatgttggaactctagaaggaacgcagcttttgatagaagaggctttgaagctcggaccagttgggggcatctttaatttggctgtggtccttaaagatgccatgattgaaaatcagaccccggaattattctgggaggtcaacaagcccaagtattcaggcacccttcatttggactgggtgactcgtaagaagtgcccagacctggactattttgttgtattctcctctgtaagctgtggaagaggaaatgctgggcaaagtaattatggctttgctaattctgccatggagcgtatctgtgagcagcggcatcacgatgggctcccaggcctggcagtccagtggggagccattggtgatgtgggcatcctgaaggcaatgggaaacagggaggttgtgattgggggaaccgttctccagcaaatcagctcctgcctggaggtgctcgatatgttcctgaatcaacctcatcctgttatgtccagttttgtcctagcagagaaggtctctgtgaaaagtgaaggaggaagtcaacgggatcttgtagaagctgttgctcatatccttggtgttcgtgacgtgagcagtctgaatgctgagagctccctagcagacttgggcctggattccttgatgggtgtggaggtgcgccagacgctggagagagactacgacatcgtaatgaccatgagggagatccgactcctcaccatcaacaaactgcgtgaactgtcctccaagactgggacagcagaggagctgaagccatcacaagtgttgaagacaggcccaggtgagcctccaaaactggatttgaacaacttgctggtgaatccagaagggccaacgattacccgtctcaatgaagttcagagcacagaacgccctcttttccttgttcaccccattgagggatccattgcagtcttctatactcttgcctccaaacttcatatgccctgctatggactccagtgcacaaaagctgctcccttggacagcatacagagcctggcatcctattatattgactgtatgaagcagatacagcctgaaggaccttatcgcattgctggatactcttttggtgcctgcgtagcctttgaaatgtgctcccagctgcaagcacaacaaaatgcttcccatgcactcaacagtttattcctctttgatgggtctcattcctttgtggcagcatacactcagtgtttttccttttctctttttcagagctacagagcaaagctgacccaaggaaatgaggctgcgttggagacagaagcactgtgtgcctttgttcagcagtttacaggcattgaatacaataagttgttggagattcttctgcccttggaagatctggaggctcgtgtcaatgctgctgcagaccttataactcagattcataaaaacatcaaccgtgaagcactcagctttgctgctgcttccttttaccataagctgaaggctgctgacaagtatataccagaatccaagtatcatgggaacgtgacactgatgcgggcaaagactcacaatgagtatgaagaaggtctgggtggagactacagactctcagaggtctgcgatggaaaagtatcagtccacatcattgaaggagatcaccgcaccttattggagggagatggtgttgaatcaatcattgggatcatccatggctcactggcagagccacgtgtcagtgtcagagaaggttaacttctgccacttactgtcagtggtgaagaaaatgccaacaacattcctagttatgacagaccccaaggaactcttcctgttgaacaacatctcatctctctgctgccagagctgggaaggccagctgaacttgattggtctctttgtttcctctctcactcagtcatctttcctaactttcacgtgttctctctctctcctcttcccttcctatgctttgtctatttccccactatccctgcccgtgttactgcggtgctgtgactgtcactgtgcaccgggggttccccggcgatggtggcttcccacagctttggcagtatgtttttcaaatttaggagtagacttctacgtgctctatattgttttgtcttaacagtattccaaagggtaagtgatagcacttgttgaccaagcccagtgagcagagaggggaactgcagctgatttcggagatacctgttgtctgtgaagaatctgtctgtagtgaggtcagaaagagaattccatttgaggcttttgtaactatatttttttaatttgatatagtctaagtatttattgtgtcaaatcagagacttcttgctttgttttaatttatcgtgggtatcagaaaaggaaacatccgttttgaagggataggttcattctacaaggggaggttgcccatttgttaaaccaaagtgcatctatggaacagcccatttcttttttttttttaagttgattttttgtttgtgtttcgttttttgttgtttgttttttgtggcgttttgttaattttgattagtgatttttctgtgtgtggtttttctttcccccccccccccaccctgccttgttcagaagggtggaagtgaggttccttgccatcacccacccttgtggggagagaggcgtggagggcaggatggatggttcaacagatgccactgtattgaacagccttaacttgggctgatacaagcaggcagagctctccctaggtatgtacttagtttatatctctgcaaggttctgtgctttgcattaccagaaacacagtaaagcattacggctattgcttcacctttgttccttcccacctccagttgctccatccaaccaggcatttggaatgtcagggggaatagagttctccattggtcacggtataaatcctcctacccttgctctcccataaccaaagttcatgcaaacatagaaggcatctacccagtaccccagtgtattttatgtagcataggcttgcttaagccttgagtatgcattttcctctggcagtgagactggagatcccacataagttagctaagtaaaagtttgatggcatgattttaagatacagtacctttttaaaggaaacttgcataaaattcaatttaaaaatgactgacttttgctatgctggatctgtcttttccaaaatcagtaaatcctcttgacgcctatgatacagaggagacctgaatagcaatgaagtaccaaccaggaggcattccactgcctctcagaacttctgtaaacccctgttctttctgtattcatcccctagtgaagcatcctgtgagttcaggagcattccagtgagaggaacagctggttcctcgtggcaggttctacctagcgtctcttgcttatacaaccctctgtggagagtggctgggttaactggttttagttttataaagtatttcttttgtgaaatctgaaatacaaacaacataatgtcagcttaaagcatttctagaattaagttttgttttttacttttttttttttttttttttaatctgaagagtgtctttttcctctttggctttcctagaattaaacagaattgatcactg

SEQ ID NO: 29 - protein, 2447 aa, Gallus gallus
MDPPVRLLLEVSYEAILDGGINPTALRGTDTGVWVGASGSEAAEALSQDPEELLGYSMTGCQRAMLANRISYFYDFTGPSLTIDTACSSSLMALENAYKAIRHGQCSAALVGGVNILLKPNTSVQFMKLGMLSPDGACKAFDVSGNGYCRSEAVVVVLLTKKSMAKRVYATIVNAGSNTDGFKEQGVTFPSGEMQQQLVGSLYRECGIKPGDVEYVEAHGTGTKVGDPQEVNGIVNVFCQCEREPLLIGSTKSNMGHPEPASGLAALAKVILSLEHGLWAPNLHFNDPNPDIPALHDGSLKVVCKPTPVKGGLVSINSFGFGGSNAHVILRPNEKKCQPQETCNLPRLVQVCGRTQEAVEILIEESRKHGGCSPFLSLLSDISAVPVSSMPYRGYTLVGTESDITEIQQVQASGRPLWYICSGMGTQWKGMGLSLMKLDLFRQSILRSDEALKSTGLKVSDLLLNADENTFDDTVHAFVGLAAIQIAQIDVLKAAGLQPDGILGHSVGELACGYADNSLSHEEAVLAAYWRGRCVKEAKLPPGGMAAVGLTWEECKQRCPPNVVPACHNSEDTVTVSGPLDSVSEFVTKLKKDGVFAKEVRSAGVAFHSYYMASIAPALLSALKKVIPHPKPRSARWISTSIPESQWQSDLARNSSAEYHVNNLVNPVLFHEGLKHIPENAVVVEIAPHALLQAILRRTLKPTCTILPLMKKDHKNNLEFFLTQTGKIHLTGINVLGNNLFPPVEYPVPVGTPLISPYIKWDHSQDWDVPKAEDFPSGSKGSASASVYNIDVSPDSPDHYLVGHCIDGRVLYPATGYLVLAWRTLARSLGMVMEQTAVMFEEVTIHQATILPKKGSTQLEVRIMPASHSFEVSGNGNLAVSGKISLLENDALKNFHNQLADFQSQANVTAKSGLLMEDVYQELHLRGYNYGPTFQGVLECNSEGSAGKILWNGNWVTFLDTLLHLIVLAETGRSLRLPTRIRSVYIDPVLHQEQVYQYQDNVEAFDVVVDRCLDSLKAGGVQINGLHASVAPRRQQERISPTLEKFSFVPYIESDCLSSSTQLHAYLEHCKGLIQKLQAKMALHGVKLVIHGLETNGAAAGSPPTQKGLQHILTEICHLELNGNLHSELEQIVTQEKMHLQDDPLLNGLLDSSELKTCLDVAKENTTSHRMKIVEALAGSGRLFSRVQSILNTQPLLQLDYIATDCTPETLSNDETELHDAGISFSQWDPSSLPSGNLTNADLAVCNCSTSVLGNTAEIISNLAAAVKEGGFVLLHTLLKEETLGEIVSFLTSPDLQQEHSFLSQAQWEELFSKASLNLVAMKRSFFGSVIFLCRRQSPAKAPILLPVDDTHYKWVDSLKEILADSSEQPLWLTATNCGNSGILGMVNCLRLEAEGHRIRCVFVSNLSPSSTVPATSLSSLEMQKIIERDLVMNVYRDGKWGSFRHLPLQQAQPQELTEYAYVNVLTRGDLSSLRWIVSPLRHFQTTNPNVQLCKVYYASLNFRDIMLATGKLSPDAIPGNWTLQQCMLGMEFSGRDLAGRRVMGLLPAKGLATVVDCDKRFLWEVPENWTLEEAASVPVVYATAYYALVVRGGMKKGESVLIHSGSGGVGASSHCHRLEHGLARVFATVGSAEKREYLQARFPQLDANSFASSRNTTFEQHILRVTNGKGVNLVLNSLAEEKLQASLRCLAQHGRFLEIGKFDLSNNSQLGMALFLKNVAFHGILLDSIFEEGNQEWEVVSELLTKGIKDGVVKPLRTTVFGKEEVEAAFRFMAQGKHIGKVMIKIQEEEKQYPLRSEPVKLSAISRTSCPPTKSYIITGGLGGFGLELAQWLIERGAQKLVLTSRSGIRTGYQAKCVREWKALGIQVLVSTSDVGTLEGTQLLIEEALKLGPVGGIFNLAVVLKDAMIENQTPELFWEVNKPKYSGTLHLDWVTRKKCPDLDYFVVFSSVSCGRGNAGQSNYGFANSAMERICEQRHHDGLPGLAVQWGAIGDVGILKAMGNREVVIGGTVLQQISSCLEVLDMFLNQPHPVMSSFVLAEKVSVKSEGGSQRDLVEAVAHILGVRDVSSLNAESSLADLGLDSLMGVEVRQTLERDYDIVMTMREIRLLTINKLRELSSKTGTAEELKPSQVLKTGPGEPPKLDLNNLLVNPEGPTITRLNEVQSTERPLFLVHPIEGSIAVFYTLASKLHMPCYGLQCTKAAPLDSIQSLASYYIDCMKQIQPEGPYRIAGYSFGACVAFEMCSQLQAQQNASHALNSLFLFDGSHSFVAAYTQCFSFSLFQSYRAKLTQGNEAALETEALCAFVQQFTGIEYNKLLEILLPLEDLEARVNAAADLITQIHKNINREALSFAAASFYHKLKAADKYIPESKYHGNVTLMRAKTHNEYEEGLGGDYRLSEVCDGKVSVHIIEGDHRTLLEGDGVESIIGIIHGSLAEPRVSVREG

SEQ ID NO: 30 - DNA, 8391 bp, Mycobacterium bovis
atgggtacgcgcactggcggccgaggacccggttccgtccgacaagcacctgacgtcggccgccgtgtcggtgcccggcgtgttgcttacccagatcgcggcgacccgggcgctggcccgtcaaggcatggacctcgtggccaccccgccggtcgccatggcgggcattcgcaaggtgtgctggcggtggaagccctcaaggctggtggggcacgcgacgtcgagctgtttgccttggcccagttgatcggtgccgccggaacgctggtggcccgccggcgcgaatttccgtcctgggcgatcgcgccgatggtatcggtcaccaacgccgaccccgagcgcatcggccggttgctcgacgagttcgcccaggacgtgcgcacggtgctgccaccggtgttgtccatccgcaacggccggcgtgccgtcgtcatcaccggcacccccgagcagctgtcgcgtttcgagctttattgccgccagatctccgagaaggaagaagccgaccgcaagaacaaggtccgcggcggcgacgtcttctcgccggtcttcgagccggtgcaggtggaggtgggctttcacaccccgcggctatccgacgggatcgacatcgtcgcgggctgggccgagaaggcgggcctcgatgtcgccttggctcgggagctggccgatgccatcttgatcagaaaggtcgactgggtcgacgagatcacccgtgtccaccgggccggcgcccgctggatcctcgacctggggccgggcgacatcctgacccgactgaccgcaccggtgatccgcggcctgggcatcggcatcgtgccggcgcgtacccgcggtggccagcgcaacctgttcaccgtcggcgccacccccgaggttgcccgggcctggtcgagctacgcaccgaccgtggttcgcctccccgacggcagggtcaagctctcgacgaagttcacccggctgacccgccgctcgccgatcctgctcgcgggcatgaccccgaccaccgtggacgccaagatcgtcgccgcggcggccaacggccggcactgggccgagctggcggcgcggggcaggtcaccgaagagatcttcggtaaccgcatcgaacaaatggccggcctgctcgagccgggccgcacctatcagttcaacgcgctgttcctcgatccctacctgtgaagcttcaggtgggcggcaagcggttggtgcagaaggcccgccagtccggcgccgcgatcgacggcgtggtgatcagcgccggcatcccagacctcgacgaggccgtcgagctgatcgacgaactgggcgacatcggcatcagccacgtcgtgttcaaacccgggaccatcgagcagatccgctcggtgattcgcatcgccaccgaggtgcccaccaagccggtgatcatgcacgtcgagggccgggcgcgccggcgggcaccattcctgggaggatctcacacctgctgctggctacctactcggcagatcgggcaccgcgccaacatcacgtgtgcgtcggcggcggccatctcggcaccccgaagaagggctgcggatatttgtccgggcctgggcgcagcgtacggcttcccattgatgccgatcgacgcatcctggtcggcaccgcggcgatggccaccaaggaatccaccacgtcgccatcggtcaagcggatgctcgtcgacactcagggcaccgaccaatggatcagcgccggaaaagcgcagggccgcatgcctccagccgagtcagctcggtgccgacatccacgagatcgacacagcgcatccgtgcggcgctgctcgacgaggtggccggtgacgcggaggcggtcgcggagcgtcgcatggccaagaccgccaagccctacttgccgacgtcgccgacatgacctacctgcagtggctgcgggcgctacgtcgaactggccatcggggaaggcaactcgaccgccgacaccgcctcggtgggcagcccgtggctggccgacactggcgggaccgcttcgagcagatgctgcagcgtgccgaagcccggttgcacccacaggatttcggcccgatccagacgctattcaccgatgctggcctgctggacaatccgcagcagcgatcgccgccctcgtggcgcgctaccccgacgccgagaccgtgcagttgcatcccgcggatgtgccctttttcgtgacgttgtgcaagacgctgggcaagccggtcaacttcgtgccggcgatcgacctcgtcgtgcgcgctggtggcgcagcgactcgctgtggcaggcccacgacgcccgctacgacgccgatgcggtgtgcatcattccgggcacgcgtcggtagccgcatcacccggatggatgaacccgtcggtgagttgctggacgctttcgagcaagccgcaatcgatgaagtgctcggcgccggtgtcgagccgaaggatgtcgcgtccggccggctgggccgggccgacgtggccggaccgttggctgtcgtcctcgacgcacccgatgtgcgctgggccggtcgcaccgtgaccaacccggtgcatcggatccgcgacccggccgaatggcaggtgcacgatggacccgaaaacccgcgcgccgcacactcatccaccggcgcccggctgcagacgcacggcgacgacgtcgccttgagcgtcgcgcgtctcgggcacctgggtcgacatccgattcacgttgccggccaacaccgtcgatggcggcaccccggtgatcgccaccgaggacgccaccacgccatgcgcacggtgctgcgatcgccgccggtgtcgacagcccggagttcttgctgcggtggccaacgggacggccactttgacggtggactggcaccccgagcgtgttgccgaccacaccgtcaccgccacgttcggtgcgcgctggcacccagcctcaccaacgtgccgacgcgactcgtcggcccttgttggccagcggttttcgcggccatcggatcggcggtcaccgacaccggtgagccggtggtggaaggcctgctgagcctggtgcatctggacacgcggccgcgcgtggtcggtcagctgcccacggtcccggcccaattgaccgtcacgcaacggctgccaacgcaaccgatacggacatgggccgcgtcgtgccggtctcggtcgtcgttcaccgcatggcgccgtgatcgccactctcgaggagcgattcgcgatcctgggtcgcaccggttcgccgagctggaccggcgcgagccggtggcgcggtgtcgcgaacgccaccgacaccccgcgcgtcgccgccgcgacgtcacgatcaccgcgccggtcgacatgcgcccgttcgcggtggtgtccggcgaccacaaccccattcacaccgaccgggccgccgctgcttgccggcctggagtcgccgatcgtgcacggcatgtggctgtcggccgcggcgcaacacgcggtgaccggcaccgacgggcaggcccgccaccggcccggctggtcggctggaccgcgcggtttttgggcatggtggccccggcgacgaggtggacttccggtcgagcgcgtcggatcgaccagggcgcagagattgtggacgtggccgcgcgcgtcgggtcggatctagtgatgtcggcctccgcgcgactggccgcacccaagacggtctacgcattccccggccagggcatccaacacaagggcatgggcatggaggtgcgcgccgctccaaggcggcccgcaaggtgtgggacaccgcggacaagttcacccgcgacaccctgggcttctcggtactgcacgtggtccgcgacaacccgaccagcatcatcgccagcggtgtgcactaccaccaccgacggggtgctctacctgacgcagttcacccaggtcgcgatggcgacggtggcggccgggcaggtcgccgagatgcgtgaacagggagccttcgtcgaaggcgccatcgcgtgcggccactcggtcggcgagtacaccgcgctggcctgcgtgaccggcatctaccaactggaagccttgctggagatggtgtttcaccgcgggtcgaagatgcacgacatcgttccgcgcgacgagctcggccgctccaactatcggctgtcggccatccggccgtcccagatcgacctcgacgacgccgacgtgcccgcgttcgtcgccgggatcgcggagagcaccggtgaattcctggagatcgagaatttcaacctcggtggctcgcaatacgcgatcgcgggcacggtacgcggcctcgaggcgctcgaggccgaggtggagcggcgccgcgagctcaccggcggccgacggtcgttcattttggtgcccggcatcgatgttccgttccactcgcgagtgctgcgggtcggggtggccgaattccggcgctcgctggaccgggtcatgcggccgacgcggacccgacctgatcatcgggcgctacattcccaacctggtgccgcggaagttcaaccctggaccgcgacttcatccaggaaatccgggatttggtgccccgccgagccgctcgacgagatcctcgccgactacgacacctggcttcgcgacgaccggcgagatggcgcgcacggtgttcatcgagctgctggcatggcaattcgccagcccggtgcgctggatcgagacgcaggatctgctgttcatcgaggaggcgccggcgggctgggtgtggagcgattcgtcgagatcggtgtgaagagctcaccgacggtggcggggtcttgccaccaacaccctcaaactgcccgaatacgcccacagcacagtgaagtgctcaacgccgagcgtgatgcgcggtgctgttcgccaccgacaccgacccggagccggagccggaggaagacgagccggtcgcggaatcgcccgcgccggacgtcgtctcggaagccgcccccgtcgcgccggccgcttcgtcggcgggcccgcgtcccgacgatctggttttcgacgccgccgatgccacgctgcgtgatcgcgctctcggccaagatgcgcatcgaccagatcgaagaactcgactccatcgagtccatcaccgacggtgcgtcgtcgcggcgcaaccagctgctggtggacctgggctccgagctgaacctcggtgccattgaacggcgccgccgaatcggacctggccggtctgcgctcacaggtgaccaaactggcgcgcacctacaacgttacggcccagtgctttccgacgccatcaacgaccacgttcgcaccgtcctcggaccgtcgggcaagcggcccggcgccatcgccgagcgggtgaagaagacctgggagctcggtgaggctgggccaagcatgtcaccgtcgaggtcgcgctgggcacccgcgagggcagcagcgttcgcggcggcgccatgggccacctgcacgagggcgcgctggccgatgccgcctccgtcgacaaggtcatcgacgcggcggtcgcatcggtggccgcggccagggcgtttcggtagcgctgcgtcggccggtagtggcggcgccaccatcgacgcggccgcgctcagcgagttcaccgaccaaatcaccggccgtgagggcgtgctgcctccgcggcccgcctggtgctggggcagctgggactggacgaccccatcaaccgttgccggccgccccgattccgagctgatcgacttggtcaccgccgaactgggacggactggccgcggttggtggcaccggtgttcgaccccaagaaggccgtcgtattcgacgaccgctggccagcgcccgcgaggacctggtgaagctgtggctgaccgacggaaggaccgaaggcgacatcgacgccgactggccgcgctggcggagcgcttcgagggtgccgccacgtcgtggcgacccaggctacctggtggcaaggtaagtcgctcgcgcgggccggcagatccatgcatcgctgtacggccgcatgccgccggcgccgagaaccccgaaccccgcgtacggcggcgaagttgccgtggtgaccggcgcttcgaagggttcgatcgccgcgtcggtggtggctcggctgctcgacgcggagccaccgtcatcgcgaccacctccaagctcgacgaggagcggctgcggttctaccgcacgctgtatcgcgaccacgcccgttacggcgcggcgctgtggctggtcgcggcgaacatggcgtcctactccgacgtcgacgccctggtcgaatggatcggcaccgaacagaccgaaagccttgggccgcagtcgattcacatcaaagacgcgcagaccccgacgctgctgttccgttcgcggcgcacgcgtgtcgggactgtcggaggccggttcgcgcgccgagatggagatgaaagtgctgctgtggcggtgcaacggctgatcggcggcctgtcgacgatcggcgccgaacgcgacatgccgtcgcggctcgagcgtggtgctgcccggctcgcccaaccgtggcatgttcggcggcgacgggccctacggcgaagccaagtccgcgctggatgccgtggtgacgcgctggcacgccgagtcgtcctgggcggcacgggtcagcctggcgcacgcgctcatcggctggacccgcggcaccgggctgatgggccacaacgatgccatcgtggccgccgtcgaagaggccggggtcaccacctactcgaccgacgagatggcgcggctgctgctcgacctgtgtcatgcggaatccaaggtggctgcggccgttcgccgatcaaggccgacctgaccgggggcctgccgaggccaacctcgacatggccgagctggcggccaaggcgcgcgagcagatgtcggcagcggcggccgtcgacgaggacgccgaggcccctggcgccatcgccgcgctgccgtcgccgccccggtttcacccccgcaccgccgccgcaatgggacgacctcgatgtcgacccggccgacctggtggtgatcgtcggcggccgcgaaatcggcccgtacggctcgtcacgcacccggttcgagatggaggtcgaaaacgagctgtcggcggccggcgtgctggagctggcctggaccactgggttgatcgctgggagacgacccgcaacccggttggtacgacaccgaatccggcgaaatggtcgacgaatccgagttggtgcagcgctacacgacgccgtggtgcagcgcgtcggcattcgcgaattcgttgatgacggcgcgatcgaccccgaccacgcctcgccgctgctggtgtcggtgttcctggagaaggacttcgcgttcgtggtgtcctcggaggccgatgcgcgcgccttcgtcgagttcgatcccgagcacacggtcatccggccggtgcccgactccaccgactggcaggtcatccgcaaggccggcaccgagatccgggtgccgcgaaagaccaagctgtcccgcgtcgtcggcggccagatcccgaccgggttcgacccgacggtgtggggcatcagcgcagacatggccggttccatcgaccggttggcggtatggaacatgtggcggaccgtcgaccggttcctgtcgtccggtttcagcccggccgaggtgatgcgttacgtgcacccgagtttggtggccaacacccagggcaccggcatgggcggcggcacgtcgatgcagacgatgtaccacggcaatctgttgggccgcaacaagccgaacgacatcttccaggaagtcttgccgatatcattcgccgcgcacgtggttcagtcctacgtcggtagctacggtgcgatgatccacccggtagccgcgtgcgccaccgccgcggtgtcggtcgaggaaggtgtcgacaagatccggttgggaaggctcaactggtggtcggcggccgtggatgacctgacgctggagggcatcatcggattcggtgacatggccgccaccgccgacacgtccatgatgcgcggccgcggcatccacgactcgaagttttcccggcccaacgaccgccgccgtctggcttcgtcgaagcccaaggcggcgggacgatcctgttgggcgcggggacctggcgctgcggatggggctgccggtgctggcggtggtgggttcgcgcagtcgttcggcgacggcgtgcacacctcgatccgccccgggcctgggcgcgctgggggcggcgcgcggcggcaaggattcagctgcggcgggcgctggccaagctgcgtggccgccgacgacgtggcggtcatctccaagcacgacacctcgacgctggccaacgatcccaacgagaccgagttgcatgaacggctcgccgacgccctgggccgttccgagggcgccccgctgttcgtggtgtcgcagaagagcctgaccggccagccaagggcggcgcggcggtcttccagatga

SEQ ID NO: 31 - protein, 2796 aa, Mycobacterium bovis
MGTRTGGRGPGSVRQAPDVGRRVGARRVAYPDRGDPGAGPSRHGPRGHPAGRHGGHSQGVLAVEALKAGGARDVELFALAQLIGAAGTLVARRREFPSWAIAPMVSVTNADPERIGRLLDEFAQDVRTVLPPVLSIRNGRRAVVITGTPEQLSRFELYCRQISEKEEADRKNKVRGGDVFSPVFEPVQVEVGFHTPRLSDGIDIVAGWAEKAGLDVALARELADAILIRKVDWVDEITRVHRAGARWILDLGPGDILTRLTAPVIRGLGIGIVPARTRGGQRNLFTVGATPEVARAWSSYAPTVVRLPDGRVKLSTKFTRLTRRSPILLAGMTPTTVDAKIVAAAANGRHWAELAARGRSPKRSSVTASNKWPACSSRAAPISSTRCSSIPTCEASGGRQAVGAEGPPVRRRDRRRGDQRRHPRPRRGRRADRRTGRHRHQPRRVQTRDHRADPLGDSHRHRGAHQAGDHARRGPGAPAGTIPGRISHLLLATYSADRAPRQHHVCVGGGHLGTPKKGCGYLSGPGRSVRLPIDADRRILVGTAAMATKESTTSPSVKRMLVDTQGTDQWISAGKAQGRMPPAESARCRHPRDRHSASVRRCSTRWPVTRRRSRSVAWPRPPSPTCRRRRHDLPAVAAGATSNWPSGKATRPPTPPRWAARGWPTLAGPLRADAAACRSPVAPTGFRPDPDAIHRCWPAGQSAAAIAALVARYPDAETVQLHPADVPFFVTLCKTLGKPVNFVPAIDLVVRAGGAATRCGRPTTPATTPMRCASFRARVGSRITRMDEPVGELLDAFEQAAIDEVLGAGVEPKDVASGRLGRADVAGPLAVVLDAPDVRWAGRTVTNPVHRIRDPAEWQVHDGPENPRAAHSSTGARLQTHGDDVALSVARLGHLGRHPIHVAGQHRRWRHPGDRHRGRHHAMRTVLRSPPVSTARSSCCGGQRDGHFDGGLAPRACCRPHRHRHVRCALAPSLTNVPTRLVGPCWPAVFAAIGSAVTDTGEPVVEGLLSLVHLDTRPRVVGQLPTVPAQLTVTQRLPTQPIRTWAASCRSRSSFTAWRRDRHSRGAIRDPGSHRFAELDRREPVARCRERHRHPARRRRDVTITAPVDMRPFAVVSGDHNPIHTDRAAAACRPGVADRARHVAVGRGATRGDRHRRAGPPPARLVGWTARFLGMVAPATRWTSGRARRIDQGAEIVDVAARVGSDLVMSASARLAAPKTVYAFPGQGIQHKGMGMEVRAAPRRPARCGTPRTSSPATPWASRYCTWSATTRPASSPAVCTTTTDGVLYLTQFTQVAMATVAAGQVAEMREQGAFVEGAIACGHSVGEYTALACVTGIYQLEALLEMVFHRGSKMHDIVPRDELGRSNYRLSAIRPSQIDLDDADVPAFVAGIAESTGEFLEIENFNLGGSQYAIAGTVRGLEALEAEVERRRELTGGRRSFILVPGIDVPFHSRVLRVGVAEFRRSLDRVMRPTRTRPDHRALHSQPGAAEVQPWTATSSRKSGIWCPAEPLDEILADYDTWLRDDRRDGAHGVHRAAGMAIRQPGALDRDAGSAVHRGGAGGLGVERFVEIGVKSSPTVAGSCHQHPQTARIRPQHSEVLNAERDARCCSPPTPTRSRSRRKTSRSRNRPRRTSSRKPPPSRRPLRRRARVPTIWFSTPPMPRCVIALSAKMRIDQIEELDSIESITDGASSRRNQLLVDLGSELNLGAIERRRRIGPGRSALTGDQTGAHLQRYGPVLSDAINDHVRTVLGPSGKRPGAIAERVKKTWELGEAGPSMSPSRSRWAPARAAAFAAAPWATCTRARWPMPPPSTRSSTRRSHRWPRPGRFGSAASAGSGGATIDAAALSEFTDQITGREGVLPPRPAWCWGSWDWTTPSTVAGRPDSELIDLVTAELGRTGRGWWHRCSTPRRPSYSTTAGQRPRGPGEAVADRRKDRRRHRRRLAALAERFEGAATSWRPRLPGGKVSRSRGPADPCIAVRPHAAGAENPEPRVRRRSCRGDRRFEGFDRRVGGGSAARRGATVIATTSKLDEERLRFYRTLYRDHARYGAALWLVAANMASYSDVDALVEWIGTEQTESLGPQSIHIKDAQTPTLLFRSRRTRVGTVGGRFARRDGDESAAVAVQRLIGGLSTIGAERDMPSRLERGAARLAQPWHVRRRRALRRSQVRAGCRGDALARRVVLGGTGQPGARAHRLDPRHRADGPQRCHRGRRRRGRGHHLLDRRDGAAAARPVSCGIQGGCGRSPIKADLTGGLPRPTSTWPSWRPRRASRCRQRRPSTRTPRPLAPSPRCRRRPGFTPAPPPQWDDLDVDPADLVVIVGGREIGPYGSSRTRFEMEVENELSAAGVLELAWTTGLIAGRRPATRLVRHRIRRNGRRIRVGAALHDAVVQRVGIREFVDDGAIDPDHASPLLVSVFLEKDFAFVVSSEADARAFVEFDPEHTVIRPVPDSTDWQVIRKAGTEIRVPRKTKLSRVVGGQIPTGFDPTVWGISADMAGSIDRLAVWNMWRTVDRFLSSGFSPAEVMRYVHPSLVANTQGTGMGGGTSMQTMYHGNLLGRNKPNDIFQEVLPISFAAHVVQSYVGSYGAMIHPVAACATAAVSVEEGVDKIRLGRLNWWSAAVDDLTLEGIIGFGDMAATADTSMMRGRGIHDSKFSRPNDRRRLASSKPKAAGRSCWARGPGAADGAAGAGGGGFAQSFGDGVHTSIRPGPGRAGGGARRQGFSCGGRWPSCVAADDVAVISKHDTSTLANDPNETELHERLADALGRSEGAPLFVVSQKSLTGQPRAARRSSR

SEQ ID NO: 32 - DNA, 675 bp, Bacillus subtilis
atgaagatttacggaatttatatggaccgcccgctttcacaggaagaaaatgaacggttcatgactttcatatcacctgaaaaacgggagaaatgccggagattttatcataaagaagatgctcaccgcaccctgctgggagatgtgctcgttcgctcagtcataagcaggcagtatcagttggacaaatccgatatccgctttagcacgcaggaatacgggaagccgtgcatccctgatcttcccgacgctcatttcaacatttctcactccggccgctgggtcattggtgcgtttgattcacagccgatcggcatagatatcgaaaaaacgaaaccgatcagccttgagatcgccaagcgcttcttttcaaaaacagagtacagcgaccttttagcaaaagacaaggacgagcagacagactatttttatcatctatggtcaatgaaagaaagctttatcaaacaggaaggcaaaggcttatcgcttccgcttgattccttttcagtgcgcctgcatcaggacggacaagtatccattgagcttccggacagccattccccatgctatatcaaaacgtatgaggtcgatcccggctacaaaatggctgtatgcgccgcacaccctgatttccccgaggatatcacaatggtctcgtacgaagagcttttataa

SEQ ID NO: 33 - protein, 224 aa, Bacillus subtilis
MKIYGIYMDRPLSQEENERFMTFISPEKREKCRRFYHKEDAHRTLLGDVLVRSVISRQYQLDKSDIRFSTQEYGKPCIPDLPDAHFNISHSGRWVIGAFDSQPIGIDIEKTKPISLEIAKRFFSKTEYSDLLAKDKDEQTDYFYHLWSMKESFIKQEGKGLSLPLDSFSVRLHQDGQVSIELPDSHSPCYIKTYEVDPGYKMAVCAAHPDFPEDITMVSYEELL

SEQ ID NO: 34 - DNA, 714 bp, Brevibacillus brevis
atgatagaaatgttatttgtaaaggttccaaacgaaatcgataggcatgtgtttaacttcttgtcatcaaatgtgagtaaggaaaaacagcaggcgtttgttcgatacgttaatgtgaaagatgcttatcgttctcttttaggggaattgcttattagaaaatatttgatacaagtattaaacattcctaatgaaaacattctatttaggaaaaatgaatatggaaaaccttttgttgatttcgatattcattttaatatttcccactctgatgaatgggttgtatgtgcaatttcaaatcatcctgttggaattgatatcgagcgtatttcggagatagacattaaaatagcagaacaattttttcatgaaaatgaatatatatggttgcagtctaaagcccaaaatagtcaagtttcttctttttttgagctttggactattaaagaaagttatataaaagctattggtaaaggtatgtacataccgattaattcattttggattgataagaatcaaacacaaactgtaatttacaaacagaataaaaaagaacctgttactatttatgaaccagagttgtttgagggctacaagtgttcttgttgttctttgttttcttctgtaacgaacttgtctattactaaattgcaagtgcaagagttatgtaatttgtttctagattctacattttctgaaaataataacttttag

SEQ ID NO: 35 - protein, 237 aa, Brevibacillus brevis
MIEMLFVKVPNEIDRHVFNFLSSNVSKEKQQAFVRYVNVKDAYRSLLGELLIRKYLIQVLNIPNENILFRKNEYGKPFVDFDIHFNISHSDEWVVCAISNHPVGIDIERISEIDIKIAEQFFHENEYIWLQSKAQNSQVSSFFELWTIKESYIKAIGKGMYIPINSFWIDKNQTQTVIYKQNKKEPVTIYEPELFEGYKCSCCSLFSSVTNLSITKLQVQELCNLFLDSTFSENNNF

SEQ ID NO: 36 - DNA, 648 bp, Escherichia coli
ttgtcatcagtctcgaatatggtcgatatgaaaactacgcatacctccctcccctttgccggacatacgctgcattttgttgagttcgatccggcgaatttttgtgagcaggatttactctggctgccgcactacgcacaactgcaacacgctggacgtaaacgtaaaacagagcatttagccggacggatcgctgctgtttatgctttgcgggaatatggctataaatgtgtgcccgcaatcggcgagctacgccaacctgtctggcctgcggaggtatacggcagtattagccactgtgggactacggcattagccgtggtatctcgtcaaccgattggcattgatatagaagaaattttttctgtacaaaccgcaagagaattgacagacaacattattacaccagcggaacacgagcgactcgcagactgcggtttagccttttctctggcgctgacactggcattttccgccaaagagagcgcatttaaggcaagtgagatccaaactgatgcaggttttctggactatcagataattagctggaataaacagcaggtcatcattcatcgtgagaatgagatgtttgctgtgcactggcagataaaagaaaagatagtcataacgctgtgccaacacgattaa

SEQ ID NO: 37 - protein, 215 aa, Escherichia coli
MSSVSNMVDMKTTHTSLPFAGHTLHFVEFDPANFCEQDLLWLPHYAQLQHAGRKRKTEHLAGRIAAVYALREYGYKCVPAIGELRQPVWPAEVYGSISHCGTTALAVVSRQPIGIDIEEIFSVQTARELTDNIITPAEHERLADCGLAFSLALTLAFSAKESAFKASEIQTDAGFLDYQIISWNKQQVIIHRENEMFAVHWQIKEKIVITLCQHD

SEQ ID NO: 38 - DNA, 741 bp, Streptomyces verticillus
gtgatcgccgccctcctgccctcctgggccgtcaccgaacacgccttcaccgacgccccggacgacccggtgagcctcctcttccccgaggaggccgcccacgtcgcccgcgccgtccccaagcgcctgcacgagttcgccaccgtccgggtgtgcgcccgcgccgccctcggccggctgggcctcccgcccggtccgctgctgcccggccgacggggcgcgccgagctggccggacggggtggtggggagcatgacgcactgtcagggcttccggggcgccgcggtcgcccgggccgccgacgccgcgtcgctcgggatagacgccgagccgaacgggccgctcccggacggcgtcctcgccatggtctcgctgccgtccgagcgcgagtggctcgccggactggcggcccgccggccggacgtgcactgggaccggctgctgttcagcgccaaggagagcgtcttcaaggcgtggtacccgctgaccggcctggagctggacttcgacgaggccgagctggccgtcgatccggacgccgggacgttcacggcccggctgctggtgccgggaccggtggtcggcggccgtcggctggacgggttcgaggggcgctgggcggcgggcgagggcctcgtcgtcacggccatcgccgtcgcggcgccggccggtaccgcggaggaatcggcggaaggggccgggaaggaagcgactgcggacgaccggaccgccgtcccgtaa

SEQ ID NO: 39 - protein, 246 aa, Streptomyces verticillus
MIAALLPSWAVTEHAFTDAPDDPVSLLFPEEAAHVARAVPKRLHEFATVRVCARAALGRLGLPPGPLLPGRRGAPSWPDGVVGSMTHCQGFRGAAVARAADAASLGIDAEPNGPLPDGVLAMVSLPSEREWLAGLAARRPDVHWDRLLFSAKESVFKAWYPLTGLELDFDEAELAVDPDAGTFTARLLVPGPVVGGRRLDGFEGRWAAGEGLVVTAIAVAAPAGTAEESAEGAGKEATADDRTAVP

SEQ ID NO: 40 - DNA, 819 bp, Saccharomyces cerevisiae
atggttaaaacgactgaagtagtaagcgaagtttcaaaggtggcaggtgtaagaccatgggcaggtatattcgttgttgaaattcaagaggatatactcgcggatgagtttacgttcgaggcattaatgagaactttgccattggcgtctcaagccagaatcctcaataaaaaatcgtttcacgatagatgttcaaatctatgcagccagctgctgcagttgtttggctgctctatagtaacgggcttaaattttcaagagctgaaatttgacaagggcagcttcggtaagccattcttagacaacaatcgttttcttccatttagcatgaccatcggtgaacaatatgtagctatgttcctcgtaaaatgtgtaagtacagatgaataccaggatgtcggaattgatatcgcttctccgtgcaattatggcgggagggaagagttggagctatttaaagaagtttttagtgaaagagaatttaacggtttactgaaagcgtctgatccatgcacaatatttacttacttatggtccttgaaggagtcgtatacaaaatttactggaactggccttaacacagacttgtcactaatagattttggcgctatcagcttttttccggctgagggagcttctatgtgcataactctggatgaagttccattgattttccattctcaatggttcaataacgaaattgtcactatctgtatgccaaagtccatcagtgataaaatcaacacgaacagaccaaaattatataatatcagcttatctacgttgattgattatttcatcgaaaatgatggtttataa

SEQ ID NO: 41 - protein, 272 aa, Saccharomyces cerevisiae
MVKTTEVVSEVSKVAGVRPWAGIFVVEIQEDILADEFTFEALMRTLPLASQARILNKKSFHDRCSNLCSQLLQLFGCSIVTGLNFQELKFDKGSFGKPFLDNNRFLPFSMTIGEQYVAMFLVKCVSTDEYQDVGIDIASPCNYGGREELELFKEVFSEREFNGLLKASDPCTIFTYLWSLKESYTKFTGTGLNTDLSLIDFGAISFFPAEGASMCITLDEVPLIFHSQWFNNEIVTICMPKSISDKINTNRPKLYNISLSTLIDYFIENDGL

SEQ ID NO: 42 - DNA, 588 bp, Escherichia coli
atggtggaccaggcgcaggacaccctgcgcccgaataacagattgtcagatatgcaggcaacaatggaacaaacccaggcctttgaaaaccgtgtgcttgagcgtctgaatgctggcaaaaccgtgcgaagctttctgatcaccgccgtcgagctcctgaccgaggcggtaaatcttctggtgcttcaggtattccgcaaagacgattacgcggtgaagtatgctgtagaaccgttactcgacggcgatggtccgctgggcgatctttctgtgcgtttaaaactcatttacgggttgggcgtcattaaccgccaggaatacgaagatgcggaactgctgatggcattgcgtgaagagctaaatcacgacggcaacgagtacgcctttaccgacgacgaaatccttggaccctttggtgaactgcactgcgtggcggcgttaccaccgccgccacagtttgaaccagcagactccagtttgtatgcaatgcaaattcagcgctatcaacaggctgtgcgatcaacaatggtcctttcactgactgagctgatttccaaaatcagcttaaaaaaagcctttcaaaagtaa

SEQ ID NO: 43 - protein, 195 aa, Escherichia coli
MVDQAQDTLRPNNRLSDMQATMEQTQAFENRVLERLNAGKTVRSFLITAVELLTEAVNLLVLQVFRKDDYAVKYAVEPLLDGDGPLGDLSVRLKLIYGLGVINRQEYEDAELLMALREELNHDGNEYAFTDDEILGPFGELHCVAALPPPPQFEPADSSLYAMQIQRYQQAVRSTMVLSLTELISKISLKKAFQK

SEQ ID NO: 44 - protein, 16 aa, Artificial Sequence
  Synthetic peptide
SKHDTSTNANDPNESE

SEQ ID NO: 45 - protein, 14 aa, Artificial Sequence
  Synthetic peptide
QNKIRQDQINDSDT

SEQ ID NO: 46 - protein, 16 aa, Artificial Sequence
  Synthetic peptide
RINSDSYWDNLPEEQR

SEQ ID NO: 47 - protein, 14 aa, Artificial sequence
  Synthetic peptide
TLVERDENGNSNYG

SEQ ID NO: 48 - DNA, 21 bp, Artificial Sequence
  Synthetic primer
ttcataagatgtcacgccagg

SEQ ID NO: 49 - DNA, 20 bp, Artificial Sequence
  Synthetic primer
ggtacgcgtcatattccttg

SEQ ID NO: 50 - DNA, 22 bp, Artificial Sequence
  Synthetic primer
atgcctcaccgttgttcccgac

SEQ ID NO: 51 - DNA, 33 bp, Artificial Sequence
  Synthetic primer
ggccgaggcggcctaagcagtctcatacttctc

SEQ ID NO: 52 - DNA, 31 bp, Artificial Sequence
  Synthetic primer
ggccattacggccatgtacgctggcgctgag

SEQ ID NO: 53 - DNA, 18 bp, Artificial Sequence
  Synthetic primer
gccrttnadcatccangc

SEQ ID NO: 54 - DNA, 20 bp, Artificial Sequence
  Synthetic primer
gayganaargayrtnaargc

SEQ ID NO: 55 - DNA, 21 bp, Artificial Sequence
  Synthetic primer
gcgctacttccattgagtctg

SEQ ID NO: 56 - DNA, 20 bp, Artificial Sequence
  Synthetic primer
atgtgtccccacgcttctcc

SEQ ID NO: 57 - DNA, 34 bp, Artificial Sequence
  Synthetic primer
tggccgaggcggcttaaaccaactctgcaacagc

SEQ ID NO: 58 - DNA, 40 bp, Artificial Sequence
  Synthetic primer
ggccattacggccaacaatggcgcgtcccgagactgagca

SEQ ID NO: 59 - DNA, 6249 bp, Lipomyces starkeyi
atgtacgctggcgctgagactggcgtcgcaacgcctcacactcaggcctctctgcgtccgctctcgctccagcatggctcgctggagcacaccgtgtttgtaccaaccgcactctatctctcaatcctccatcttcgggacgaattcgctgccactcttcccactccaactgaagacttcgccggagacgaagagcctgcatccaactgcgagcttgttgcccgattcctcgcctttctagtcgctcaggtcgaggaagagccagaccagtatgatgacgttctcgcgctcgtccttgccgactttgaatctcgcttcctccgtgcaaatgaagtccatgccgtcgcagctgcgcttcctggggatctgtccaagcgtgaggtcgtcatcagcgcctactacgcagccaggctcgctgccaatcgtccaatcaaaggccacgattcggcgctgcttcgtgccgccgctgacggtcatgcttccattttcgcaatcttcggcggccagggaaatatcgaggaatactttgatgagctccgcaacgtctactccctctatcatggtcttattgacgactttgttcagcactgcgcgcgcgagttgctcaagcttgcggccgacgatcgcaccatgaaagtctactcgaatggcctcgacatcatgcgatggctccgcgagcccgagtctactcctgacctcgattatcttgtttcggcacctgtctcacttcctttgatcggtgtcactcagctcactcattacgctgccacttgcaagatcctcggcaaggagccaggtgaatttagatcacacttgtccggaacaactggtcactctcaaggtgtcgtcactgccgctgccatttcggcctcgactacatgggagtccttcttcgacgtctcgtccaagactctccagatcctgttctggatcggttgccgcgctcagcagacttatcctcgcacctctctcgcgccctccgtcctccaggactcggtcaacgaaggcgaaggcaaaccatcgcctatgctttcggtgcgcgaccttgtcaagtcccaggtccagaagcacgttgatttgacaaattcccatcttccaccagagaagcacgtctcgatctcgctcgagaacggtgcccgtaactttgtcgtcaccggtccgcctcagtcgctctacggtcttagcttgtcgctccgcaagtcccgtgcgccgcccggactcgagcagaacagagtcccatattcccagcgcaagctcaagttctccaatagattcttgcccatcactgctcctttccactcgccgtacgttcatgaggcatacgaaaccatcgttgatgatctcaaatctgccaatgtttcgtttgcgccggaggagctcgccatcccagtctacgatacatatgatggacatgacttgcgcgagcttaccgacgaggacggctccgttgtcgagcgtcttgtcaagatggttaccagcctgcctgtcaactgggagcaagccacagccttcggcaaggtcacgcacattctcgactttgggcctggcggtgtgtctggcctaggtgtcttgacccatcgcaataaggagggaactggtgtccgtgtcatcttagccggcactttggagggtacggtatcggagcttggttacaagtccgaattgtttgatcgcgaggacgacgctgtcaaatttgccgctgactggggtcttgagtttgcacccaaacttgtcaagactgcgcaagggcagacctatgttgacacgaagttttcgcgtttacttggtcagccgcccatcatggttgccggtatgactccgtcaaccgttccgcctgatttcgttgctgccactatgaacgccggataccacatcgaacttggcggtggtggctactttaacgccagtggtctcacccaagcgttctacaagatcgaaaaatctactttccctggcgccggtattacagtcaatttgatctatgtcaatccccgcgccatgggatgggcaatccctctcattcagaagctccgcgcagaaggcgttccgatcgagggtctcaccatcggcgcaggtgttccatcaaccgaagtcgcgaatgagtacatcgaaactctcggcatcaagcacctgggtctcaaacctgggtctatagacgcgatccagcaggtcattaccatcgcgcaggccaatccaacattcccgatcgtcttgcagtggacaggaggtcgtggtggcggtcatcattcgttcgaggacttccacagcccgatcttgcagatgtacccgcgcatccgtcggtgcagcaacatcatcctcctcgcgggctcaggtttcggtggtgccgaagacacgtacccgtacttgactggtcagtgggcgacaagattcgcgtacccgccaatgccatttgacggtgtcttatttggcagcagaatcatgactgcgaaggaagcgcacacgtcactgggagcaaagcaagctattgttgatgcgcctggtgtggatgagttgcagtgggagaaaacatataatggtgctgctggtggcgtcattacagtcttgtccgagatgggcgagcccattcacaagttggctacgcgaggtgttgtgttttggaaggagatggatactacgattttcagtctgcccaagaacaagcgcgtcgatgccctcaaggccaagaaggattacatcatcaagaagcttaatgccgacttccaaaaggtttggttcggtaagaactctgccggtgaggtagtcgaactcgaggacatgacctacggtgagattctcaagcgcctagttgaactcatgtttattgcgcacgagaagcgctggatcgacctttcgctgcgcaacatgaccggtgactatatccgccgtatcgaggagcgcttcacgcatgagacaggtcgcccatcgctcctgcagtcgtacaccgagctggatgagccgacgccgactgttgacagaatcctggcggcgtatcccgaggccactgagcagattatcaacgtgcaagacaaggagtttttccttatgctgaccctccgacctggtcagaagccggttccgtttgttccggcgttggatgataattttgagttgtacttcaagaaggactcgctctggcagtctgaggaccttgctgccgtcgtcggtcaggatgtgcagcgcacatgtgttctgcagggtcctgtcgctgtcaagtacgcccagatcgtcaacgaacccgtgaaggacattttggatggcattcacgacaaacatatcgagctcttgaccaaggacatttatggaggagaggagtcgaagattccggttatcgaatactttggaggcaaggatattgttccatctgtctttgagaccgcattgaaggtagacagtttgaccgttaccgagtccgacgactccatcacctatgtgctcgatgccggtgtcagcggcaacgctactcttcccgatgttgagtcgtggttgagtttgcttggtggtgaacgttatggctggcgccacgcattcttcaccaccgatgtttttgttcagggcacgaagtatgagacgagcccgcttaagcggttgttcaagccggcgtttggggtcaaggtcaccattcagcatccggaagatttggagaagactcgcattatcgtgtccgagaaaatcaacggcaaggatgtcgtcgttattgacaccttcaagcatccggattctaataccatcgaaatgacgatttatgatgatcgtactgcagagggcaagcctgttggcatgctgctgttgttcacgtaccaccctgaagtcggatttgcgccaatccgcgaggtcatggccggccgcaacgacagaatcaaggagttttactggaagctgtggttcggtcctgaggagtacccggccaacttatccgtcaccgacaccttcgaaggtggctcgaccaccgtcactggcaaggcaattgctgactttgtatatgctgtcgggaacaacggtgaggcatttgtcgaccggcctggtaagtctacattcgcacctatggattttgctattgtagtagggtggaaggctattacgaaggcgctcttccccaaggccatcgacggtgacttgttgaagctggtccatctttcgaactcgttcaaaatgtaccctggtgcagagccgctcaagaaggacgacgttgttaccaccactgccaagatcaatgctgtgttgaaccaggagtccggtaagatggttgaagtcagcggcgttatctcccgtgattacatgccagtcatggaggtcacctcccagttcttttacagaggcgcgtacgccgactacgagaacacgttccagcgcaagtcggaattgccgatggaggtcacgctcaagtctcccaaggatgttgcggttttgcgatcgaaggactggttcgagctcaatgacgatcctcatgtcgacctactcaaccagactctcacgttccggctcgaaaccttcgtcagataccaaaacaagactgtcttttcgtctgtacgcaccactggtcaggttttgcttgaattgccgacgcgtgagattatccagatcggcaccgttgagtacgaggccagtgaatcccatggcaacccggtcatcgactatcttgagcgccacggcagcacgatcgaacagcctattatgttcgaaaactcgatcccattgaacgcctcgacggagcttgtctaccgcgcgcctgcttccaacgaaggctacgctcgtgtgtctggtgactacaacccgatccatgtgtcccgtgtgttcgcggagtatgccaacctccgaggcaacatcactcacggcatgtactcctccgccgccgtccggtcgctcgtcgagacctgggcagcggagaaccatgtcgcacgcgtgcgcgggtttaattgctctttcgtcggtatggtcctgccaaacgagaatatcgagacgaaattgcaccatgtcggtatgattgcgggccggaagattatcaaggttgagaccacgaacaaggactcaggtgatgtcgtcttgatcggccaggcagaggtcgagcagcccgtgtcgacgtatatcttcactggccagggttcgcaggagcagggtatgggaatggatctatacgagtctagcgctgtagcccgtgaggtttgggaccgcgctgatagacatttcctcaataactatggcttttcaattatcaatattgttaagaacaatcctaaagagtttactgttcactttggtggtccaagaggcaaggcaattcggcacaactacacatcgatgatgttcgagtctgttgatgcggacggtcagctcaagtccgagaagatcttcaaggacatcacggagaacacgtcgtcttataccttccgttcgcctactggcttgttgtcagccactcagttcacgcagccggctttgacgcttatggagaaggcttcgttcgaggacatgaacgccaagggtcttgtcccggctgactgcacgtacgcaggccactcattgggtgaatactctgcgctcgcggcactcggcgacgtcatgccgatcgagtctcttgtagatgtcgtgttctaccgtggtatgaccatgcaggtcgcagtcccgcgtgacgcgctaggacgatcgaattatggtatgtgcgctgtcaacccgtctcgtatctcgccgaccttcaacgatgccgcactccgctatgtcgtcgaacacatatcctctcagaccaagtggctcttggagattgtcaattacaatgtggagaacacgcaatacgtcactgccggagatctccgtggacttgactgtcttacgaacgtgctcaatttcatgaaggtgcagaagatcgacctcgacaagctcatgaagaccatgtcgatggaagatgttaaggagcagctcaccgacatcgtcgaagaaatagcgaagaagagcatcgcaaagccgcagccgatcgagctcgaccgtggtttcgccactatccctctcaaaggcatttcggtgccgttccactccagttatctgcgcagtggtgtcaagccgtttaaccggttcttgatcaagaagttgccacagcaggcgctcaagccggccaatttaattggaaagtatattccaaatttgactgccaagccattctcgatctcgaaggagtatttccaggaagtgtatgacttgaccggtagcgccaagatcaggagcattttggacaactgggagaagtatgagactgcttag

SEQ ID NO: 60 - protein, 2082 aa, Lipomyces starkeyi
MYAGAETGVATPHTQASLRPLSLQHGSLEHTVFVPTALYLSILHLRDEFAATLPTPTEDFAGDEEPASNCELVARFLAFLVAQVEEEPDQYDDVLALVLADFESRFLRANEVHAVAAALPGDLSKREVVISAYYAARLAANRPIKGHDSALLRAAADGHASIFAIFGGQGNIEEYFDELRNVYSLYHGLIDDFVQHCARELLKLAADDRTMKVYSNGLDIMRWLREPESTPDLDYLVSAPVSLPLIGVTQLTHYAATCKILGKEPGEFRSHLSGTTGHSQGVVTAAAISASTTWESFFDVSSKTLQILFWIGCRAQQTYPRTSLAPSVLQDSVNEGEGKPSPMLSVRDLVKSQVQKHVDLTNSHLPPEKHVSISLENGARNFVVTGPPQSLYGLSLSLRKSRAPPGLEQNRVPYSQRKLKFSNRFLPITAPFHSPYVHEAYETIVDDLKSANVSFAPEELAIPVYDTYDGHDLRELTDEDGSVVERLVKMVTSLPVNWEQATAFGKVTHILDFGPGGVSGLGVLTHRNKEGTGVRVILAGTLEGTVSELGYKSELFDREDDAVKFAADWGLEFAPKLVKTAQGQTYVDTKFSRLLGQPPIMVAGMTPSTVPPDFVAATMNAGYHIELGGGGYFNASGLTQAFYKIEKSTFPGAGITVNLIYVNPRAMGWAIPLIQKLRAEGVPIEGLTIGAGVPSTEVANEYIETLGIKHLGLKPGSIDAIQQVITIAQANPTFPIVLQWTGGRGGGHHSFEDFHSPILQMYPRIRRCSNIILLAGSGFGGAEDTYPYLTGQWATRFAYPPMPFDGVLFGSRIMTAKEAHTSLGAKQAIVDAPGVDELQWEKTYNGAAGGVITVLSEMGEPIHKLATRGVVFWKEMDTTIFSLPKNKRVDALKAKKDYIIKKLNADFQKVWFGKNSAGEVVELEDMTYGEILKRLVELMFIAHEKRWIDLSLRNMTGDYIRRIEERFTHETGRPSLLQSYTELDEPTPTVDRILAAYPEATEQIINVQDKEFFLMLTLRPGQKPVPFVPALDDNFELYFKKDSLWQSEDLAAVVGQDVQRTCVLQGPVAVKYAQIVNEPVKDILDGIHDKHIELLTKDIYGGEESKIPVIEYFGGKDIVPSVFETALKVDSLTVTESDDSITYVLDAGVSGNATLPDVESWLSLLGGERYGWRHAFFTTDVFVQGTKYETSPLKRLFKPAFGVKVTIQHPEDLEKTRIIVSEKINGKDVVVIDTFKHPDSNTIEMTIYDDRTAEGKPVGMLLLFTYHPEVGFAPIREVMAGRNDRIKEFYWKLWFGPEEYPANLSVTDTFEGGSTTVTGKAIADFVYAVGNNGEAFVDRPGKSTFAPMDFAIVVGWKAITKALFPKAIDGDLLKLVHLSNSFKMYPGAEPLKKDDVVTTTAKINAVLNQESGKMVEVSGVISRDYMPVMEVTSQFFYRGAYADYENTFQRKSELPMEVTLKSPKDVAVLRSKDWFELNDDPHVDLLNQTLTFRLETFVRYQNKTVFSSVRTTGQVLLELPTREIIQIGTVEYEASESHGNPVIDYLERHGSTIEQPIMFENSIPLNASTELVYRAPASNEGYARVSGDYNPIHVSRVFAEYANLRGNITHGMYSSAAVRSLVETWAAENHVARVRGFNCSFVGMVLPNENIETKLHHVGMIAGRKIIKVETTNKDSGDVVLIGQAEVEQPVSTYIFTGQGSQEQGMGMDLYESSAVAREVWDRADRHFLNNYGFSIINIVKNNPKEFTVHFGGPRGKAIRHNYTSMMFESVDADGQLKSEKIFKDITENTSSYTFRSPTGLLSATQFTQPALTLMEKASFEDMNAKGLVPADCTYAGHSLGEYSALAALGDVMPIESLVDVVFYRGMTMQVAVPRDALGRSNYGMCAVNPSRISPTFNDAALRYVVEHISSQTKWLLEIVNYNVENTQYVTAGDLRGLDCLTNVLNFMKVQKIDLDKLMKTMSMEDVKEQLTDIVEEIAKKSIAKPQPIELDRGFATIPLKGISVPFHSSYLRSGVKPFNRFLIKKLPQQALKPANLIGKYIPNLTAKPFSISKEYFQEVYDLTGSAKIRSILDNWEKYETA

SEQ ID NO: 61 - DNA, 5565 bp, Lipomyces starkeyi
atggcgcgtcccgagactgagcaagagctcgcccatatcctactcgtcgagctactcgcctaccagttcgcgtcccccgtccgctggatcgaaactcaggatgtcatcttccaggagttcaactccgaacggctcgtcgaaatcggtccctccccgacccttgcgggaatggcacagcgcactctaaaggcaaaatacgagtcatacgatgctgcactctcacttcagcgccaggttctctgctactctaaggacgccaaagagatctactacaccccagatcccgtcgtcgtcgaagccgctccagagcctgctgcggctactgctggcgctcctgctgctgctgccgttgctgccgttgctgctgctcctgctccatccggaccagttgccgctgtttctgatgagcctgtcaaagctgtcgaaatcttgcgctcgctcgtcgctcagaaactcaagaagccttacgaccaggtgccacttgcaaaggccattaaagatctcgtcggcggcaagtccactctccagaacgaaattctcggtgatctgggcaaggagttcggctcggctcctgagaaggcagaggagactcctctagaagagctaggcgccgctatccagggttccggcttcaatggccagctcggcaaacagtcactctcgctcatcggtcgactagtcgcttctaagatgcctggtggcttcaacctcacctccactcgcaagtatctgcaggatagatggggtcttggacctggtcgccaggacggtgttttgctcctcgccatcacgatcgagccacctgcacgtctcgcggccgaagccgatgcaaagaagtatctcgacgaagtcgctgcgaagtacgcttccttcgccggcatctcgctctcgtccggcggtggtgacgccggtgctggtggtgcgggaggcggcgctattgccatcgactctgccgcgttcgaggagctcaccaaggaccaaacccatctcgttcgccagcagatggaattgtttgccaagtacctcaaggtcgacctccgggccggcgataagcttttcgtcgacgaggtggacgcgtcctccgaactccgcaaggaacttgacctctggattgccgaacacggcgacttttacgcgaccggcatcgttccctccttctcgccgctcaaggcccgcgtgtacgactcgtcttggaattgggctcgccaggatgcgcttaccatgtactacgacatcatctttggccgtctgtctgttgtcgatagggagattgtgtcccactgcattctcctgatgaaccgctcgaaccctactttactcgaattcatgcagtatcatatcgaccactgcccggagaagcgtggggacacataccacctcgcgaagcagcttggccagcagctcatcgacaactgccgagatgcactgggcgtggagccagtctataaggacgtcatgtacccaacagctccccagaccgccattgatgttaaaggaaacatcaagtacgacgaagtccctcgtgtcgcagtccgcaagctcgagcaatacgtcaaggaaatggccgcaggtggcaagatcacggagaatcgcagccgcacgaagttgcatgccggccttgcccgcatctacaagataatccgccagcaacaaaaacttagcaagagttccaagcttcagatcaagaccttgtacgaggacgtcatccaatctctctctcttgaatccggtcttgccaacggcagtccctcacccgatggcacaggccgcccaacttcgcccaagcgcaggaagaacggcaagggaaagaaatacaccgagacgattccgttcttgcacttgaagaagaaggatatgcacggttgggactacagcaagcctctgactggcatctatctcgagtgcctcgagcaggctgcaaaatccggtatctcatttaaagacaagtgcgctttgatgaccggtgctggcgctggctctattggtgccgctgtcttgcagggacttttgtccggtggtgccaaggttgttgtcactacgtcgcgatattcgaaggaggtgacagagtactatcaatctatctacgccaagtacggcgccagcaacagtacattgattgtcgttcccttcaatcagggctctaagcaggacgttgacgctctcgttgactacgtttacgacaccaagaagggtctcggctgggatcttgattatgtcattccgttcgccgccattccagagaacggccgtgagatcgacggcattgactccaagtccgagcttgctcaccgaatgatgcttactaacttactccgcattctcggcaatgttaagactcagaagctcgctcacggctacgcgactcgtccggctcaggtcatcctaccgatgtcccccaaccacggtactttcgggtccgacggcttgtattcggagtctaagcttgcactcgagacattgttcaacagatggtactccgagtcctggggtccgtacctcacgatctgcggtgccgtcattggctggactcgtggcaccggtcttatgagccagaacaacttgatcgctgaacgtattgagggtcttggtgtccgcactttctctcagcaggagatggctttcaacatcctcggtcttatgtcccccgcgatcgtcaacctgtgccagatcgagcctgttttcgcggacttgaatggtggcatgcagtatatccccaatctcaaggaggcctcagcccagattcgtcaggagctcctccagacgtccgagattcgccgggcagtttccgcagagagtgcgatcgagtacaagcttgtcaatggcgcagaagctgagcgtctgcaaaagtctgtcgtcatccagccgcgcgccaatattaaattcgagttcccgaggctcaaggagtactcagaaatcgcccaccttgccgaggacctcaagggcatggtcgacctcgagaaagttgtcgtcgtcactggtttcgccgaagtcggtccttggggtaacgctcgcacgcggtgggagatggaagcctatggccagttctctcttgaaggttgcatcgaaatggcttggattatgggtctcattaagcaccacaatggtcaattgaagggcaagatgtactctggctgggtcgataccaagagcaacgagcctgtggacgactttgacgtcaagtcgaagtacgagaaacatattcttgagcactccggcatccggttgattgaggctgagttgtttgacggctatgaccccaagaagaagaagatgcttcaggaagttgttatcgagcatgatctcgagccattcgagacctcgaaggagaccgcgtatgagttcaagcgcgagcacggcgacaaggtcgagattttcgagattgctgagacgggacagtggactgtccgactgctcaagggtgccagtctgctaattcccaaggcgctccagttcgatcgtttggttgctggtcagatcccgaccggctgggacgcgcgacgatacggtatcggggaggacatcatttcgcaggtcgatccgatcactctctatgtgcttgtctccactgtcgaggcactgctttctgccggtgttaccgacccgtacgagttctacaagtacgtccatgtttctgaggtcggtaactgtaccggttccggtgtcggtggtatgagcgctcttcgcggcatgtacaaggaccgatttatggataagccggtccagaaagatattcttcaggaatctttcatcaacactatgtctgcatgggttaacatgttgctcctctcctcgtccggtccaatcaagactcctgtcggtgcctgcgctacttccattgagtctgtcgatattggctacgagaccatcatttccggcaaggccaagatctgtctggtcggcggctacgacgatttccaggaagaaggttcattcgagttcggcaacatgggtgccacatccaactccgagaccgagattgcccatgggcgtaccccagctgagatgtcccgtccgacgacgaccacgcgtgccggtttcatggagtcccatggtgctggtatccagttgattatgacggccaagctcgctctcgcaatgggtgtcccggtctacggtataattggtatgaccgccaccgcgaccgacaagatcggccggtccgtgccggcgcctggtaagggtatcctcaccacggcgcgtgagaacagggagtccaagttcccgtcgccactgcttaacatcaagtaccgacgccgccagcttgagaagcgtcagtcccagatccgcgagtggaccgagtccgagatcttgtatttgcagcaggaagtcatttccatgcgcgaacaatatgagaatttcgatgagagggagtatttgaccgagcgcctggcccatgttgagcgtgaggctgcgcgacaggagaaggacgccttggcgcagtggggtaatgatttctacaagcaagatgcaagaatcgcgcctctgcgtggtgcacttgctgtttggaatcttacggttgatgatcttggtgttgcgtcgttccacggcacgtccactgttgcaaatgataagaacgagagcatgacaattgccaacatgatgactcatctcggacgatcaaagggtaatgccatccttggtattttccagaagtatttgaccggtcatcctaagggtgccgcaggtgcttggatgttgaacggtgcgctccaggtcctcaacactggtctggtgcctggtaaccgcaatgccgacaacgttgacaaggtcctcgaacagtacgactcgattttgtatccgtcacgcagtatccagacggacggcatcaaggccgcgtccgtgacgtcgttcggtttcggccagaagggtgcccaggcaatcatcattaactctgattaccttttcgccacgctcgatgaggacgcatacaacgcgtacaccgcgaaggtcgcagcgcgtcacaagagggcgtacagatacatccatcatgccatggccaccaacaccatgttcgtcgccaagaacgatccgccatacgccaaggacctcgagagcaccgtctatctcgacccgcttgtgcgcgttgagccggataagaaggctggcacgtacgcgtacccggcaaagaagcccgctcagccatccaataaggagactgaggatgtgttgctgaagttaacccagtccaccgcgtcagctggcaccaacgtcggagtcgacgttgaggctttgtcggccatcccgatcgacaacgcgaccttcatcgagcgtaactacactcccgccgaaatcagttactgctcttcttctgctgatccccgggcaagctttgctggcacttggtgcgcgaaggaagctgttttcaagagcttgggcgttaaatccgacggcgcgggtgcggcgttgaaagaaatcgagatcgttcggaaatcgggcaagcccgaggtcgtgttctcgggcgttgcgaagcaacgggcggaagaaaagggagtcaaggacgtcagcgttagtatcagtcataacgaattccaggcggtcgctgttgctgttgcagagttggtttaa

SEQ ID NO: 62 - protein, 1854 aa, Lipomyces starkeyi
MARPETEQELAHILLVELLAYQFASPVRWIETQDVIFQEFNSERLVEIGPSPTLAGMAQRTLKAKYESYDAALSLQRQVLCYSKDAKEIYYTPDPVVVEAAPEPAAATAGAPAAAAVAAVAAAPAPSGPVAAVSDEPVKAVEILRSLVAQKLKKPYDQVPLAKAIKDLVGGKSTLQNEILGDLGKEFGSAPEKAEETPLEELGAAIQGSGFNGQLGKQSLSLIGRLVASKMPGGFNLTSTRKYLQDRWGLGPGRQDGVLLLAITIEPPARLAAEADAKKYLDEVAAKYASFAGISLSSGGGDAGAGGAGGGAIAIDSAAFEELTKDQTHLVRQQMELFAKYLKVDLRAGDKLFVDEVDASSELRKELDLWIAEHGDFYATGIVPSFSPLKARVYDSSWNWARQDALTMYYDIIFGRLSVVDREIVSHCILLMNRSNPTLLEFMQYHIDHCPEKRGDTYHLAKQLGQQLIDNCRDALGVEPVYKDVMYPTAPQTAIDVKGNIKYDEVPRVAVRKLEQYVKEMAAGGKITENRSRTKLHAGLARIYKIIRQQQKLSKSSKLQIKTLYEDVIQSLSLESGLANGSPSPDGTGRPTSPKRRKNGKGKKYTETIPFLHLKKKDMHGWDYSKPLTGIYLECLEQAAKSGISFKDKCALMTGAGAGSIGAAVLQGLLSGGAKVVVTTSRYSKEVTEYYQSIYAKYGASNSTLIVVPFNQGSKQDVDALVDYVYDTKKGLGWDLDYVIPFAAIPENGREIDGIDSKSELAHRMMLTNLLRILGNVKTQKLAHGYATRPAQVILPMSPNHGTFGSDGLYSESKLALETLFNRWYSESWGPYLTICGAVIGWTRGTGLMSQNNLIAERIEGLGVRTFSQQEMAFNILGLMSPAIVNLCQIEPVFADLNGGMQYIPNLKEASAQIRQELLQTSEIRRAVSAESAIEYKLVNGAEAERLQKSVVIQPRANIKFEFPRLKEYSEIAHLAEDLKGMVDLEKVVVVTGFAEVGPWGNARTRWEMEAYGQFSLEGCIEMAWIMGLIKHHNGQLKGKMYSGWVDTKSNEPVDDFDVKSKYEKHILEHSGIRLIEAELFDGYDPKKKKMLQEVVIEHDLEPFETSKETAYEFKREHGDKVEIFEIAETGQWTVRLLKGASLLIPKALQFDRLVAGQIPTGWDARRYGIGEDIISQVDPITLYVLVSTVEALLSAGVTDPYEFYKYVHVSEVGNCTGSGVGGMSALRGMYKDRFMDKPVQKDILQESFINTMSAWVNMLLLSSSGPIKTPVGACATSIESVDIGYETIISGKAKICLVGGYDDFQEEGSFEFGNMGATSNSETEIAHGRTPAEMSRPTTTTRAGFMESHGAGIQLIMTAKLALAMGVPVYGIIGMTATATDKIGRSVPAPGKGILTTARENRESKFPSPLLNIKYRRRQLEKRQSQIREWTESEILYLQQEVISMREQYENFDEREYLTERLAHVEREAARQEKDALAQWGNDFYKQDARIAPLRGALAVWNLTVDDLGVASFHGTSTVANDKNESMTIANMMTHLGRSKGNAILGIFQKYLTGHPKGAAGAWMLNGALQVLNTGLVPGNRNADNVDKVLEQYDSILYPSRSIQTDGIKAASVTSFGFGQKGAQAIIINSDYLFATLDEDAYNAYTAKVAARHKRAYRYIHHAMATNTMFVAKNDPPYAKDLESTVYLDPLVRVEPDKKAGTYAYPAKKPAQPSNKETEDVLLKLTQSTASAGTNVGVDVEALSAIPIDNATFIERNYTPAEISYCSSSADPRASFAGTWCAKEAVFKSLGVKSDGAGAALKEIEIVRKSGKPEVVFSGVAKQRAEEKGVKDVSVSISHNEFQAVAVAVAELV

(19) United States
(12) Patent Application Publication
(10) Pub. No.: US 2009/0049572 A1
(43) Pub. Date: Feb. 19, 2009

(54) ELEVATION OF OIL LEVELS IN PLANTS

(76) Inventors: Toni A. Voelker, Davis, CA (US); Dale L. Val, Woodland, CA (US); Thomas J. Savage, Sacramento, CA (US)

Correspondence Address: SONNENSCHEIN NATH & ROSENTHAL LLP, P.O. BOX 061080, SOUTH WACKER DRIVE STATION, SEARS TOWER, CHICAGO, IL, 60606, US

(21) Appl. No.: 12/181,855

(22) Filed: Jul. 29, 2008

Related U.S. Application Data
(63) Continuation-in-part of application No. 10/742,350, filed on Dec. 19, 2003, now Pat. No. 7,405,344.
(60) Provisional application No. 60/435,197, filed on Dec. 19, 2002.

(51) Int. Cl. C12N 15/82; C07H 21/04; A01H 5/00

(52) U.S. Cl. 800/281; 536/23.1; 536/24.5; 800/298; 800/312; 800/320; 800/320.2; 800/320.3; 800/320.1; 800/322; 800/314; 800/313

(57) ABSTRACT

This present invention provides a method for increasing oil levels in the tissues of plants by expression of a heterologous multifunctional fatty acid synthase (mfFAS) from Lipomyces starkeyi within the plant. In certain embodiments, the present invention provides isolated nucleic acid molecules encoding mfFAS enzymes from Lipomyces starkeyi for this purpose, and vectors and plants containing same.

This application is a continuation-in-part of U.S. patent application Ser. No. 10/742,350, filed Dec. 19, 2003, which application claims the priority of U.S. Provisional Patent Application No. 60/435,197, filed Dec. 19, 2002, the entire disclosures of which are incorporated herein by reference.

### BACKGROUND OF THE INVENTION

1. Field of the Invention

The present invention relates to the fields of nucleic acid chemistry and agricultural biotechnology. In particular, the present invention is directed at the identification of nucleic acids that encode proteins useful for increasing oil levels in plants and creating plants that include such nucleic acids.

2. Description of the Related Art

Plants such as Brassica sp. are a source of polyunsaturated oils. While tissues of most Brassica plant species contain little oil, the cultivation of certain plant types, over many acres, permit large quantities of Brassica plant oils to be produced. If the oil content of such plants could be increased, then plant oils could be produced more efficiently.

Higher plants such as Brassica synthesize fatty acids via a common metabolic pathway involving the co-factor ACP and the fatty acid synthase (FAS) enzyme complex. The FAS complex consists of about 8 separate enzymes that catalyze 30 or more individual reaction steps, all of which, in plants, are located in the plastids. In developing seeds, for example, where fatty acids are stored, the fatty acid synthase (FAS) enzyme complex is located in the plastids, synthesizes the fatty acids therein, and then the fatty acids are transported to the cytosol in accordance with energy needs there.

Certain workers have attempted to increase or modulate the oil content of plants. For example, U.S. Pat. No. 6,268,550 to Gengenbach et al., provides maize acetyl CoA carboxylase nucleic acids for altering the oil content of plants. Additionally, U.S. Pat. No. 5,925,805 to Ohlrogge et al., provides an Arabidopsis acetyl CoA carboxylase gene that can be used to increase the oil content of plants.

### SUMMARY OF THE INVENTION

A need exists for a method to increase the oil content of plants, including oilseed plants such as Brassica sp., and seeds. Moreover, it would be more energy efficient to provide the plant with a capability to synthesize fatty acids directly in the cytosol. Thus, in one embodiment, the invention provides an isolated nucleic acid sequence encoding a multifunctional fatty acid synthase, or its complement, selected from the group consisting of: (a) a nucleic acid sequence with at least about 85%, 90%, 95%, 98%, or 99% identity to SEQ ID NO:59 or SEQ ID NO:61; (b) a nucleic acid sequence encoding a polypeptide sequence with at least about 85%, 90%, 95%, 98%, or 99% identity to SEQ ID NO:60 or SEQ ID NO:62; (c) a nucleic acid sequence that hybridizes to SEQ ID NO:59 or SEQ ID NO:61 under conditions of 1×SSC, and 65° C. and encodes a polypeptide that displays multifunctional fatty acid synthase activity; and (d) the complement of (a)-(c).

The present invention also provides a plant comprising a heterologous nucleic acid encoding a multifunctional fatty acid synthase. In certain embodiments, the plant is a Brassica plant. In other embodiments, the plant is an oilseed plant. In particular embodiments, the plant is selected from the group consisting of: Brassica sp. including canola, mustard, crambe, oilseed rape, and rapeseed; Arabidopsis thaliana, soybean, safflower, sunflower, corn, rice, barley, millet, rye, wheat, oat, alfalfa, sorghum, soybean, grape, cotton, flax (linseed), castor bean, sesame, oil palm, jojoba, peanut, and Chinese tallow tree. In one embodiment, the plant further comprises a second nucleic acid encoding a phosphopantetheine:protein transferase. In certain embodiments the plant, such as a Brassica plant, produces increased oil levels in the seed tissue as a result of the multifunctional fatty acid synthase. In another embodiment the multifunctional fatty acid synthase is substantially located in the cytosol of the plant cell.

This present invention further provides a method for increasing oil levels in the tissues of a plant, such as Brassica sp., by expressing a gene encoding a multifunctional fatty acid synthase (mfFAS) on either a single or multiple polypeptide chains. In one embodiment of this present invention, the gene encodes a cytosol-targeted mfFAS. The source of the mfFAS may be selected from the group consisting of bacteria, fungi, plants, mycoplasma, and the like. In certain embodiments the source of the mfFAS is bacteria or fungi. In some embodiments the source of the mfFAS is a fungus. In particular embodiments of the invention, the source of the mfFAS is Lypomyces starkeyi. In certain embodiments, the nucleic acid molecule encoding the mfFAS from Lipomyces starkeyi comprises a nucleic acid sequence at least about 85%, 90%, 95%, 98%, or 99% identical to SEQ ID NO:59 or SEQ ID NO:61. An isolated nucleic acid comprising a nucleic acid sequence at least about 85%, 90%, 95%, 98%, or 99% identical to SEQ ID NO:59 or SEQ ID NO:61 therefore forms an embodiment of the present invention. Another embodiment of the invention comprises a nucleic acid sequence that encodes a mfFAS protein at least about 85%, 90%, 95%, 98%, or 99% identical to SEQ ID NO:60 or SEQ ID NO:62. In particular embodiments the mfFAS comprises SEQ ID NO:60 or SEQ ID NO:62, or a multifunctional fatty acid synthase comprising a sequence at least about 85%, 90%, 95%, 98%, or 99% identical to SEQ ID NO:60 or SEQ ID NO:62. In another embodiment of this present invention, the expression of the mfFAS gene is in the seed tissue of a plant, such as a Brassica plant, preferably resulting in the accumulation of oil in the seed.

In another embodiment the present invention provides plant transformation vectors containing a mfFAS gene. Transformed plants and seeds, such as Brassica plants, are also provided.

In yet another embodiment, the present invention provides a method of producing a plant oil, comprising the steps of: a) growing a plant such as an oilseed, the genome of which contains a nucleic acid molecule (i) encoding a multifunctional fatty acid synthase comprising a sequence at least about 85%, 90%, 95%, 98%, or 99% identical to SEQ ID NO:60 or SEQ ID NO:62; or (ii) a nucleic acid that encodes an mfFAS and which is at least about 85%, 90%, 95%, 98%, or 99% identical to SEQ ID NO:59 or SEQ ID NO:61, to produce oil-containing seeds; and b) extracting oil from the seeds. In particular embodiments, the nucleic acid comprises SEQ ID NO:59 or SEQ ID NO:61. In other embodiments the mfFAS comprises SEQ ID NO:60 or SEQ ID NO:62. In another embodiment, the present invention provides a method of producing a plant oil, comprising the steps of: a) growing an oilseed plant, the genome of which contains a nucleic acid molecule encoding a phosphopantetheine:protein transferase, to produce oil-containing seeds; and b) extracting oil from the seeds. In particular embodiments, the plant is a Brassica plant.

### DESCRIPTION OF THE FIGURES

FIG. 1 provides a map of the plasmid pMON70058 that contains the 8 KB fasA gene from Brevibacterium ammoniagenes.

FIG. 2A-2J provides an alignment of the fasA Brevibacterium ammoniagenes nucleic acid sequence (SEQ ID NO: 1) provided herein with a published fasA Brevibacterium ammoniagenes nucleic acid sequence (Stuible et al., J. Bacteriol., 178:4787, 1996). A number of differences at the DNA level were observed.

FIG. 3A-3D provides an alignment of the fasA Brevibacterium ammoniagenes amino acid sequence (SEQ ID NO: 2) provided herein with a published fasA Brevibacterium ammoniagenes amino acid sequence (Stuible et al., J. Bacteriol., 178:4787, 1996). A number of differences at the protein level were observed.

FIG. 4 illustrates FasA enzyme activity of the cloned fasA gene from B. ammoniagenes. The FasA enzyme activity was determined as outlined in Kawaguchi et al., (Methods in Enzymology, 71:120-127, 1981) for partially purified enzyme preparations from B. ammoniagenes (B.a.), for an untransformed E. coli strain VCS257 (E.c.), and for the same strain transformed with the pptl expressing plasmid (E.c.+P), the fasA cosmid (E.c.+FA), or the pptl expressing plasmid and the fasA cosmid (E.c.+P+FA).

FIG. 5 provides a schematic representation of the preparation of pMON75201 as well as a map of pMON75201.

FIG. 6 shows the results of the analyses of R2 seed from events generated from the transformation of canola explants with the vector pMON75201.

FIG. 7 shows the statistical analysis of the oil results from positive and negative isolines of event BN_G1216.

### BRIEF DESCRIPTION OF THE SEQUENCES

SEQ ID NO: 1 is a DNA encoding FasA from Brevibacterium ammoniagenes.

SEQ ID NO: 2 is a protein known as FasA from Brevibacterium ammoniagenes.

SEQ ID NO: 3 is a DNA encoding a phosphopantetheine:protein transferase (PPT1) enzyme from B. ammoniagenes.

SEQ ID NO: 4 is a protein known as phosphopantetheine:protein transferase (PPT1) enzyme from B. ammoniagenes.

SEQ ID NOs: 5-14 are nucleic acids used as PCR primers.

SEQ ID NO: 15 is a protein known as fatty acid synthase 1 of Schizosaccharomyces pombe; NCBI Accession No. CAB54157.

SEQ ID NO: 16 is a DNA encoding fatty acid synthase subunit beta of Schizosaccharomyces pombe.

SEQ ID NO: 17 is a protein known as fatty acid synthase subunit alpha of Schizosaccharomyces pombe; NCBI Accession No. D83412.

SEQ ID NO: 18 is a protein known as fatty acid synthase subunit beta of Saccharomyces cerevisiae; NCBI Accession No. CAA82025.

SEQ ID NO: 19 is a protein known as fatty acid synthase subunit alpha of Saccharomyces cerevisiae; NCBI Accession No. CAA97948.

SEQ ID NO: 20 is a protein known as fatty acid synthase subunit beta of Candida albicans; NCBI Accession No. CAA52907.

SEQ ID NO: 21 is a DNA encoding fatty acid synthase subunit alpha of Candida albicans; NCBI Accession No. L29063.

SEQ ID NO: 22 is a protein known as fatty acid synthase subunit alpha of Candida albicans; NCBI Accession No. L29063.

SEQ ID NO: 23 is a protein known as fatty acid synthase of Mycobacterium 10 tuberculosis H37Rv; NCBI Accession No. CAB06201.

SEQ ID NO: 24 is a protein known as fatty acid synthase of Mycobacterium leprae; NCBI Accession No. CAB39571.

SEQ ID NO: 25 is a protein known as fatty acid synthase of Caenorhabditis elegans; NCBI Accession No. NP492-417.

SEQ ID NO: 26 is a DNA encoding fatty acid synthase (FAS) of Rattus norvegicus; NCBI Accession No. X13415.

SEQ ID NO: 27 is a protein known as fatty acid synthase (FAS) of Rattus norvegicus; NCBI Accession No. X13415.

SEQ ID NO: 28 is a DNA encoding fatty acid synthase (FAS) of chicken (Gallus 20 gallus); NCBI Accession No. J03860 M22987.

SEQ ID NO: 29 is a protein known as fatty acid synthase (FAS) of chicken (Gallus gallus); NCBI Accession No. J03860 M22987.

SEQ ID NO: 30 is a DNA encoding fatty acid synthase (FAS) of Mycobacterium bovis; NCBI Accession No. U36763.

SEQ ID NO: 31 is a protein known as fatty acid synthase (FAS) of Mycobacterium bovis; NCBI Accession No. U36763.

SEQ ID NO: 32 is a DNA encoding a phosphopantetheine:protein transferase (sfp gene product) enzyme from Bacillus subtilis; NCBI Accession No. X63158.

SEQ ID NO: 33 is a protein known as phosphopantetheine:protein transferase (sfp gene product) enzyme from Bacillus subtilis; NCBI Accession No. X63158.

SEQ ID NO: 34 is a DNA encoding a phosphopantetheine:protein transferase (gsp gene product) enzyme from Brevibacillus brevis (ATCC 9999); NCBI Accession No. X76434.

SEQ ID NO: 35 is a protein known as phosphopantetheine:protein transferase (gsp gene product) enzyme from Brevibacillus brevis (ATCC 9999); NCBI Accession No. X76434.

SEQ ID NO: 36 is a DNA encoding a phosphopantetheine:protein transferase (entD gene product) enzyme from Escherichia coli; NCBI Accession No. D90700.

SEQ ID NO: 37 is a protein known as phosphopantetheine:protein transferase (entD gene product) enzyme from Escherichia coli; NCBI Accession No. D90700.

SEQ ID NO: 38 is a DNA encoding a phosphopantetheine:protein transferase (ppta gene product) enzyme from Streptomyces verticillus; NCBI Accession No. AF210311.

SEQ ID NO: 39 is a protein known as phosphopantetheine:protein transferase (pptA gene product) enzyme from Streptomyces verticillus; NCBI Accession No. AF210311.

SEQ ID NO: 40 is a DNA encoding an α-aminoadipate reductase small subunit (lys5 gene product) enzyme from Saccharomyces cerevisiae; NCBI Accession No. U32586.

SEQ ID NO: 41 is a protein known as the small subunit (lys5 gene product) of an α-aminoadipate reductase from Saccharomyces cerevisiae; NCBI Accession No. U32586.

SEQ ID NO: 42 is a DNA encoding an open reading frame o195 from Escherichia coli; NCBI Accession No. U00039.

SEQ ID NO: 43 is a protein encoded by open reading frame o195 from Escherichia coli; NCBI Accession No. U00039.

SEQ ID NOs: 44-47 are a synthetic peptide fragment sequences.

SEQ ID NO: 48-49 are nucleic acids used as PCR primers.

SEQ ID NO:50 is the LsFAS1 537 5′ race primer.

SEQ ID NO:51 is the LsFAS lend with stop primer.

SEQ ID NO:52 is the LsFAS2gfpATG primer.

SEQ ID NO:53-58 are nucleic acids used as PCR primers.

SEQ ID NO:59 is a nucleotide sequence encoding FAS I from Lipomyces starkeyi.

SEQ ID NO:60 is a polypeptide sequence encoding Ls FAS I from Lipomyces starkeyi.

SEQ ID NO:61 is a nucleotide sequence encoding FAS II from Lipomyces starkeyi.

SEQ ID NO:62 is a polypeptide sequence encoding Ls FAS II from Lipomyces starkeyi.

### DEFINITIONS

The following definitions are provided as an aid to understanding the detailed description of the present invention.

The phrases “coding sequence,” “coding region,” “structural sequence,” and “structural nucleic acid sequence” refer to a physical structure comprising an orderly arrangement of nucleotides. The nucleotides are arranged in a series of triplets that each form a codon. Each codon encodes a specific amino acid. Thus, the coding sequence, coding region, structural sequence, and structural nucleic acid sequence encode a series of amino acids forming a protein, polypeptide, or peptide sequence. The coding sequence, coding region, structural sequence, and structural nucleic acid sequence may be contained within a larger nucleic acid molecule, vector, or the like. In addition, the orderly arrangement of nucleotides in these sequences may be depicted in the form of a sequence listing, figure, table, electronic medium, or the like.

The phrases “DNA sequence,” “nucleic acid sequence,” and “nucleic acid molecule” refer to a physical structure comprising an orderly arrangement of nucleotides. The DNA sequence or nucleotide sequence may be contained within a larger nucleotide molecule, vector, or the like. In addition, the orderly arrangement of nucleic acids in these sequences may be depicted in the form of a sequence listing, figure, table, electronic medium, or the like.

The term “expression” refers to the transcription of a gene to produce the corresponding mRNA and translation of this mRNA to produce the corresponding gene product (i.e., a peptide, polypeptide, or protein).

The phrase “expression of antisense RNA” refers to the transcription of a DNA to produce a first RNA molecule capable of hybridizing to a second RNA molecule, which second RNA molecule encodes a gene product that is desirably down-regulated.

The term “homology” refers to the level of similarity between 2 or more nucleic acid or amino acid sequences in terms of percent of positional identity (i.e., sequence similarity or identity). Homology also refers to the concept of similar functional properties among different nucleic acids or proteins.

The term “heterologous” refers to the relationship between 2 or more nucleic acid or protein sequences that are derived from different sources. For example, a promoter is heterologous with respect to a coding sequence if such a combination is not normally found in nature. In addition, a particular nucleic acid molecule may be “heterologous” with respect to a cell or organism into which it is inserted (i.e., does not naturally occur in that particular cell or organism).

The term “hybridization” refers to the ability of a first strand of nucleic acid to join with a second strand via hydrogen bond base pairing when the 2 nucleic acid strands have sufficient sequence complementarity. Hybridization occurs when the 2 nucleic acid molecules anneal to one another under appropriate conditions.

The phrase “operably linked” refers to the functional spatial arrangement of 2 or more nucleic acid regions or nucleic acid sequences. For example, a promoter region may be positioned relative to a nucleic acid sequence such that transcription of the nucleic acid sequence is directed by the promoter region. Thus, a promoter region is operably linked to the nucleic acid sequence.

In the context of the present invention, the terms “plant” or “plants” refer to Brassica sp. plants.

The terms “promoter” or “promoter region” refers to a nucleic acid sequence, usually found upstream (5′) to a coding sequence, that is capable of directing transcription of a nucleic acid sequence into mRNA. The promoter or promoter region typically provides a recognition site for RNA polymerase and the other factors necessary for proper initiation of transcription. As contemplated herein, a promoter or promoter region includes variations of promoters derived by inserting or deleting regulatory regions, subjecting the promoter to random or site-directed mutagenesis, and the like. The activity or strength of a promoter may be measured in terms of the amounts of RNA it produces, or the amount of protein accumulation in a cell or tissue, relative to a second promoter that is similarly measured.

The term “5′-UTR” refers to the untranslated region of DNA upstream, or 5′, of the coding region of a gene.

The term “3′-UTR” refers to the untranslated region of DNA downstream, or 3′, of the coding region of a gene.

The phrase “recombinant vector” refers to any agent by or in which a nucleic acid of interest is amplified, expressed, or stored, such as a plasmid, cosmid, virus, autonomously replicating sequence, phage, or linear single-stranded, circular single-stranded, linear double-stranded, or circular double-stranded DNA or RNA nucleotide sequence. The recombinant vector may be derived from any source and is capable of genomic integration or autonomous replication.

The phrase “regulatory sequence” refers to a nucleotide sequence located upstream (5′), within, or downstream (3′) with respect to a coding sequence. Transcription and expression of the coding sequence is typically impacted by the presence or absence of the regulatory sequence.

The phrase “substantially homologous” refers to 2 sequences that are at least about 90% identical in sequence, as measured by the CLUSTAL W method in the Omiga program, using default parameters (Version 2.0; Accelrys, San Diego, Calif.).

The term “transformation” refers to the introduction of nucleic acid into a recipient host. The term “host” refers to bacteria cells, fungi, animals or animal cells, plants or seeds, or any plant parts or tissues including plant cells, protoplasts, calli, roots, tubers, seeds, stems, leaves, seedlings, embryos, and pollen.

As used herein, the phrase “transgenic Brassica plant” refers to a Brassica plant having an introduced nucleic acid stably introduced into a genome of that plant, for example, the nuclear or plastid genomes.

As used herein, the phrase “substantially purified” refers to a molecule separated from substantially all other molecules normally associated with it in its native state. More preferably, a substantially purified molecule is the predominant species present in a preparation. A substantially purified molecule may be greater than about 60% free, preferably about 75% free, more preferably about 90% free, and most preferably about 95% free from the other molecules (exclusive of solvent) present in the natural mixture. The phrase “substantially purified” is not intended to encompass molecules present in their native state.

### DETAILED DESCRIPTION OF THE INVENTION

The present invention provides a multifunctional fatty acid synthase (“mfFAS”) that encodes the enzymatic functions required to synthesize palmitoyl (16:0) CoA, stearoyl (18:0) CoA, and oleoyl (18:1) CoA, the fatty acids used as precursors for other long chain saturated and unsaturated fatty acids. Obtaining nucleic acid sequences capable of producing increased oil content in Brassica plants is problematic because many non-associated, monofunctional enzymes are used to make fatty acids in Brassica plants. Accordingly, cloning and genetic manipulation of plant fatty acid synthases (“FASs”) would require isolation and coordinated expression of at least 8 separate genes. In particular, plant fatty acid synthesis depends on availability of the following plastid-localized FAS enzymes: Malonyl-CoA:ACP transacylase, β-ketoacyl-ACP synthase III, β-ketoacyl-ACP synthase I, β-ketoacyl-ACP synthase II, β-ketoacyl-ACP reductase, β-hydroxyacyl-ACP dehydratase, enoyl-ACP reductase, and stearoyl-ACP desaturase. For movement of the end-product, acyl-ACP, from the plastid to the cytosol of the cell, two more enzymatic activities are required: acyl-ACP thioesterase and acyl-CoA synthase.

However, the present invention solves this problem by providing a multifunctional fatty acid synthase that encodes all of the FAS enzymatic functions in a single, long polypeptide chain or in two chains that combine together, which may be employed in the cytosol or plastid, preferably in both, more preferably in the cytosol. Such a multifunctional fatty acid synthase is surprisingly effective in Brassica plants even though its structure is so dissimilar from plant endogenous fatty acid synthases. Most preferably, the mfFAS of the present invention is employed in the cytosol of a Brassica plant, and in this way the need for an acyl carrier protein (“ACP”) in fatty acid synthesis and the enzymes acyl-ACP thioesterase and acyl-CoA synthase is removed. Accordingly, not only does the present invention remove the need to clone at least 8 different genes to accomplish altered fatty acid synthesis in a Brassica plant, but when the mfFAS is employed in the cytosol, it replaces the function of 11 different plant gene products.

Fatty Acid Synthases:

Fatty acid synthases are among the functionally most complex multienzyme systems known, which can be formed from a single polypeptide or multiple polypeptides. For mfFASs formed of a single polypeptide, there are multiple regions included thereon that perform the various enzymatic activities; such regions are referred to as “domains.” Multifunctional fatty acid synthases formed of multiple polypeptides include various FAS domains as well, which require the interaction of the constituent polypeptides to function. Such polypeptides, whether a multi-domain or single-domain polypeptide, can be isolated from an organism, or can be generated by combining domains or parts of domains together at the nucleic acid level using conventional recombinant technology. Accordingly, recombinant chimeric nucleic acids that combine some mfFAS domains from one source with the remainder of the mfFAS domains from one or more second sources are preferred embodiments of the present invention. In the same fashion, the nucleic acid sequences in a mfFAS gene that encodes a particular domain can be replaced with a homologous nucleic acid sequence from a second source that encodes the same domain.

Fatty acid synthases usually comprise a set of 8 different functional domains and catalyze more than about 30 individual reaction steps. Two structurally distinct classes of fatty acid synthases exist. Type I fatty acid synthases are multifunctional synthases, commonly found in non-plant eukaryotes and in a few bacterial species. Type H fatty acid synthases constitute a set of separate, monofunctional polypeptides that are found in most bacteria and in the plastids of higher plants. These polypeptides must properly assemble into a multimeric complex before the synthase becomes active. The fatty acid synthase from some bacteria, such as Brevibacterium ammoniagenes, is unlike plant and animal synthases in that it has a ninth catalytic activity (Seyama and Kawaguchi (1987), in Dolthin et al., (eds.), Pyridine Nucleotide Coenzymes: Chemical, Biochemical and Medical Aspects, vol. 2B, Wiley, NY, pp. 381-431), the 3-hydroxydecanoyl β,y-dehydratase, which enables synthesis of both saturated and unsaturated fatty acids.

For transgenic purposes, type I “multifunctional fatty acid synthases” may have certain advantages over the type II “monofunctional” fatty acid synthases. For example, the type I multifunctional fatty acid synthases may have greater stability and/or better-coordinated expression. Addition of a single polypeptide specific for one of the enzymatic fatty acid synthase activities to a plant by transgenic means may not provide overproduction of the entire fatty acid synthase complex because there may not be sufficient endogenous amounts of the other non-transgenic FAS polypeptides to substantially increase levels of the functional complex. In contrast, nucleic acids encoding a type I multifunctional fatty acid synthase can reliably be used to overproduce all of the enzymatic functions of fatty acid synthase.

According to the present invention, nucleic acids encoding one or more of the separate domains from a type II monofunctional fatty acid synthase can be fused or linked to provide a synthetic multifunctional fatty acid synthase that can generate high oil levels when expressed within a host, such as, for example, a Brassica plant cell, plant tissue, or seed. Such a fused, synthetic multifunctional fatty acid synthase can be made by fusing or linking the separate enzymatic functions associated with the various polypeptides of type II fatty acid synthases by chemically linking the nucleic acids that encode the various polypeptides. The overall sequence of such a synthetic gene generally aligns with that of a type I multifunctional fatty acid synthase. Using such sequence alignments, the spacing and orientation of polypeptides that contain the various fatty acid synthase activities can be adjusted or modified by altering the lengths of linking DNA between coding regions to generate a synthetic multifunctional fatty acid synthase DNA construct that optimally aligns with a natural type I multifunctional fatty acid synthase gene.

The fatty acid synthase polypeptides of the present invention can therefore encode more than one of the enzymes associated with fatty acid synthase, such as, for example, 2 through and including 9, thereby enabling up to the same 9 catalytic activities as are found in the mfFAS of Brevibacterium ammoniagenes. Any of the enzymes involved in the various steps of fatty acid synthesis can be joined. The first step in initiation stage of fatty acid synthesis is the carboxylation of the 2-carbon acetyl-CoA to form the 3-carbon P-ketoacid malonyl-CoA by acetyl-CoA carboxylase (ACCase). The ACCase step is irreversible, so once this step is accomplished, the resultant carbon compound is committed to fatty acid synthesis. All subsequent steps are catalyzed by the FAS. Malonyl-ACP is synthesized from malonyl-CoA and ACP by the enzyme malonyl-CoA:ACP transacylase. An acetyl moiety from acetyl-CoA is joined to a malonyl-ACP in a condensation reaction catalyzed by β-ketoacyl-ACP synthase III. Elongation of acetyl-ACP to 16- and 18-carbon fatty acids involves the cyclical action of the following sequence of reactions. After acetyl-CoA is condensed with malonyl-ACP using β-ketoacyl-ACP synthase, a β-ketoacyl-ACP is formed. The keto group on the β-ketoacyl-ACP is then reduced to an alcohol by β-ketoacyl-ACP reductase. The alcohol is removed in a dehydration reaction to form an enoyl-ACP by β-hydroxyacyl-ACP dehydratase. Finally, the enoyl-ACP is reduced to form the elongated saturated acyl-ACP by enoyl-ACP reductase.

The enzyme β-ketoacyl-ACP synthase I catalyzes elongation up to palmitoyl-ACP (C16:0), which is generally the end product from which other types of fatty acids are made. The enzyme β-ketoacyl-ACP synthase H catalyzes the final elongation of palmitoyl-ACP to stearoyl-ACP (C18:0).

Common plant unsaturated fatty acids, such as oleic, linoleic, and ct-linolenic acids, originate from the desaturation of stearoyl-ACP to form oleoyl-ACP (C18:1) in a reaction catalyzed by a soluble plastid enzyme, Δ-9 desaturase (also often referred to as “stearoyl-ACP desaturase”). Molecular oxygen is required for desaturation and reduced ferredoxin serves as an electron co-donor.

Hence, the present invention contemplates polypeptides encoding several functions, for example, those relating to acyl carrier protein, malonyl CoA-ACP acyltransferase, β-ketoacyl-ACP synthase III, β-ketoacyl-ACP reductase, β-hydroxyacyl-ACP dehydratase, enoyl-ACP reductase, β-ketoacyl-ACP synthase I, β-ketoacyl-ACP synthase II, and Δ-9 desaturase.

In one embodiment, the present invention provides an isolated mfFAS polypeptide from a species of the group consisting of Brevibacterium ammoniagenes, Schizosaccharomyces pombe, Saccharomyces cerevesiae, Candida albicans, Mycobacterium tuberculosis, Caenorhabditis elegans, Rattus norvegicus, Gallus gallus, Lipomyces starkeyi, Rhodosporidium toruloides, and Mycobacterium bovis. Preferably, the mfFAS polypeptide is isolated from Brevibacterium ammoniagenes, Schizosaccharomyces pombe, Saccharomyces cerevesiae, Candida albicans; more preferably, the mfFAS polypeptides is isolated from Brevibacterium ammoniagenes. Such mfFAS polypeptides include one selected from the group consisting of SEQ ID NOs: 2, 15, 17, 18, 19, 20, 22, 23, 24, 25, 27, 29, and 31. Preferably, the mfFAS polypeptide used in the context of the present invention is one selected from the group consisting of SEQ ID NOs: 2, 15, 17, 18, 19, 20, and 22; more preferably, the mfFAS is SEQ ID NO: 2. Any of the aforementioned mfFAS polypeptides functions to increase the oil content of Brassica plant tissues.

mfFAS Nucleic Acids: The present invention uses nucleic acids that encode multifunctional fatty acid synthases, which are used in the context of the present invention for increasing the oil content of Brassica plant tissues. Such nucleic acids can encode a type I multifunctional fatty acid synthase that has been isolated from an organism. Preferred organisms from which nucleic acids encoding mfFAS can be isolated include, without limitation: bacteria, preferably Brevibacteria and Bacilli; fungi, preferably Saccharomycetes, Schizosaccharomycetes, Lipomyces starkeyi, Rhodosporidium toruloides, or Candidae; mycobacteria; nematodes, preferably Caenorhabdites; and mammals, preferably rat or chicken. Alternatively, the nucleic acids can encode a multifunctional fatty acid synthase that has been recombinantly generated to contain a fusion of 2 or more regions that encode monofunctional enzymatic domains that facilitate 2 or more of the steps required to make a fatty acid.

In one embodiment, the present invention uses an isolated nucleic acid that encodes a protein having mfFAS activity, which nucleic acid is selected from the group consisting of SEQ ID NOs: 1, 16, 21, 26, 28, 30, 59, and 61, and complements thereof, and nucleic acids having at least about 70% sequence identity thereof. In certain embodiments the nucleic acid comprises SEQ ID NO: 1, 16, 21, 26, 28, 30, 59, or 61; in particular embodiments the nucleic acid comprises SEQ ID NO: 59 or SEQ ID NO:61. The percent sequence identity of included nucleic acids in the group is preferably at least about 75%, more preferably at least about 80%, yet more preferably at least about 85%, and yet more preferably at least about 90%; even more preferably at least about 95%; and most preferably at least about 98%. The nucleic acids of the present invention can be isolated from any species that has a multifunctional fatty acid synthase, including without limitation Brevibacterium ammoniagenes (source of SEQ ID NO: 1), Schizosaccharomyces pombe (source of SEQ ID NO: 16), Saccharomyces cerevesiae, Candida albicans (source of SEQ ID NO: 21), Mycobacterium tuberculosis, Mycobacterium leprae, Mycobacterium bovis (source of SEQ ID NO: 30), Caenorhabditis elegans, rat (source of SEQ ID NO: 26), chicken (source of SEQ ID NO: 28), and Lipomyces starkeyi (source of SEQ ID NO:59 and SEQ ID NO:61). In particular embodiments, the present invention provides a nucleic acid that encodes mfFAS from Lipomyces starkeyi.

In yet another embodiment, the present invention uses a nucleic acid that encodes a multifunctional fatty acid synthase having an amino acid sequence comprising a polypeptide selected from the group consisting of SEQ ID NOs: 2, 15, 17, 18, 19, 20, 22, 23, 24, 25, 27, 29, 31, 59 and 61. In certain embodiments, the polypeptide comprises SEQ ID NO:60 or SEQ ID NO:62. The present invention also uses the set of nucleic acids that includes those nucleic acids that are at least about 80% identical to those that encode SEQ ID NO: 2, 15, 17, 18, 19, 20, 22, 23, 24, 25, 27, 29, 31, 59, or 61. In some embodiments the set of nucleic acids are at least about 85% identical to one or more of the nucleic acids that encode one or more of the above identified SEQ ID NOs; in particular embodiment, at least about 90% identical, at least about 95% identical; or at least about 98% identical.

The present invention also uses vectors containing such multifunctional fatty acid synthase nucleic acids. As set forth in further detail hereinbelow, preferred nucleic acids include appropriate regulatory elements operably linked thereto that facilitate efficient expression of the inventive nucleic acids in a host, including without limitation, Brassica plant hosts. Vectors useful in the context of the present invention can include such regulatory elements.

In a preferred embodiment of the present invention, the nucleic acid molecules of the present invention encode enzymes that are allelic to those defined. As used herein, a mutant enzyme is any enzyme that contains an amino acid that is different from the amino acid in the same position of an enzyme of the same type.

The nucleic acids and vectors described herein need not have the exact nucleic acid sequences described herein. Instead, the sequences of these nucleic acids and vectors can vary, so long as the nucleic acid either performs the function for which it is intended or has some other utility, for example, as a nucleic acid probe for complementary nucleic acids. For example, some sequence variability in any part of a multifunctional fatty acid synthase nucleic acid is permitted so long as the mutant or variant polypeptide or polypeptides retains at least about 10% of the fatty acid synthase (FasA) activity observed under similar conditions for an analogous wild type fatty acid synthase enzyme, including when the polypeptide(s) retain at least about 25% of the FasA activity; at least about 50% of the FasA activity; at least about 75% of the FasA activity; and at least about 90% of the FasA activity. In certain embodiments the aforementioned sequence variability results in increased FasA activity. In particular embodiments, the comparison of enzymatic activity is with the wild type Brevibacterium ammoniagenes fatty acid synthase (SEQ ID NO: 2).

Fragment and variant nucleic acids, for example of SEQ ID NO: 59 or 61, are also encompassed by the present invention. Nucleic acid “fragments” encompassed by the present invention are of 3 general types. First, fragment nucleic acids that are not full length but do perform their intended function (fatty acid synthesis) are encompassed within the present invention. Second, fragments of nucleic acids identified herein that are useful as hybridization probes, but generally are not functional for fatty acid synthesis, are also included in the present invention. And, third, fragments of nucleic acids identified herein can be used in suppression technologies known in the art, such as, for example, anti-sense technology or RNA inhibition (RNAi), which provides for reducing carbon flow in a plant into oil, making more carbon available for protein or starch accumulation, for example. Thus, fragments of a nucleotide sequence, such as SEQ ID NO: 1, 16, 21, 26, 28, 30, 59, or 61, without limitation, may range from at least about 15 nucleotides, at least about 17 nucleotides, at least about 18 nucleotides, at least about 20 nucleotides, at least about 50 nucleotides, at least about 100 nucleotides, or more. In general, a fragment nucleic acid of the present invention can have any upper size limit so long as it is related in sequence to the nucleic acids of the present invention but does not include the full length.

In another embodiment, the present invention provides DNA molecules comprising a sequence encoding a consensus amino acid sequence, and complements thereof. In another aspect, the present invention provides DNA molecules comprising a sequence encoding a polypeptide comprising a conserved fragment of an amino acid consensus sequence. The present invention includes the use of consensus sequence and fragments thereof in transgenic Brassica plants, other organisms, and for other uses including those described below.

As used herein, “variants” have substantially similar or substantially homologous sequences when compared to reference or wild type sequence. For nucleotide sequences that encode proteins, variants also include those sequences that, because of the degeneracy of the genetic code, encode the identical amino acid sequence of the reference protein. Variant nucleic acids also include those that encode polypeptides that do not have amino acid sequences identical to that of the proteins identified herein, but which encode an active protein with conservative changes in the amino acid sequence.

As is known by one of skill in the art, the genetic code is “degenerate,” meaning that several trinucleotide codons can encode the same amino acid. This degeneracy is apparent from Table 1.

TABLE 1
Degeneracy of genetic code.

| | 2^{nd} Position | | | | |
|---|---|---|---|---|---|
| 1^{St} Position | T | C | A | G | 3^{rd} Position |
| T | TTT = Phe | TCT = Ser | TAT = Tyr | TGT = Cys | T |
| T | TTC = Phe | TCC = Ser | TAC = Tyr | TGC = Cys | C |
| T | TTA = Leu | TCA = Ser | TAA = Stop | TGA = Stop | A |
| T | TTG = Leu | TCG = Ser | TAG = Stop | TGG = Trp | G |
| C | CTT = Leu | CCT = Pro | CAT = His | CGT = Arg | T |
| C | CTC = Leu | CCC = Pro | CAC = His | CGC = Arg | C |
| C | CTA = Leu | CCA = Pro | CAA = Gln | CGA = Arg | A |
| C | CTG = Leu | CCG = Pro | CAG = Gln | CGG = Arg | G |
| A | ATT = Ile | ACT = Thr | AAT = Asn | AGT = Ser | T |
| A | ATC = Ile | ACC = Thr | AAC = Asn | AGC = Ser | C |
| A | ATA = Ile | ACA = Thr | AAA = Lys | AGA = Arg | A |
| A | ATG = Met | ACG = Thr | AAG = Lys | AGG = Arg | G |
| G | GTT = Val | GCT = Ala | GAT = Asp | GGT = Gly | T |
| G | GTC = Val | GCC = Ala | GAC = Asp | GGC = Gly | C |
| G | GTA = Val | GCA = Ala | GAA = Gln | GGA = Gly | A |
| G | GTG = Val | GCG = Ala | GAG = Gin | GGG = Gly | G |

Hence, many changes in the nucleotide sequence of the variant may be silent and may not alter the amino acid sequence encoded by the nucleic acid. Where nucleic acid sequence alterations are silent, a variant nucleic acid will encode a polypeptide with the same amino acid sequence as the reference nucleic acid. Therefore, a particular nucleic acid of the present invention also encompasses variants with degenerate codon substitutions, and complementary sequences thereof, as well as the sequence explicitly specified by a SEQ ID NO as set forth herein. Specifically, degenerate codon substitutions may be achieved by generating sequences in which the reference codon is replaced by any of the codons for the amino acid specified by the reference codon. In general, the third position of one or more selected codons can be substituted with mixed-base and/or deoxyinosine residues as disclosed by Batzer et al., Nucleic Acid Res., 19:5081 (1991) and/or Ohtsuka et al., J. Biol. Chem., 260:2605 (1985); Rossolini et al., Mol. Cell. Probes, 8:91 (1994).

A host cell often displays a preferred pattern of codon usage. Structural nucleic acid sequences are preferably constructed to utilize the codon usage pattern of the particular host cell. This generally enhances the expression of the structural nucleic acid sequence in a transformed host cell. Any disclosed nucleic acid or amino acid sequence may be modified to reflect the preferred codon usage of a host cell or organism in which they are contained.

Modification of a structural nucleic acid sequence for optimal codon usage in plants is described in U.S. Pat. No. 5,689,052, which is incorporated herein by reference. In a preferred embodiment, the present invention includes nucleic acids that encode mfFAS and that are codon-optimized in a Brassica plant. In a preferred embodiment the plants are of the Brassica species, and most preferably Brassica napus (canola).

However, the present invention is not limited to silent changes in the present nucleotide sequences but also includes variant nucleic acid sequences that conservatively alter the amino acid sequence of a polypeptide of the present invention. Because it is the interactive capacity and nature of a protein that defines that protein's biological functional activity, certain amino acid sequence substitutions can be made in a protein sequence and, of course, its underlying DNA coding sequence and, nevertheless, a protein with like properties can still be obtained. It is thus contemplated by the inventors that various changes may be made in the peptide sequences of the proteins or fragments of the present invention, or corresponding DNA sequences that encode the peptides, without appreciable loss of their biological utility or activity. According to the present invention, then, variant and reference nucleic acids of the present invention may differ in the encoded amino acid sequence by one or more substitutions, additions, insertions, deletions, fusions, and truncations, which may be present in any combination, so long as an active mfFAS protein is encoded by the variant nucleic acid. Such variant nucleic acids will not encode exactly the same amino acid sequence as the reference nucleic acid, but have conservative sequence changes. It is known that codons capable of coding for such conservative amino acid substitutions are known in the art.

Another approach to identifying conservative amino acid substitutions require analysis of the hydropathic index of amino acids may be considered. The importance of the hydropathic amino acid index in conferring interactive biological function on a protein is generally understood in the art (Kyte and Doolittle, J. Mol. Biol., 157:105-132, 1982). It is accepted that the relative hydropathic character of the amino acid contributes to the secondary structure of the resultant polypeptide, which in turn defines the interaction of the protein with other molecules, for example, enzymes, substrates, receptors, DNA, antibodies, antigens, and the like.

Each amino acid has been assigned a hydropathic index on the basis of its hydrophobicity and charge characteristics (Kyte and Doolittle, J. Mol. Biol., 157:105-132, 1982); these are isoleucine (+4.5), valine (+4.2), leucine (+3.8), phenylalanine (+2.8), cysteine/cystine (+2.5), methionine (+1.9), alanine (+1.8), glycine (−0.4), threonine (−0.7), serine (−0.8), tryptophan (−0.9), tyrosine (−1.3), proline (−1.6), histidine (−3.2), glutamate (−3.5), glutamine (−3.5), aspartate (−3.5), asparagine (−3.5), lysine (−3.9), and arginine (−4.5).

In making such changes, the substitution of amino acids whose hydropathic indices are within ±2 is preferred, those that are within ±1 are particularly preferred, and those within ±0.5 are even more particularly preferred.

It is also understood in the art that the substitution of like amino acids can be made effectively on the basis of hydrophilicity. U.S. Pat. No. 4,554,101, states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0), lysine (+3.0), aspartate (+3.0±1), glutamate (+3.0±1), serine (+0.3), asparagine (+0.2), glutamine (+0.2), glycine (0), threonine (−0.4), proline (−0.5±1), alanine (−0.5), histidine (−0.5), cysteine (−1.0), methionine (−1.3), valine (−1.5), leucine (−1.8), isoleucine (−1.8), tyrosine (−2.3), phenylalanine (−2.5), and tryptophan (−3.4).

In making such changes, the substitution of amino acids whose hydrophilicity values are within ±2 is preferred, those that are within ±1 are particularly preferred, and those within ±0.5 are even more particularly preferred.

Variant nucleic acids with silent and conservative changes can be defined and characterized by the degree of homology to the reference nucleic acid. Preferred variant nucleic acids are “substantially homologous” to the reference nucleic acids of the present invention. As recognized by one of skill in the art, such substantially similar nucleic acids can hybridize under stringent conditions with the reference nucleic acids identified by SEQ ID NO herein. These types of substantially homologous nucleic acids are encompassed by this present invention.

Generally, nucleic acid derivatives and variants of the present invention will have at least about 90%, at least about 91%, at least about 92%, at least about 93%, or at least about 94% sequence identity to the reference nucleotide sequence defined herein. Preferably, nucleic acids of the present invention will have at least about 95%, at least about 96%, at least about 97%, or at least about 98% sequence identity to the reference nucleotide sequence defined herein.

Variant nucleic acids can be detected and isolated by standard hybridization procedures. Hybridization to detect or isolate such sequences is generally carried out under “moderately stringent” and preferably under “stringent” conditions. Moderately stringent hybridization conditions and associated moderately stringent and stringent hybridization wash conditions used in the context of nucleic acid hybridization experiments, such as Southern and northern hybridization, are sequence dependent, and are different under different environmental parameters. Longer sequences hybridize specifically at higher temperatures. An extensive guide to the hybridization of nucleic acids is found in Tijssen, Laboratory Techniques in Biochemistry and Molecular biology-Hybridization with Nucleic Acid Probes, page 1, Chapter 2, Overview of principles of hybridization and the strategy of nucleic acid probe assays, Elsevier, N.Y. (1993). See also, J. Sambrook et al., Molecular Cloning: A Laboratory Manual, Cold Spring Harbor Press, NY, pp. 9.31-9.58 (1989); J. Sambrook et al., Molecular Cloning: A Laboratory Manual, Cold Spring Harbor Press, NY (3rd ed. 2001).

The present invention also provides methods for detection and isolation of derivative or variant nucleic acids encoding the proteins provided herein. The methods involve hybridizing at least a portion of a nucleic acid comprising any part of SEQ ID NO: 1, 16, 21, 26, 28, 30, 59, or 61 with respect to FAS-related sequences; and any part of SEQ ID NO: 3, 32, 34, 36, 38, 40, or 42, with respect to phosphopantetheine:protein transferase to a sample nucleic acid, thereby forming a hybridization complex; and detecting the hybridization complex. The presence of the complex correlates with the presence of a derivative or variant nucleic acid that can be further characterized by nucleic acid sequencing, expression of RNA and/or protein and testing to determine whether the derivative or variant retains activity. In general, the portion of a nucleic acid comprising any part of the aforementioned DNAs identified by SEQ ID NO used for hybridization is preferably at least about 15 nucleotides, and hybridization is under hybridization conditions that are sufficiently stringent to permit detection and isolation of substantially homologous nucleic acids; preferably, the hybridization conditions are “moderately stringent”; more preferably the hybridization conditions are “stringent”, as defined herein and in the context of conventional molecular biological techniques well known in the art.

Generally, highly stringent hybridization and wash conditions are selected to be about 5° C. lower than the thermal melting point (Tₘ,) for the specific double-stranded sequence at a defined ionic strength and pH. For example, under “highly stringent conditions” or “highly stringent hybridization conditions” a nucleic acid will hybridize to its complement to a detectably greater degree than to other sequences (e.g., at least 2-fold over background). By controlling the stringency of the hybridization and/or the washing conditions, nucleic acids having 100% complementary can be identified and isolated.

Alternatively, stringency conditions can be adjusted to allow some mismatching in sequences so that lower degrees of similarity are detected (heterologous probing) using, for example, moderately stringent conditions. Appropriate stringency conditions that promote DNA hybridization under moderately stringent conditions are, for example, about 2× sodium chloride/sodium citrate (SSC) at about 65° C., followed by a wash of 2×SSC at 20-25° C., are known to those skilled in the art and can be found in Current Protocols in Molecular Biology, John Wiley & Sons, NY, 6.3.1-6.3.6 (1989). Both temperature and salt may be varied, or either the temperature or the salt concentration may be held constant while the other variable is changed.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide, in which case hybridization temperatures can be decreased. Dextran sulfate and/or Denhardt's solution (50× Denhardt's is 5% Ficoll, 5% polyvinylpyrrolidone, 5% BSA) can also be included in the hybridization reactions.

Exemplary low stringency conditions include hybridization with a buffer solution of 30 to 50% formamide, 5×SSC (20×SSC is 3M NaCl, 0.3 M trisodium citrate), 50 mM sodium phosphate, pH7, 5 mM EDTA, 0.1% SDS (sodium dodecyl sulfate), 5× Denhardt's with 100 Ag/ml denatured salmon sperm DNA at 37° C., and a wash in 1× to 5×SSC (20×SSC=3.0 M NaCl and 0.3 M trisodium citrate), 0.1% SDS at 37° C. Exemplary moderate stringency conditions include hybridization in 40 to 50% formamide, 5×SSC 50 mM sodium phosphate, pH 7, 5 mM EDTA, 0.1% SDS, 5× Denhardt's with 100 μg/ml denatured salmon sperm DNA at 42° C., and a wash in 0.1× to 2×SSC, 0.1% SDS at 42 to 55° C. Exemplary high stringency conditions include hybridization in 50% formamide, 5×SSC, 50 mM sodium phosphate, pH 7.0, 5 mM EDTA, 0.1% SDS, 5× Denhardt's with 100 Ag/ml denatured salmon sperm DNA at 42° C., and a wash in 0.1×SSC, 0.1% SDS at 60 to 65° C.

The degree of complementarity or homology of hybrids obtained during hybridization is typically a function of post-hybridization washes, the critical factors being the ionic strength and temperature of the final wash solution. The type and length of hybridizing nucleic acids also affects whether hybridization will occur and whether any hybrids formed will be stable under a given set of hybridization and wash conditions. For DNA-DNA hybrids, the Tₘ can be approximated from the equation of Meinkoth and Wahl, Anal. Biochem., 138:267-284 (1984);

Tₘ,=81.5° C.+16.6(log M)+0.41(% GC)−0.61(% form)−500/L

where M is the molarity of monovalent cations, % GC is the percentage of guanosine and cytosine nucleotides in the DNA, % form is the percentage of formamide in the hybridization solution, and L is the length of the hybrid in base pairs. The T., is the temperature (under defined ionic strength and pH) at which 50% of a complementary target sequence hybridizes to a perfectly matched probe. Very stringent conditions are selected for hybridization to derivative and variant nucleic acids having a Tₘ equal to the exact complement of a particular probe, less stringent conditions are selected for hybridization to derivative and variant nucleic acids having a Tₘ less than the exact complement of the probe.

In general, Tₘ, is reduced by about 1° C. for each 1% of mismatching. Thus, Tₘ, hybridization, and/or wash conditions can be adjusted to hybridize to sequences of the desired sequence identity. For example, if sequences with greater than about 90% identity are sought, the Tₘ can be decreased by about 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at about 1, about 2, about 3, or about 4° C. lower than the thermal melting point (Tₘ); moderately stringent conditions can utilize a hybridization and/or wash at about 6, about 7, about 8, about 9, or about 10° C. lower than the thermal melting point (Tₘ); low stringency conditions can utilize a hybridization and/or wash at about 11, about 12, about 13, about 14, about 15, or about 20° C. lower than the thermal melting point (Tₘ).

If the desired degree of mismatching results in a Tₘ, of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology-Hybridization with Nucleic Acid Probes, Part 1, Chapter 2, Elsevier, N.Y.; Ausubel et al., eds. (1995) Current Protocols in Molecular Biology, Chapter 2, Greene Publishing and Wiley—Interscience, NY. See Sambrook et al., (1989) Molecular Cloning: A Laboratory Manual, 2d ed., Cold Spring Harbor Laboratory Press, Plainview, N.Y. Using these references and the teachings herein on the relationship between Tₘ, mismatch, and hybridization and wash conditions, those of ordinary skill can generate variants of the present nucleic acids.

In another preferred embodiment of the present invention, the inventive nucleic acids are defined by the percent identity relationship between particular nucleic acids and other members of the class using analytic protocols well known in the art. Such analytic protocols include, but are not limited to: CLUSTAL in the PC/Gene program (available from Intelligenetics, Mountain View, Calif., or in the Omiga program version 2.0 Accelrys Inc., San Diego, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis.). Alignments using these programs can be performed using the default parameters. The CLUSTAL program is well described by Higgins et al., Gene, 73:237-244 (1988); Higgins et al., CABIOS, 5:151153 (1989); Corpet et al., Nucleic Acids Res., 16:10881-90 (1988); Huang et al., CABIOS, 8:155-65 (1992); and Pearson et al., Meth. Mol. Biol., 24:307-331 (1994). The ALIGN program is based on the algorithm of Meyers and Miller, Computer Applic. Biol. Sci., 4:11-17 (1988). The BLAST programs of Altschul et al., J. Mol. Biol., 215:403 (1990), are based on the algorithm of Karlin and Altschul Proc. Natl. Acad. Sci. (U.S.A.), 87:2264-2268 (1990). To obtain gapped alignments for comparison purposes, Gapped BLAST (in BLAST 2.0) can be utilized as described in Altschul et al., Nucleic Acids Res., 25:3389 (1997). Alternatively, PSI-BLAST (in BLAST 2.0) can be used to perform an iterated search that detects distant relationships between molecules. See, Altschul et al., supra. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (see, Henikoff & Henikoff, Proc. Natl. Acad. Sci. (U.S.A.), 89:10915, 1989). Alignment may also be performed manually by inspection.

For purposes of the present invention, comparison of nucleotide sequences for determination of percent sequence identity to the nucleic acid sequences disclosed herein is preferably made using the BLASTN program (version 1.4.7 or later) with its default parameters or any equivalent program. By “equivalent program” is intended any sequence comparison program that, for any 2 sequences in question, generates an alignment having identical nucleotide or amino acid residue matches and an identical percent sequence identity when compared to the corresponding alignment generated by the preferred program.

### Isolation of Nucleic Acids Encoding Multifunctional Fatty Acid Synthases:

Nucleic acids encoding a multifunctional fatty acid synthase can be identified and isolated by standard methods, as described by Sambrook et al., Molecular Cloning: A Laboratory Manual, Cold Spring Harbor, N.Y. (1989). For example, a DNA sequence encoding a type I multifunctional fatty acid synthase can be identified by screening of a DNA or cDNA library generated from nucleic acid derived from a particular cell type, cell line, primary cells, or tissue. Examples of libraries useful for identifying and isolating a multifunctional fatty acid synthase include libraries made from the genomic DNA or cDNA of any organism encoding a type I fatty acid synthase, preferably a bacteria or non-plant eukaryote.

Screening for DNA fragments that encode a multifunctional fatty acid synthase can be accomplished by screening colonies or plaques from a genomic or cDNA library for hybridization to a probe of an available multifunctional fatty acid synthase from other organisms or by screening colonies or plaques from a cDNA expression library for binding to antibodies that specifically recognize a multifunctional fatty acid synthase. DNA fragments that hybridize to multifunctional fatty acid synthase probes from other organisms and/or colonies or plaques carrying DNA fragments that are immunoreactive with antibodies to multifunctional fatty acid synthase can be subcloned into a vector and sequenced and/or used as probes to identify other cDNA or genomic sequences encoding all or a portion of the desired multifunctional fatty acid synthase gene. Probes for isolation of multifunctional fatty acid synthase genes can also include DNA fragments of type II fatty acid synthase genes or antibodies to the type II proteins, as noted herein above.

A cDNA library can be prepared, for example, by random oligo priming or oligo dT priming. Plaques containing DNA fragments can be screened with probes or antibodies specific for multifunctional fatty acid synthase. DNA fragments encoding a portion of a multifunctional fatty acid synthase gene can be subcloned and sequenced and used as probes to identify a genomic multifunctional fatty acid synthase gene. DNA fragments encoding a portion of a multifunctional fatty acid synthase can be verified by determining sequence homology with other known multifunctional fatty acid synthase genes or by hybridization to multifunctional fatty acid synthase-specific messenger RNA. Once cDNA fragments encoding portions of the 5′, middle and 3′ ends of a multifunctional fatty acid synthase are obtained, they can be used as probes to identify and clone a complete genomic copy of the multifunctional fatty acid synthase gene from a genomic library.

Portions of the genomic copy or copies of an multifunctional fatty acid synthase gene can be sequenced and the 5′ end of the gene identified by standard methods, including either DNA sequence homology to other multifunctional fatty acid synthase genes or by RNAase protection analysis, as described by Sambrook et al., Molecular Cloning: A Laboratory Manual, Cold Spring Harbor Press, Cold Spring Harbor, N.Y. (1989). The 3′ and 5′ ends of the target gene can also be located by computer searches of genomic sequence databases using known fatty acid synthase coding regions. Once portions of the 5′ end of the gene are identified, complete copies of the multifunctional fatty acid synthase gene can be obtained by standard methods, including cloning or polymerase chain reaction (PCR) synthesis using oligonucleotide primers complementary to the DNA sequence at the 5′ end of the gene. The presence of an isolated full-length copy of the multifunctional fatty acid synthase gene can be verified by hybridization, partial sequence analysis, or by expression of the multifunctional fatty acid synthase enzyme.

Phosphopantetheine:Protein Transferases: During the process of fatty acid synthesis, the growing acyl chain is preferably covalently linked by a thioester bond to the cysteamine thiol of a phosphopantetheinyl (P-Pan) moiety, which is preferably attached at the other end to a specific serine residue of acyl carrier protein (ACP), in the case of type II FAS systems, or the ACP-domain of a type I FAS. This P-Pan moiety acts as a “swinging arm,” carrying the growing acyl chain between the active sites of the different enzymes or domains of the FAS complex. Accordingly, the transgenic mfFAS used in the context of the present invention is preferably phosphopantetheinylated, which phosphopantetheinylation is accomplished by a co-transformed gene that encodes a suitable PPTase or by a host PPTase that has sufficient substrate range of activity for the purpose of modifying the transgenic mfFAS.

The enzymatic post-translational attachment of the P-Pan group to an ACP protein or domain is carried out by a phosphopantetheinyl transferase (PPTase). Any suitable PPTase can be used in the context of the present invention, the suitability of which is determined by the ability of the PPTase to phosphopantetheinylate a mfFAS used herein. For example, the Brevibacterium ammoniagenes FasA protein (SEQ ID NO: 2) can be suitably combined with the PPTase from the same species, which is identified herein as SEQ ID NO: 4. In another embodiment of the present invention, the gene encoding the mfFAS includes its own PPTase activity, such as the mfFAS derived from yeast (e.g., SEQ ID NOs: 15-19), and thus the transgenic mfFAS is suitably modified to be active upon expression in the host. Particularly preferred PPTases have broad specificity, such as, for example, those referred to as being of the sfp-type, as further discussed hereinbelow. More preferred, the mfFAS employed in the context of the present invention is pantethenylated by an enzyme having PPTase activity that is native to the host Brassica plant into which the mfFAS transgene has been inserted.

A PPTase from Bacillus subtillis, the sfp gene product, has a remarkably broad range of substrate specificity, being able to phosphopantetheinylate non-native substrates both in vitro (Lambalot et al., Chem. Biol., 3:923-936, 1996) and in vivo (Mootz et al., J. Biol. Chem., 276:37389-37298, 2001); see FIG. 23 for recital of the sequences of the sfp gene and its product. Mootz and co-workers have shown that the sfp gene product not only complements heterologous PPTases, such as E. coli ACPS, but it in vivo phosphopantetheinylates all the different acceptor domains in natural host cells (e.g., Bacillus subtillis) that include ACP and PCP (peptide carrier protein) of type I polyketide synthases (PKS) and non-ribosomal peptide synthetases (NRPS) involved in secondary metabolism as well as the type II ACP protein required for fatty acid synthesis (primary metabolism). Indeed, this broad range of specificity appears to be a general feature of many sfp-type PPTases. Streptomyces verticullus svp PPTase (see FIG. 26), another sfp-type enzyme, was also found to be able to phosphopantetheinylate a broad range of substrates, including type I and II ACP and PCP domains from various Streptomyces species (Sanchez et al., Chem. Biol., 8:725-728, 2001). Other useful sfp-type PPTases include those found in Brevibacillus brevis (SEQ ID NO: 35), and Escherichia coli (SEQ ID NO: 36), which are listed here without any intention to limit the sfp-type PPTases that are usefully employed in the context of the present invention. Preferably, the Bacillus subtilis PPTase, that is the gene that encodes it, is used.

In the case of the multifunctional FasA and FasB proteins from Brevibacterium ammoniagenes, Stuible and co-workers found that the E. coli ACPS was unable to phosphopantetheinylate these type I FAS proteins either in vivo when the genes were introduced into E. coli or in vitro when mixed with the proteins. The B. ammoniagenes PPT1 protein was required to phosphopantetheinylate both of these type I FAS proteins (Stuible et al., Eur. J. Biochem., 248:481-487, 1997).

A preferred embodiment of the present invention relates to the use of an mfFAS that can be phosphopantetheinylated by a PPTase that is innate to a Brassica plant. An alternative preferred embodiment relates to the use of a PPTase specific for the introduced multifunctional FAS that is inserted in a Brassica plant, such as pptl in the case of the B. ammoniagenes fasA and fasB genes, which specific PPTase could be co-expressed in order to engineer functional multifunctional FAS expression in Brassica plants. As a further embodiment of the present invention, a PPTase of broad specificity, such as a sfp-type PPTase, may be co-expressed with a type II FAS gene in order to engineer functional multifunctional FAS expression in Brassica plants.

Expression Vectors and Cassettes: The expression vectors and cassettes of the present invention include nucleic acids encoding multifunctional fatty acid synthases. When inclusion of a heterologous phosphopantetheine protein transferase enzyme (PPTase) is desired, such expression vectors and cassettes can also include a nucleic acid encoding a PPTase that can post-translationally activate the multifunctional fatty acid synthase polypeptide. Alternatively, a separate expression vector or cassette can encode a phosphopantetheine protein transferase enzyme. One such PPTase is encoded by the B. ammoniagenes pptl gene. Other sources of PPTase having broad spectrum activity include: Bacillus subtilis, Brevibacillus brevis, Escherichia coli, Streptomyces verticullus, and Saccharomyces cerevisiae.

A transgene comprising a multifunctional fatty acid synthase can be subcloned into an expression vector or cassette, and fatty acid synthase expression can be detected and/or quantified. This method of screening is useful to identify transgenes providing for an expression of a multifunctional fatty acid synthase, and expression of a multifunctional fatty acid synthase in a transformed Brassica plant cell.

Plasmid vectors that provide for easy selection, amplification, and transformation of the transgene in prokaryotic and eukaryotic cells include, for example, pUC-derived vectors, pSK-derived vectors, pGEM-derived vectors, pSP-derived vectors, pBS-derived vectors, pFastBac (Invitrogen) for baculovirus expression and pYES2 (Invitrogen) for yeast expression. Additional elements may be present in such vectors, including origins of replication to provide for autonomous replication of the vector, selectable marker genes, preferably encoding antibiotic or herbicide resistance, unique multiple cloning sites providing for multiple sites to insert DNA sequences or genes encoded in the transgene, and sequences that enhance transformation of prokaryotic and eukaryotic cells. One vector that is useful for expression in both plant and prokaryotic cells is the binary Ti plasmid (as disclosed in Schilperoot et al., U.S. Pat. No. 4,940,838), as exemplified by vector pGA582. This binary Ti plasmid vector has been previously characterized by An, Methods in Enzymology, 153:292 (1987). This binary Ti vector can be replicated in prokaryotic bacteria, such as E. coli and Agrobacterium. The Agrobacterium plasmid vectors can also be used to transfer the transgene to Brassica plant cells. The binary Ti vectors preferably include the nopaline T DNA right and left borders to provide for efficient Brassica plant cell transformation, a selectable marker gene, unique multiple cloning sites in the T border regions, the colE1 replication of origin and a wide host range replicon. The binary Ti vectors carrying a transgene of the present invention can be used to transform both prokaryotic and eukaryotic cells, but is preferably used to transform plant cells. See, for example, Glassman et al., U.S. Pat. No. 5,258,300.

In general, the expression vectors and cassettes of the present invention contain at least a promoter capable of expressing RNA in a Brassica plant cell and a terminator, in addition to a nucleic acid encoding a multifunctional fatty acid synthase. Other elements may also be present in the expression cassettes of the present invention. For example, expression cassettes can also contain enhancers, introns, untranslated leader sequences, cloning sites, matrix attachment regions for silencing the effects of chromosomal control elements, and other elements known to one of skill in the art.

Nucleic acids encoding fatty acid synthases are operably linked to regulatory elements, such as a promoter, termination signals, and the like. Operably linking a nucleic acid under the regulatory control of a promoter or a regulatory element means positioning the nucleic acid such that the expression of the nucleic acid is controlled by these sequences. In general, promoters are found positioned 5′ (upstream) to the nucleic acid that they control. Thus, in the construction of heterologous promoter/nucleic acid combinations, the promoter is preferably positioned upstream to the nucleic acid and at a distance from the transcription start site of the nucleic acid that the distance between the promoter and the transcription start site approximates the distance observed in the natural setting. As is known in the art, some variation in this distance can be tolerated without loss of promoter function. Similarly, the preferred positioning of a regulatory element with respect to a heterologous nucleic acid placed under its control is the natural position of the regulatory element relative to the structural gene it naturally regulates. Again, as is known in the art, some variation in this distance can be accommodated.

Expression cassettes have promoters that can regulate gene expression. Promoter regions are typically found in the flanking DNA sequence upstream from coding regions in both prokaryotic and eukaryotic cells. A promoter sequence provides for regulation of transcription of the downstream gene sequence and typically includes from about 50 to about 2,000 nucleotide base pairs. Promoter sequences also contain regulatory sequences, such as enhancer sequences that can influence the level of gene expression. Some isolated promoter sequences can provide for gene expression of heterologous genes, that is, a gene different from the native or homologous gene. Promoter sequences are also known to be strong or weak or inducible. A strong promoter provides for a high level of gene expression, whereas a weak promoter provides for a very low level of gene expression. An inducible promoter is a promoter that provides for turning on and off of gene expression in response to an exogenously added agent or to an environmental or developmental stimulus. Promoters can also provide for tissue specific or developmental regulation. An isolated promoter sequence that is a strong promoter for heterologous genes is advantageous because it provides for a sufficient level of gene expression to allow for easy detection and selection of transformed cells and provides for a high level of gene expression when desired. Transcription initiation regions that are preferentially expressed in seed tissue, and that are undetectable in other Brassica plant parts, are considered desirable for seed oil modifications in order to minimize any disruptive or adverse effects of the gene product.

Promoters of the present invention will generally include, but are not limited to, promoters that function in bacteria, bacteriophage, plastids, or plant cells. Useful promoters include the globulin promoter (see, for example, Belanger and Kriz, Genet., 129:863-872, 1991), gamma zein Z27 promoter (see, for example, U.S. Ser. No. 08/763,705; also Lopes et al., Mol Gen Genet., 247:603-613, 1995), L3 oleosin promoter (U.S. Pat. No. 6,433,252), USP promoter and 7Sa promoter (U.S. Ser. No. 10/235,618), 7Sa′ promoter (see, for example, Beachy et al., EMBO J., 4:3047, 1985; Schuler et al., Nucleic Acid Res., 10(24):8225-8244, 1982), CaMV 35S promoter (Odell et al., Nature, 313:810, 1985), the CaMV 19S (Lawton et al., Plant Mol. Biol., 9:31F, 1987), nos (Ebert et al., Proc. Natl. Acad. Sci. (U.S.A.), 84:5745, 1987), Adh (Walker et al., Proc. Natl. Acad. Sci. (U.S.A.), 84:6624, 1987), sucrose synthase (Yang et al., Proc. Natl. Acad. Sci. (U.S.A.), 87:4144, 1990), tubulin, actin (Wang et al., Mol. Cell. Biol., 12:3399, 1992), cab (Sullivan et al., Mol. Gen. Genet., 215:431, 1989), PEPCase promoter (Hudspeth et al., Plant Mol. Biol., 12:579, 1989), or those associated with the R gene complex (Chandler et al., The Plant Cell, 1:1175, 1989). Other useful promoters include the Figwort Mosaic Virus (FMV) promoter (Richins et al., Nucleic Acids Res., 20:8451, 1987), arcelin, tomato E8, patatin, ubiquitin, mannopine synthase (mas), soybean seed protein glycinin (Gly), soybean vegetative storage protein (vsp), bacteriophage SP6, T3, and T7 promoters.

Indeed, in a preferred embodiment, the promoter used is a seed-specific promoter. Examples of seed regulated genes and transcriptional regions are disclosed in U.S. Pat. Nos. 5,420,034; 5,608,152; and 5,530,194. Examples of such promoters include the 5′ regulatory regions from such genes as napin (Kridl et al., Seed Sci. Res., 1:209-219, 1991), phaseolin (Bustos et al., Plant Cell, 1(9):839-853, 1989), soybean trypsin inhibitor (Riggs et al., Plant Cell, 1(6):609-621, 1989), ACP (Baerson et al., Plant Mol. Biol., 22(2):255-267, 1993), stearoyl-ACP desaturase (Slocombe et al., Plant Physiol., 104(4):167-176, 1994), soybean a′ subunit of ii-conglycinin (Chen et al., Proc. Natl. Acad. Sci., 83:8560-8564, 1986), Lesquerella hydroxylase promoter (described in Broun et al., Plant Journal, 12(2):201-210, 1998; U.S. Pat. No. 5,965,793), delta 12 desaturase and oleosin (Hong et al., Plant Mol. Biol., 34(3):549-555, 1997). Further examples include the promoter for I3-conglycinin (Chen et al., Dev. Genet., 10:112-122, 1989), the GL2 promoter (Szymanski et al., Development, 125:1161-1171, 1998), the tt2 promoter (Nesi et al., The Plant Cell, 13:2099-114, 2001), the LDOX promoter (Pelletier et al., Plant Physiology, 113:1437-1445, 1997), the CPC promoter (Wada et al., Science, 277:1113-1116, 1997).

Plastid promoters can also be used. Most plastid genes contain a promoter for the multi-subunit plastid-encoded RNA polymerase (PEP) as well as the single-subunit nuclear-encoded RNA polymerase. A consensus sequence for the nuclear-encoded polymerase (NEP) promoters and listing of specific promoter sequences for several native plastid genes can be found in Hajdukiewicz et al., EMBO J., 16:4041-4048 (1997), which is hereby in its entirety incorporated by reference.

Examples of plastid promoters that can be used include the Zea mays plastid RRN (ZMRRN) promoter. The ZMRRN promoter can drive expression of a gene when the Arabidopsis thaliana plastid RNA polymerase is present. Similar promoters that can be used in the present invention are the Glycine max plastid RRN (SOYRRN) and the Nicotiana tabacum plastid RRN (NTRRN) promoters. All three promoters can be recognized by the Arabidopsis plastid RNA polymerase. The general features of RRN promoters are described by Hajdukiewicz et al., supra, and U.S. Pat. No. 6,218,145.

Moreover, transcription enhancers or duplications of enhancers can be used to increase expression from a particular promoter. Examples of such enhancers include, but are not limited to, elements from the CaMV 35S promoter and octopine synthase genes (Last et al., U.S. Pat. No. 5,290,924). As the DNA sequence between the transcription initiation site and the start of the coding sequence, i.e., the untranslated leader sequence, can influence gene expression, one may also wish to employ a particular leader sequence. Any leader sequence available to one of skill in the art may be employed. Preferred leader sequences direct optimum levels of expression of the attached gene, for example, by increasing or maintaining mRNA stability and/or by preventing inappropriate initiation of translation (Joshi, Nucl. Acid Res., 15:6643, 1987). The choice of such sequences is at the discretion of those of skill in the art. Sequences that are derived from genes that are highly expressed in Brassica in particular are contemplated.

An inducible promoter can be turned on or off by an exogenously added agent so that expression of an operably linked nucleic acid is also turned on or off. For example, a bacterial promoter, such as the Ptac, promoter can be induced to varying levels of gene expression depending on the level of isothiopropylgalactoside added to the transformed bacterial cells. It may also be preferable to combine the nucleic acid encoding the polypeptide of interest with a promoter that provides tissue specific expression or developmentally regulated gene expression in plants.

Expression cassettes of the present invention will also include a sequence near the 3′ end of the cassette that acts as a signal to terminate transcription from a heterologous nucleic acid and that directs polyadenylation of the resultant mRNA. Some 3′ elements that can act as termination signals include those from the nopaline synthase gene of Agrobacterium tumefaciens (Bevan et al., Nucl. Acid Res., 11:369, 1983), a napin 3′ untranslated region (Kridl et al., Seed Sci Res., 1:209-219, 1991), a globulin 3′ untranslated region (Belanger and Kriz, Genetics, 129:863-872, 1991), or one from a zein gene, such as Z27 (Lopes et al., Mol. Gen. Genet., 247:603-613, 1995). Other 3′ elements known by one of skill in the art also can be used in the vectors of the present invention.

Regulatory elements, such as Adh intron 1 (Callis et al., Genes Develop., 1:1183, 1987), a rice actin intron (McElroy et al., Mol. Gen. Genet., 231(1):150-160, 1991), sucrose synthase intron (Vasil et al., Plant Physiol., 91:5175, 1989), the maize HSP70 intron (Rochester et al., EMBO J., 5:451-458, 1986), or TMV omega element (Gallie et al., The Plant Cell, 1:301, 1989) may further be included where desired. These 3′ nontranslated regulatory sequences can be obtained as described in An, Methods in Enzymology, 153:292 (1987) or are already present in plasmids available from commercial sources, such as Clontech, Palo Alto, Calif. The 3′ nontranslated regulatory sequences can be operably linked to the 3′ terminus of any heterologous nucleic acid to be expressed by the expression cassettes contained within the present vectors. Other such regulatory elements useful in the practice of the present invention are known by one of skill in the art and can also be placed in the vectors of the present invention.

The vectors of the present invention, as well as the coding regions claimed herein, can be optimized for expression in Brassica plants by having one or more codons replaced by other codons encoding the same amino acids so that the polypeptide is optimally translated by the translation machinery of the Brassica plant species in which the vector is used.

Selectable Markers: Selectable marker genes or reporter genes are also useful in the present invention. Such genes can impart a distinct phenotype to cells expressing the marker gene and thus allow such transformed cells to be distinguished from cells that do not have the marker. Selectable marker genes confer a trait that one can ‘select’ for by chemical means, i.e., through the use of a selective agent (e.g., a herbicide, antibiotic, or the like). Reporter genes or screenable genes, confer a trait that one can identify through observation or testing, i.e., by ‘screening’ (e.g., the R-locus trait). Of course, many examples of suitable marker genes are known to the art and can be employed in the practice of the present invention.

Possible selectable markers for use in connection with the present invention include, but are not limited to, a neo gene (Potrykus et al., Mol. Gen. Genet., 199:183, 1985) which codes for kanamycin resistance and can be selected for by applying kanamycin, a kanamycin analog such as geneticin (Sigma Chemical Company, St. Louis, Mo.), and the like; a bar gene that codes for bialaphos resistance; a gene that encodes an altered EPSP synthase protein (Hinchee et al., Biotech., 6:915, 1988) thus conferring glyphosate resistance; a nitrilase gene, such as bxn from Klebsiella ozaenae, which confers resistance to bromoxynil (Stalker et al., Science, 242:419, 1988); a mutant acetolactate synthase gene (ALS) that confers resistance to imidazolinone, sulfonylurea, or other ALS-inhibiting chemicals (EP 154 204A1, 1985); a methotrexate-resistant DHFR gene (Thillet et al., J. Biol. Chem., 263:12500, 1988); a dalapon dehalogenase gene that confers resistance to the herbicide dalapon. Where a mutant EPSP synthase gene is employed, additional benefit may be realized through the incorporation of a suitable plastid transit peptide (CTP).

An illustrative embodiment of a selectable marker gene capable of being used in systems to select transformants is the genes that encode the enzyme phosphinothricin acetyltransferase, such as the bar gene from Streptomyces hygroscopicus or the pat gene from Streptomyces viridochromogenes (U.S. Pat. No. 5,550,318, which is incorporated by reference herein). The enzyme phosphinothricin acetyl transferase (PAT) inactivates the active ingredient in the herbicide bialaphos, phosphinothricin that inhibits glutamine synthetase, (Murakami et al., Mol. Gen. Genet., 205:42, 1986; Twell et al., Plant Physiol., 91:1270, 1989) causing rapid accumulation of ammonia and cell death.

Screenable markers that may be employed include, but are not limited to, a β-glucuronidase or uidA gene (GUS), which encodes an enzyme for which various chromogenic substrates are known; an R-locus gene, which encodes a product that regulates the production of anthocyanin pigments (red color) in plant tissues (Dellaporta et al., Chromosome Structure and Function, 263-282, 1988); a β-lactamase gene (Sutcliffe, Proc. Natl. Acad. Sci. (U.S.A.), 75:3737, 1978), which encodes an enzyme for which various chromogenic substrates are known (e.g., PADAC, a chromogenic cephalosporin); a xylE gene (Zukowsky et al., Proc. Natl. Acad. Sci. (U.S.A.), 80:1101, 1983) that encodes a catechol dioxygenase that can convert chromogenic catechols; an a-amylase gene (Ikuta et al., Biotech., 8:241, 1990); a tyrosinase gene (Katz et al., J. Gen. Microbiol., 129:2703, 1983) that encodes an enzyme capable of oxidizing tyrosine to DOPA and dopaquinone, which in turn condenses to form the easily detectable compound melanin; a P-galactosidase gene, which encodes an enzyme for which there are chromogenic substrates; a luciferase (lux) gene (Ow et al., Science, 234:856, 1986), which allows for bioluminescence detection; or an aequorin gene (Prasher et al., Biochem. Biophys. Res. Comm., 126:1259, 1985), which may be employed in calcium-sensitive bioluminescence detection, or a green fluorescent protein gene (Niedz et al., Plant Cell Reports, 14:403, 1995). The presence of the lux gene in transformed cells may be detected using, for example, X-ray film, scintillation counting, fluorescent spectrophotometry, low-light video cameras, photon-counting cameras, or multiwell luminometry. It is also envisioned that this system may be developed for populational screening for bioluminescence, such as on tissue culture plates, or even for whole plant screening.

Transit Peptides: Additionally, transgenes may be constructed and employed to provide targeting of the gene product to an intracellular compartment within plant cells or in directing a protein to the extracellular environment. This will generally be achieved by joining a DNA sequence encoding a transit or signal peptide sequence to the coding sequence of a particular gene. The resultant transit or signal peptide will transport the protein to a particular intracellular, or extracellular destination, respectively, and may then be posttranslationally removed. Transit or signal peptides act by facilitating the transport of proteins through intracellular membranes, e.g., vacuole, vesicle, plastid, and mitochondrial membranes, whereas signal peptides direct proteins through the extracellular membrane. By facilitating transport of the protein into compartments inside or outside the cell, these sequences may increase the accumulation of gene product.

An example of such a use concerns the direction of a fatty acid synthase to a particular organelle, such as to a plastid rather than to the cytoplasm. This is exemplified by the use of the Arabidopsis SSU1A transit peptide that confers plastid-specific targeting of proteins. Alternatively, the transgene can comprise a plastid transit peptide-encoding DNA sequence or a DNA sequence encoding the rbcS (RuBISCO) transit peptide operably linked between a promoter and the DNA sequence encoding a fatty acid synthase (for a review of plastid targeting peptides, see Heijne et al., Eur. J. Biochem., 180:535, 1989; Keegstra et al., Ann. Rev. Plant Physiol. Plant Mol. Biol., 40:471, 1989). If the transgene is to be introduced into a plant cell, the transgene can also contain plant transcriptional termination and polyadenylation signals and translational signals linked to the 3′ terminus of a plant fatty acid synthase gene.

A heterologous plastid transit peptide can be linked to a multifunctional fatty acid synthase gene. A plastid transit peptide is typically 40 to 70 amino acids in length and functions post-translationally to direct a protein to the plastid. The transit peptide is cleaved either during or just after import into the plastid to yield the mature protein.

Heterologous plastid transit peptide encoding sequences can be obtained from a variety of plant nuclear genes, so long as the products of the genes are expressed as preproteins comprising an amino terminal transit peptide and transported into plastid. Examples of plant gene products known to include such transit peptide sequences include, but are not limited to, the small subunit of ribulose biphosphate carboxylase, chlorophyll a/b binding protein, plastid ribosomal proteins encoded by nuclear genes, certain heat shock proteins, amino acid biosynthetic enzymes, such as acetolactate acid synthase, 3-enolpyruvylphosphoshikimate synthase, dihydrodipicolinate synthase, fatty acid synthase, and the like. In some instances, a plastid transport protein already may be encoded in the fatty acid synthase gene of interest, in which case there may be no need to add such plastid transit sequences. Alternatively, the DNA fragment coding for the transit peptide may be chemically synthesized either wholly or in part from the known sequences of transit peptides such as those listed above.

Regardless of the source of the DNA fragment coding for the transit peptide, it should include a translation initiation codon, for example, an ATG codon, and be expressed as an amino acid sequence that is recognized by and will function properly in plastids of the host plant. Attention should also be given to the amino acid sequence at the junction between the transit peptide and the fatty acid synthase enzyme, where it is cleaved to yield the mature enzyme. Certain conserved amino acid sequences have been identified and may serve as a guideline. Precise fusion of the transit peptide coding sequence with the fatty acid synthase coding sequence may require manipulation of one or both DNA sequences to introduce, for example, a convenient restriction site. This may be accomplished by methods including site-directed mutagenesis, insertion of chemically synthesized oligonucleotide linkers, and the like.

Precise fusion of the nucleic acids encoding the plastid transport protein may not be necessary so long as the coding sequence of the plastid transport protein is in-frame with that of the fatty acid synthase. For example, additional peptidyl or amino acids can often be included without adversely affecting the expression or localization of the protein of interest.

Once obtained, and when desired, the plastid transit peptide sequence can be appropriately linked to the promoter and a fatty acid synthase coding region in a transgene using standard methods. A plasmid containing a promoter functional in plant cells and having multiple cloning sites downstream can be constructed or obtained from commercial sources.

The plastid transit peptide sequence can be inserted downstream from the promoter using restriction enzymes. A fatty acid synthase coding region can then be translationally fused or inserted immediately downstream from and in frame with the 3′ terminus of the plastid transit peptide sequence. Hence, the plastid transit peptide is preferably linked to the amino terminus of the fatty acid synthase. Once formed, the transgene can be subcloned into other plasmids or vectors.

In addition to nuclear plant transformation, the present invention also extends to direct transformation of the plastid genome of Brassica plants. Hence, targeting of the gene product to an intracellular compartment within plant cells may also be achieved by direct delivery of a gene to the intracellular compartment. In some embodiments, direct transformation of plastid genome may provide additional benefits over nuclear transformation. For example, direct plastid transformation of fatty acid synthase eliminates the requirement for a plastid targeting peptide and post-translational transport and processing of the pre-protein derived from the corresponding nuclear transformants. Plastid transformation of plants has been described by Maliga, Current Opinion in Plant Biology, 5:164-172 (2002); Heifetz, Biochimie, 82:655-666 (2000); Bock, J. Mol. Biol., 312:425-438 (2001); and Daniell et al., Trends in Plant Science, 7:84-91 (2002), and references cited therein.

After constructing a transgene containing a multifunctional fatty acid synthase, the expression vector or cassette can then be introduced into a Brassica plant cell. Depending on the type of plant cell, the level of gene expression, and the activity of the enzyme encoded by the gene, introduction of DNA encoding a multifunctional fatty acid synthase into the plant cell can lead to increased oil content in Brassica plant tissues.

Plant Transformation: There are many methods for introducing transforming nucleic acid molecules into plant cells. Suitable methods are believed to include virtually any method by which nucleic acid molecules may be introduced into a cell, such as by Agrobacterium infection or direct delivery of nucleic acid molecules, such as, for example, by PEG-mediated transformation, by electroporation or by acceleration of DNA coated particles, and the like. (Potrykus, Ann. Rev. Plant Physiol. Plant Mol. Biol., 42:205-225, 1991; Vasil, Plant Mol. Biol., 25:925-937, 1994). For example, electroporation has been used to transform maize protoplasts (Fromm et al., Nature, 312:791-793, 1986).

Other vector systems suitable for introducing transforming DNA into a host plant cell include but are not limited to binary artificial chromosome (BIBAC) vectors (Hamilton et al., Gene, 200:107-116, 1997); and transfection with RNA viral vectors (Della-Cioppa et al., Ann. N.Y. Acad. Sci., (1996), 792 (Engineering Plants for Commercial Products and Applications, 57-61)). Additional vector systems also include plant selectable YAC vectors, such as those described in Mullen et al., Molecular Breeding, 4:449-457 (1988).

Technology for introduction of DNA into cells is well known by one of skill in the art. Four general methods for delivering a gene into cells have been described: (1) chemical methods (Graham and van der Eb, Virology, 54:536-539, 1973); (2) physical methods, such as microinjection (Capecchi, Cell, 22:479-488, 1980), electroporation (Wong and Neumann, Biochem. Biophys. Res. Commun., 107:584-587, 1982; Fromm et al., Proc. Natl. Acad. Sci. (U.S.A.), 82:5824-5828, 1985; U.S. Pat. No. 5,384,253); the gene gun (Johnston and Tang, Methods Cell Biol., 43:353-365, 1994); and vacuum infiltration (Bechtold et al., C.R. Acad. Sci. Paris, Life Sci., 316:1194-1199, 1993); (3) viral vectors (Clapp, Clin. Perinatol., 20:155-168, 1993; Lu et al., J. Exp. Med., 178:2089-2096, 1993; Eglitis and Anderson, Biotechniques, 6:608-614, 1988); and (4) receptor-mediated mechanisms (Curie et al., Hum. Gen. Ther., 3:147-154, 1992; Wagner et al., Proc. Natl. Acad. Sci. (U.S.A.), 89:6099-6103, 1992).

Acceleration methods that may be used include, for example, microprojectile bombardment and the like. One example of a method for delivering transforming nucleic acid molecules into plant cells is microprojectile bombardment. This method has been reviewed by Yang and Christou (eds.), Particle Bombardment Technology for Gene Transfer, Oxford Press, Oxford, England (1994). Non-biological particles (microprojectiles) may be coated with nucleic acids and delivered into cells by a propelling force. Exemplary particles include those comprised of tungsten, gold, platinum, and the like.

A particular advantage of microprojectile bombardment, in addition to it being an effective means of reproducibly transforming monocots, is that neither the isolation of protoplasts (Christou et al., Plant Physiol., 87:671-674, 1988) nor the susceptibility to Agrobacterium infection is required. An illustrative embodiment of a method for delivering DNA into maize cells by acceleration is a biolistics a-particle delivery system, which can be used to propel particles coated with DNA through a screen, such as a stainless steel or NYTEX screen, onto a filter surface covered with maize cells cultured in suspension. Gordon-Kamm et al., describes the basic procedure for coating tungsten particles with DNA (Gordon-Kamm et al., Plant Cell, 2:603-618, 1990). The screen disperses the tungsten nucleic acid particles so that they are not delivered to the recipient cells in large aggregates. A particle delivery system suitable for use with the present invention is the helium acceleration PDS-1000/He gun, which is available from Bio-Rad Laboratories (Bio-Rad, Hercules, Calif.) (also, see, Sanford et al., Technique, 3:3-16, 1991).

For the bombardment, cells in suspension may be concentrated on filters. Filters containing the cells to be bombarded are positioned at an appropriate distance below the microprojectile stopping plate. If desired, one or more screens are also positioned between the gun and the cells to be bombarded.

Alternatively, immature embryos or other target cells may be arranged on solid culture medium. The cells to be bombarded are positioned at an appropriate distance below the microprojectile stopping plate. If desired, one or more screens are also positioned between the acceleration device and the cells to be bombarded. Through the use of techniques set forth herein one may obtain 1000 or more loci of cells transiently expressing a marker gene. The number of cells in a focus that express the exogenous gene product 48 hours post-bombardment often ranges from 1 to 10, and average 1 to 3.

In bombardment transformation, one may optimize the pre-bombardment culturing conditions and the bombardment parameters to yield the maximum numbers of stable transformants. Both the physical and biological parameters for bombardment are important in this technology. Physical factors are those that involve manipulating the DNA/microprojectile precipitate or those that affect the flight and velocity of the microprojectiles. Biological factors include all steps involved in manipulation of cells before and immediately after bombardment, the osmotic adjustment of target cells to help alleviate the trauma associated with bombardment and, also, the nature of the transforming DNA, such as linearized DNA or intact supercoiled plasmids. It is believed that pre-bombardment manipulations are especially important for successful transformation of immature embryos.

In another alternative embodiment, plastids can be stably transformed. Methods disclosed for plastid transformation in higher plants include the particle gun delivery of DNA containing a selectable marker and targeting of the DNA to the plastid genome through homologous recombination (Svab et al., Proc. Nat'l. Acad. Sci. (U.S.A.), 87:8526-8530, 1990; Svab and Maliga, Proc. Natl. Acad. Sci. (U.S.A.), 90:913-917, 1993; Staub and Maliga, EMBO J., 12:601-606, 1993; U.S. Pat. Nos. 5,451,513 and 5,545,818).

Accordingly, it is contemplated that one may wish to adjust various aspects of the bombardment parameters in small scale studies to fully optimize the conditions. One may particularly wish to adjust physical parameters such as gap distance, flight distance, tissue distance, and helium pressure. One may also minimize the trauma reduction factors by modifying conditions that influence the physiological state of the recipient cells and which may therefore influence transformation and integration efficiencies. For example, the osmotic state, tissue hydration, and the subculture stage or cell cycle of the recipient cells may be adjusted for optimum transformation. The execution of other routine adjustments will be known by one of skill in the art in light of the present invention.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example the methods described by Fraley et al., Bio/technology, 3:629-635 (1985) and Rogers et al., Methods Enzymol., 153:253-277 (1987). Further, the integration of the Ti-DNA is a relatively precise process resulting in few rearrangements. The region of DNA to be transferred is defined by the border sequences and intervening DNA is usually inserted into the plant genome as described (Spielmann et al., Mol. Gen. Genet., 205:34, 1986).

Modern Agrobacterium transformation vectors are capable of replication in E. coli as well as Agrobacterium, allowing for convenient manipulations as described (Klee et al., In: Plant DNA Infectious Agents, Hohn and Schell (eds.), Springer-Verlag, NY, pp. 179-203, 1985). Moreover, technological advances in vectors for Agrobacterium-mediated gene transfer have improved the arrangement of genes and restriction sites in the vectors to facilitate construction of vectors capable of expressing various polypeptide coding genes. The vectors described have convenient multi-linker regions flanked by a promoter and a polyadenylation site for direct expression of inserted polypeptide coding genes and are suitable for present purposes (Rogers et al., Methods Enzymol., 153:253-277, 1987). In addition, Agrobacterium containing both armed and disarmed Ti genes can be used for the transformations. In those plant strains where Agrobacterium-mediated transformation is efficient, it is the method of choice because of the facile and defined nature of the gene transfer.

A transgenic plant formed using Agrobacterium transformation methods typically contains a single gene on one chromosome. Such transgenic plants can be referred to as being heterozygous for the added gene. More preferred is a transgenic plant that is homozygous the added structural gene; i.e., a transgenic plant that contains 2 added genes, one gene at the same locus on each chromosome of a chromosome pair. A homozygous transgenic plant can be obtained by sexually mating (selfing) an independent segregant, transgenic plant that contains a single added gene, germinating some of the seed produced and analyzing the resulting plants produced for the gene of interest.

It is also to be understood that two different transgenic plants can also be mated to produce offspring that contain two independently segregating, exogenous genes. Selfing of appropriate progeny can produce plants that are homozygous for both added, exogenous genes that encode a polypeptide of interest. Back-crossing to a parental plant and out-crossing with a non-transgenic plant are also contemplated, as is vegetative propagation.

Transformation of plant protoplasts can be achieved using methods based on calcium phosphate precipitation, polyethylene glycol treatment, electroporation, and combinations of these treatments (see, for example, Potrykus et al., Mol. Gen. Genet., 205:193-200, 1986; Lorz et al., Mol. Gen. Genet., 199:178, 1985; Fromm et al., Nature, 319:791, 1986; Uchimiya et al., Mol. Gen. Genet., 204:204, 1986; Marcotte et al., Nature, 335:454-457, 1988).

Application of these systems to different plant strains depends upon the ability to regenerate that particular plant strain from protoplasts. Illustrative methods for the regeneration of cereals from protoplasts are described (Fujimura et al., Plant Tissue Culture Letters, 2:74, 1985; Toriyama et al., Theor. Appl. Genet., 205:34, 1986; Yamada et al., Plant Cell Rep., 4:85, 1986; Abdullah et al., Biotechnology, 4:1087, 1986).

To transform plant strains that cannot be successfully regenerated from protoplasts, other ways to introduce DNA into intact cells or tissues can be utilized. For example, regeneration of cereals from immature embryos or explants can be effected as described (Vasil, Biotechnology, 6:397, 1988). In addition, “particle gun” or high-velocity microprojectile technology can be utilized (Vasil et al., Bio/Technology, 10:667, 1992).

Using the latter technology, DNA is carried through the cell wall and into the cytoplasm on the surface of small metal particles as described (Klein et al., Nature, 328:70, 1987; Klein et al., Proc. Natl. Acad. Sci. (U.S.A.), 85:8502-8505, 1988; McCabe et al., Bio/Technology, 6:923, 1988). The metal particles penetrate through several layers of cells and thus allow the transformation of cells within tissue explants.

Other methods of cell transformation can also be used and include but are not limited to introduction of DNA into plants by direct DNA transfer into pollen (Hess et al., Intern Rev. Cytol., 107:367, 1987; Luo et al., Plant Mol. Biol. Reporter, 6:165, 1988), by direct injection of DNA into reproductive organs of a plant (Pena et al., Nature, 325:274, 1987), or by direct injection of DNA into the cells of immature embryos followed by the rehydration of desiccated embryos (Neuhaus et al., Theor. Appl. Genet., 75:30, 1987).

The regeneration, development, and cultivation of plants from single plant protoplast transformants or from various transformed explants is well known in the art (Weissbach and Weissbach, In: Methods for Plant Molecular Biology, Academic Press, San Diego, Calif., 1988). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene that encodes a protein of interest is well known in the art. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polypeptide is cultivated using methods well known to one skilled in the art.

There are a variety of methods for the regeneration of plants from plant tissue. The particular method of regeneration will depend on the starting plant tissue and the particular plant species to be regenerated.

Methods for transforming dicots, primarily by use of Agrobacterium tumefaciens and obtaining transgenic plants have been published for cotton (U.S. Pat. Nos. 5,004,863; 5,159,135; and 5,518,908); soybean (U.S. Pat. Nos. 6,384,301; 5,569,834; and 5,416,011; McCabe et al., Biotechnology, 6:923, 1988; Christou et al., Plant Physiol., 87:671-674, 1988); Brassica (U.S. Pat. No. 5,463,174); peanut (Cheng et al., Plant Cell Rep., 15:653-657, 1996; McKently et al., Plant Cell Rep., 14:699-703, 1995); papaya; pea (Grant et al., Plant Cell Rep., 15:254-258, 1995); and Arabidopsis thaliana (Bechtold et al., C.R. Acad. Sci. Paris, Life Sci., 316:1194-1199, 1993). The latter method for transforming Arabidopsis thaliana is commonly called “dipping” or vacuum infiltration or germplasm transformation. Transformation of monocotyledons using electroporation, particle bombardment and Agrobacterium have also been reported. Transformation and plant regeneration have been achieved in asparagus (Bytebier et al., Proc. Natl. Acad. Sci. (U.S.A.), 84:5354, 1987); barley (Wan and Lemaux, Plant Physiol., 104:37, 1994); maize (Rhodes et al., Science, 240:204, 1988; Gordon-Kamm et al., Plant Cell, 2:603-618, 1990; Fromm et al., Bio/Technology, 8:833, 1990; Koziel et al., Bio/Technology, 11:194, 1993; Armstrong et al., Crop Science, 35:550-557, 1995); oat (Somers et al., Bio/Technology, 10:1589, 1992); orchard grass (Horn et al., Plant Cell Rep., 7:469, 1988); rice (Toriyama et al., Theor Appl. Genet., 205:34, 1986; Part et al., Plant Mol. Biol., 32:1135-1148, 1996; Abedinia et al., Aust. J. Plant Physiol., 24:133-141, 1997; Zhang and Wu, Theor. Appl. Genet., 76:835, 1988; Zhang et al., Plant Cell Rep., 7:379, 1988; Battraw and Hall, Plant Sci., 86:191-202, 1992; Christou et al., BiolTechnology, 9:957, 1991); rye (DellaPenna et al., Nature, 325:274, 1987); sugarcane (Bower and Birch, Plant J., 2:409, 1992); tall fescue (Wang et al., Bio/Technology, 10:691, 1992); and wheat (Vasil et al., Bio/Technology, 10:667, 1992; U.S. Pat. No. 5,631,152).

Assays for gene expression based on the transient expression of cloned nucleic acid constructs have been developed by introducing the nucleic acid molecules into plant cells by polyethylene glycol treatment, electroporation, or particle bombardment (Marcotte et al., Nature, 335:454-457, 1988; Marcotte et al., Plant Cell, 1:523-532, 1989; McCarty et al., Cell, 66:895-905, 1991; Hattori et al., Genes Dev., 6:609-618, 1992; Goff et al., EMBO J., 9:2517-2522, 1990). Transient expression systems may be used to functionally dissect gene constructs (see generally, Maliga et al., Methods in Plant Molecular Biology, Cold Spring Harbor Press, 1995).

Any of the nucleic acid molecules of the present invention may be introduced into a plant cell in a permanent or transient manner in combination with other genetic elements such as vectors, promoters, enhancers, etc. Further, any of the nucleic acid molecules of the present invention may be introduced into a plant cell in a manner that allows for expression or overexpression of the protein or fragment thereof encoded by the nucleic acid molecule.

It is also to be understood that 2 different transgenic Brassica plants can also be mated to produce offspring that contain 2 independently segregating added, exogenous genes. Selfing of appropriate progeny can produce plants that are homozygous for both added, exogenous genes that encode a polypeptide of interest. Backcrossing to a parental plant and out-crossing with a non-transgenic plant are also contemplated, as is vegetative propagation.

Transgenic Brassica plants may find use in the commercial manufacture of proteins or other molecules, where the molecule of interest is extracted or purified from plant parts, seeds, and the like. Cells or tissue from the plants may also be cultured, grown in vitro, or fermented to manufacture such molecules.

The transgenic Brassica plants may also be used in commercial breeding programs, or may be crossed or bred to plants of related crop species. Improvements encoded by the recombinant DNA may be transferred, e.g., from cells of one species to cells of other species, e.g., by protoplast fusion.

The present invention also provides for a method of stably expressing a fatty acid synthase of interest in a Brassica plant, which includes, contacting the plant cell with a vector of the present invention that has a selectable marker gene and a nucleic acid encoding the fatty acid synthase of interest, under conditions effective to transform the plant cell. A promoter within the expression cassette can be any of the promoters provided herein, for example, a constitutive promoter, an inducible promoter, a tissue-specific promoter or a seed specific promoter. Such promoters can provide expression of an encoded fatty acid synthase at a desired time, or at a desired developmental stage, or in a desired tissue.

The present invention also provides for a method of stably expressing a fatty acid synthase of interest in a plant, which includes, contacting the plant cell with a vector of the present invention that has a nucleic acid encoding the fatty acid synthase of interest, under conditions effective to transfer and integrate the vector into the nuclear genome of the cell. The vector can also include a selectable marker gene. When using the vector with Agrobacterium tumefaciens, the vector can have an Agrobacterium tumefaciens origin of replication.

In another embodiment, the present invention provides a method of producing a Brassica oil, comprising the steps of: a) growing an oilseed Brassica plant, the genome of which contains a nucleic acid molecule encoding a multifunctional fatty acid synthase, to produce oil-containing seeds; and b) extracting oil from the seeds. In another embodiment, the present invention provides a method of producing a Brassica oil, comprising the steps of: a) growing an oilseed Brassica plant, the genome of which contains a nucleic acid molecule encoding a phosphopantetheine:protein transferase, to produce oil-containing seeds; and b) extracting oil from the seeds.

Plants: Plants for use with the vectors of the present invention include Brassica sp., particularly those Brassica species useful as sources of seed oil (e.g., B. napus, B. rapa, B. juncea).

The following examples are provided to illustrate the present invention and are not intended to limit the present invention in any way.

### EXAMPLE 1

This example describes the isolation of the fasA and pptl genes from Brevibacterium ammoniagenes.

Genomic DNA was isolated from B. ammoniagenes (ATCC 6871) using standard methodologies. A genomic library was prepared by partially digesting B. ammoniagenes genomic DNA with the restriction enzyme Sau3A, isolating DNA fragments ranging from 30-42 kb in size and generating the library using the SuperCos 1 Cosmid Vector kit from Stratagene, Inc. (La Jolla, Calif.). The genomic library was screened by hybridization and washing under stringent conditions with a ³²P-labelled 1.1 kb fasA PCR fragment generated from isolated genomic DNA using the following PCR primers:

| | |
|---|---|
| 14713 (forward): | |
| 5′-CCAGCTCAACGATGAAGTAG-3′ | (SEQ ID NO: 5) |
| and | |
| 14714 (reverse): | |
| 5′-TCGATGATCTGGTCTACTTC-3′. | (SEQ ID NO: 6) |

Prehybridization was in a solution of 40% formamide, 5×SSC, 50 mM sodium phosphate, pH 7.0, 5× Denhardt's, 0.1% SDS, 5 mM EDTA, 0.1 1.4, g/ml salmon sperm DNA, and 5% Dextran sulfate for 2 hrs at 42° C. and hybridization was in the same solution as described overnight at 42° C. The filters were rinsed briefly in 0.1×SSC, 0.1% SDS at RT and then washed 2 times for 20 min each in 0.1×SSC, 0.1% SDS at 50° C. FasA-containing clones were identified by autoradiography and restriction mapping. Selected cosmid clones were analyzed in more detail and one clone was confirmed to have the full-length fasA gene by restriction mapping and comparison with the restriction sites in the published sequence (Stuible et al., J. Bacteriol., 178:4787-4793, 1996).

The full-length fasA gene was assembled so as to introduce convenient flanking restriction sites for sub-cloning by using the following basic steps: a) PCR amplification of the 5′ and 3′ ends; b) assembling the 5′ and 3′ ends of the gene together by an overlapping PCR strategy resulting in deletion of the fasA sequence between the internal MfeI and XhoI sites; c) cloning the “5′-3′ fused” PCR fragment; d) insertion of the 8166 bp fasA MfeI/XhoI fragment between the MfeI and XhoI sites in the “5′-3′ fused” PCR fragment so as to regenerate the full-length fasA gene with convenient flanking cloning sites. The details for each of these steps are outlined below.

A 5′ 280 bp fasA PCR fragment was generated using the following primers:

16393 (forward)

(SEQ ID NO: 9)

5′-TCTAGATGCATAGTTAACATGTCGTTGACCCCCTTGC-3′
and
14873 (reverse)

(SEQ ID NO: 10)

5′-GGTACGCGTCATATTCCTTG-3′

The forward primer, 16393, introduced XbaI, NsiI, HpaI, and PciI flanking restriction sites.

A 3′ 946 bpfasA PCR fragment was generated using the following primers:

16385 (forward)

(SEQ ID NO: 11)

5′-CAAGGAATATGACGCGTACCCTCGAGGCAGAAGGCGGCGG-3′
and
16394 (reverse)

(SEQ ID NO: 12)

5′-ATGCATGTTAACATGTCTACTTTGTCCTACTTCGCCG-3′

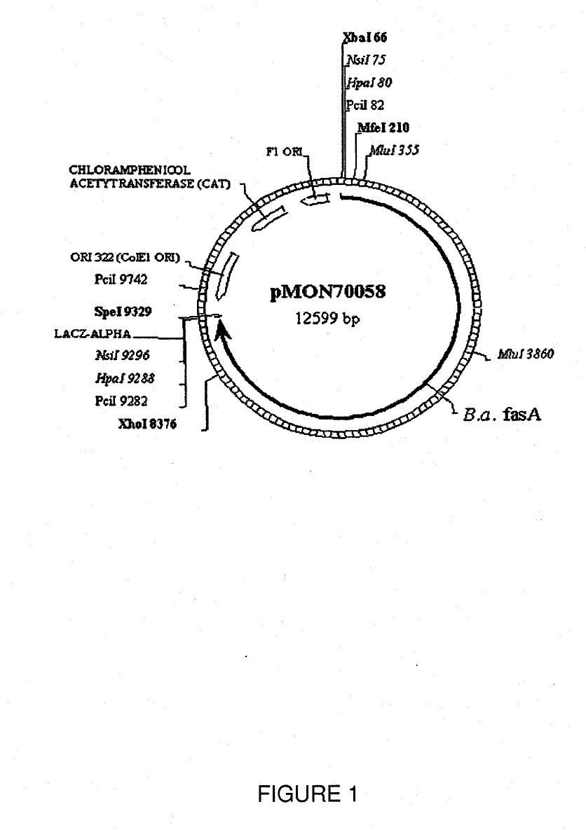

The reverse primer, 16394, introduced 3′ flanking NsiI, HpaI, and PciI restriction sites. The forward primer, 16385, contained 20 bp of sequence matching the 3′-end of the 5′ 280 bp restriction fragment described above to allow the 2 fragments to anneal together. The 5′ 280 bp fasA PCR fragment and the 3′ 946 bp fasA PCR fragment were fused together by annealing the 2 fragments and PCR amplifying the full length (1206 bp) overlapped fragment using the external primers 16393 (forward) and 16394 (reverse). The 1206 bp 5′-3′-fused PCR fragment was cloned into pCR-Blunt II-TOPO (Invitrogen Corporation, Carlsbad, Calif.) and the correct DNA sequence was confirmed by sequencing. The 1206 bp 5′-3′-fused PCR fragment was then sub-cloned as an SpeI/XbaI fragment into a Bluescript pBC KS+ (Stratagene Inc., La Jolla, Calif.) vector which contained a modified multiple cloning sequence (pCGN3686). The full-length fasA gene was then obtained by ligation of the 3505 bp MluI/MluI and 4516 bp MluI/XhoI internal fasA fragments isolated from the full-length cosmid clone between the MluI and XhoI sites in the 5′-3′ fused PCR fragment to make pMON70058 (FIG. 1).

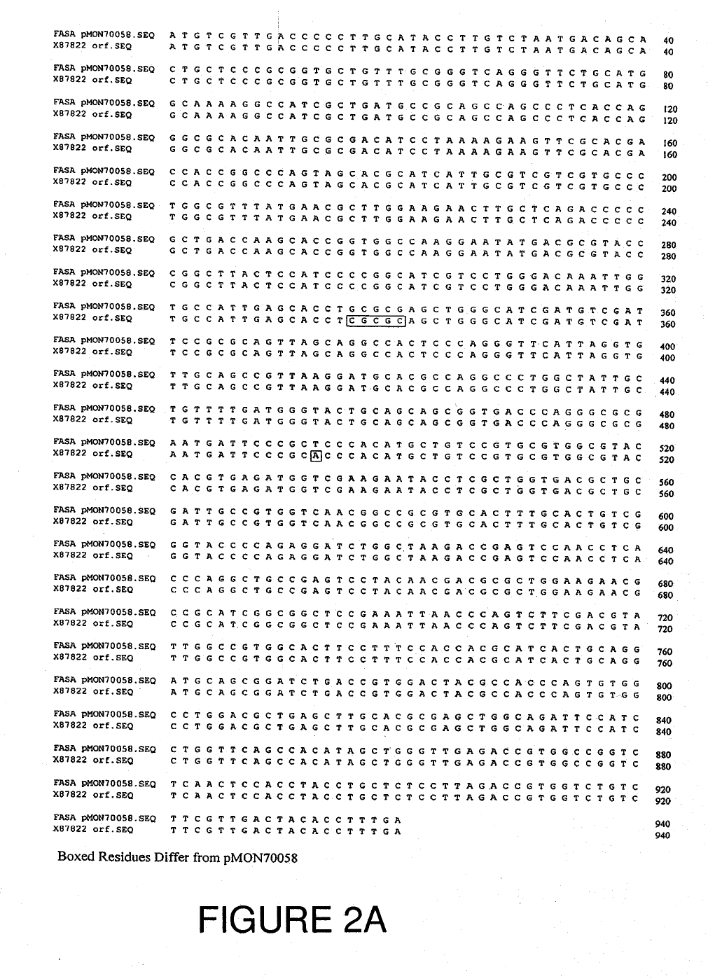

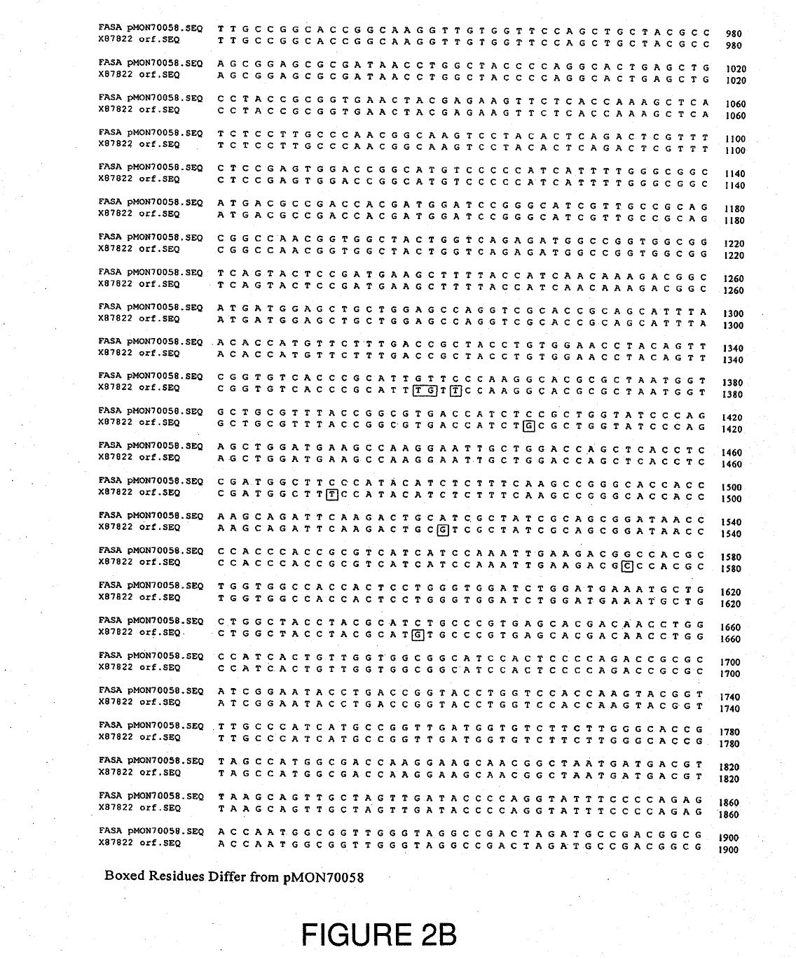

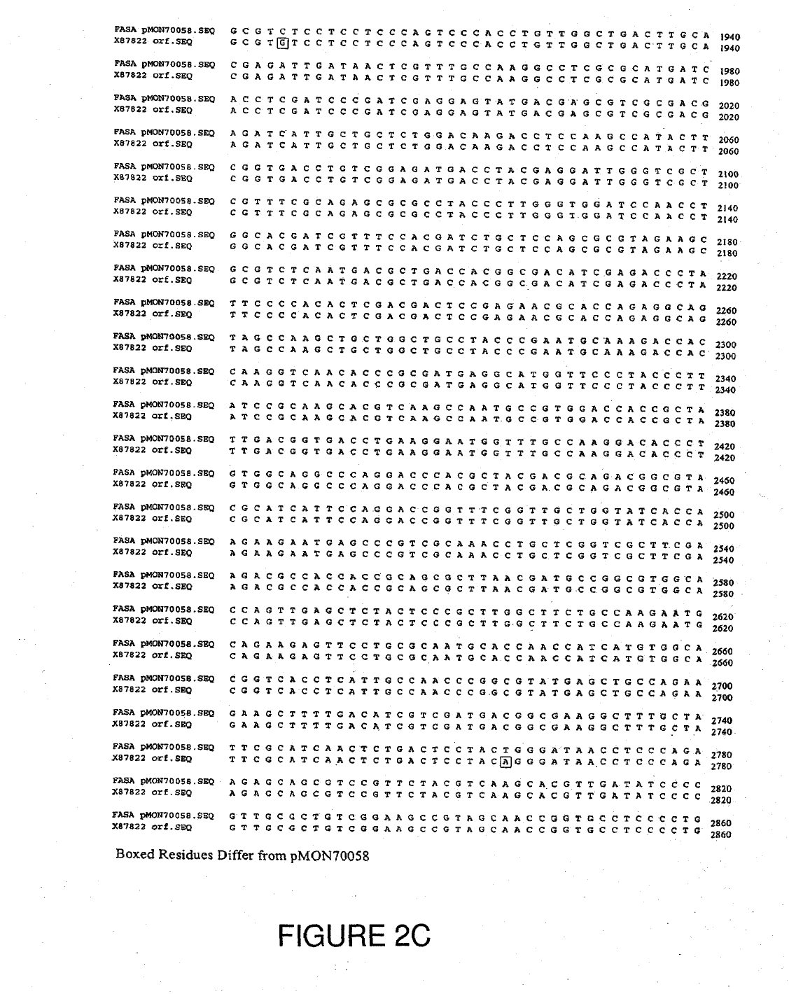

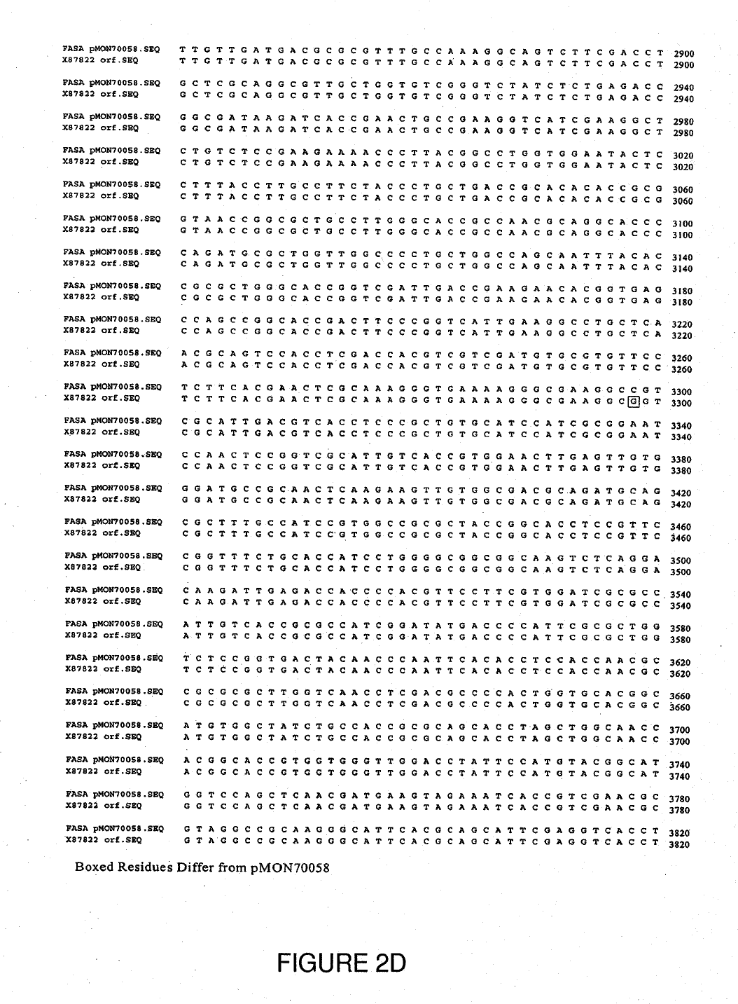

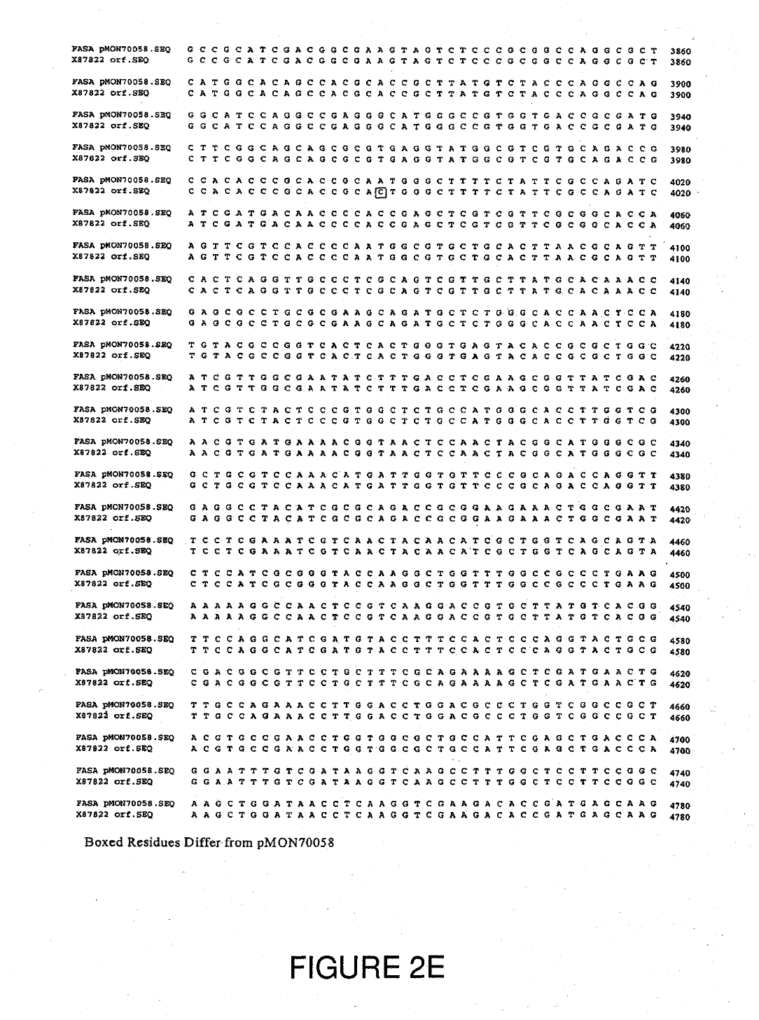

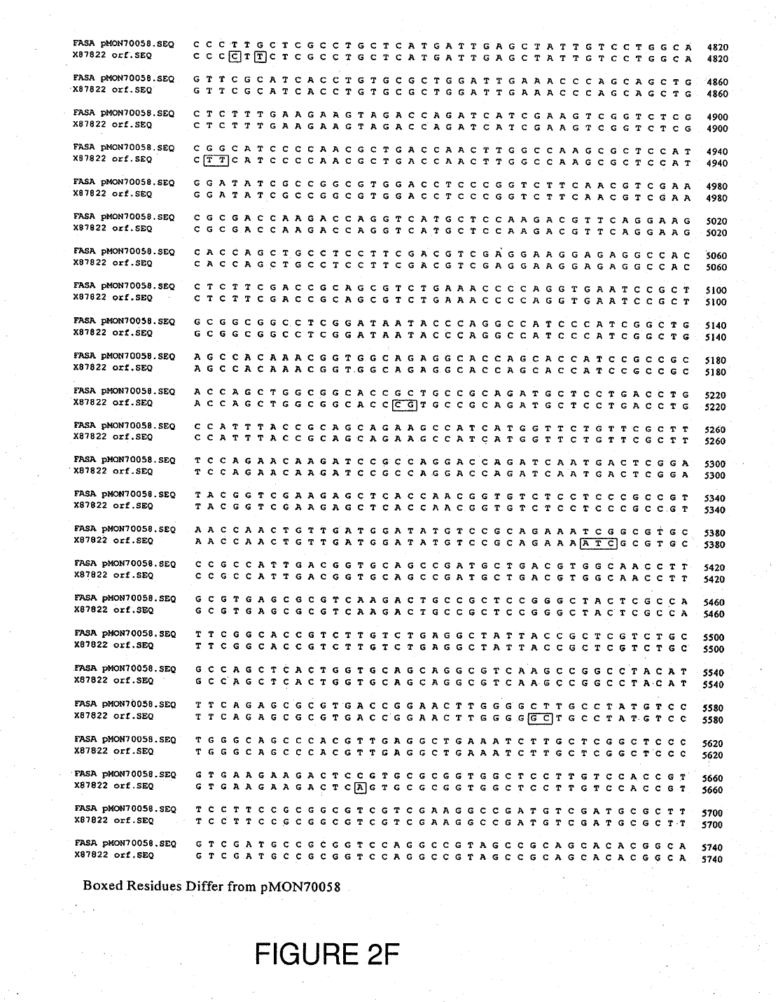

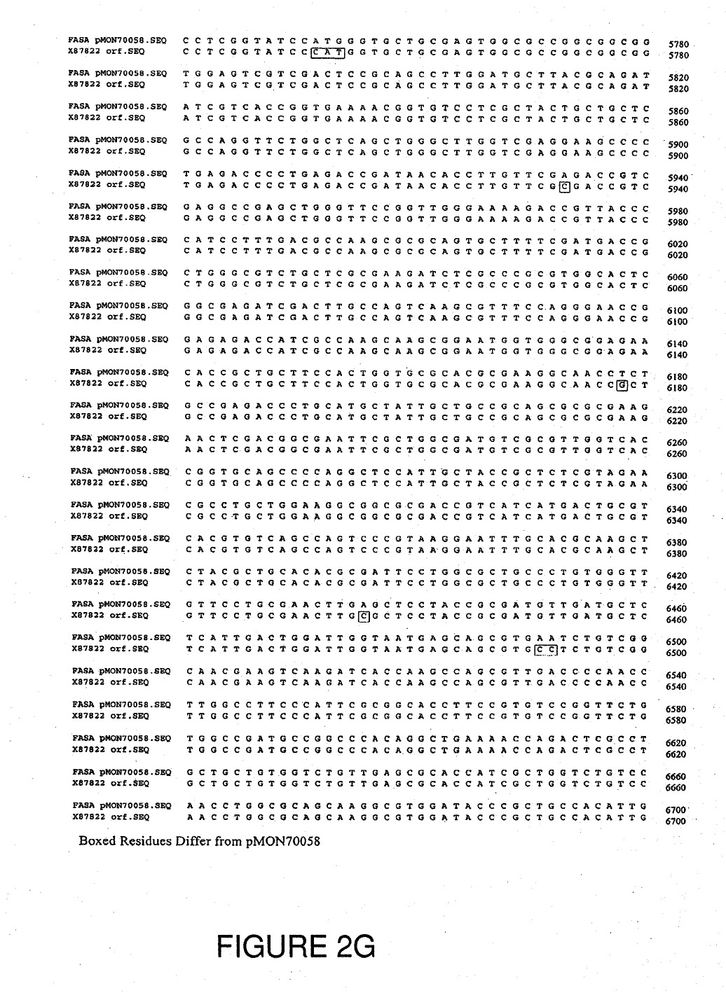

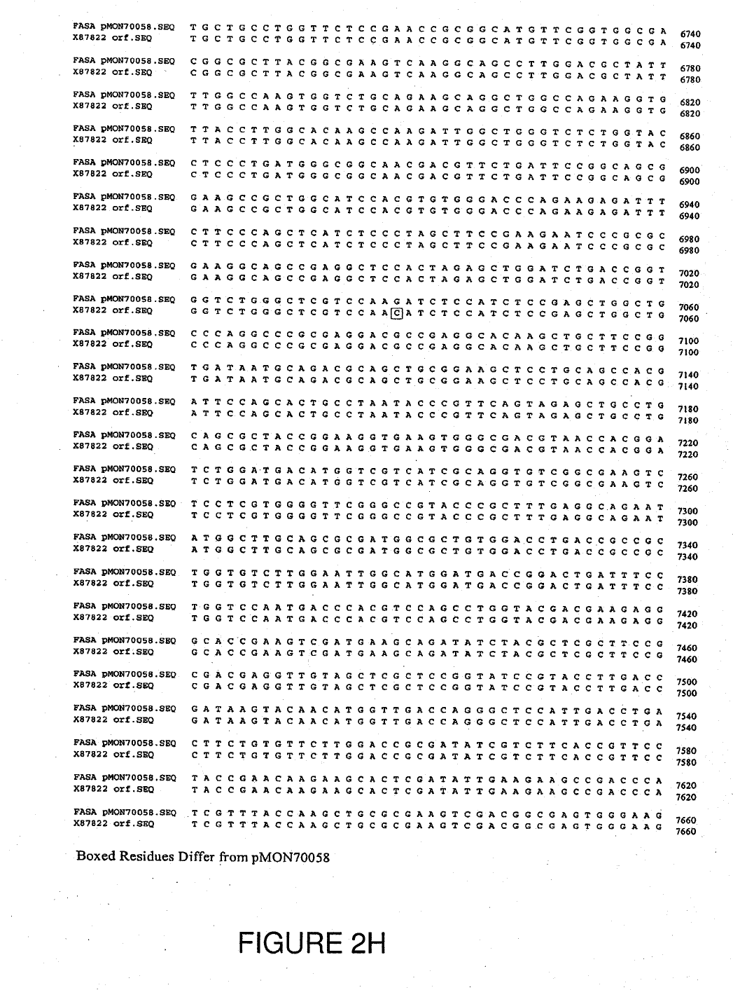

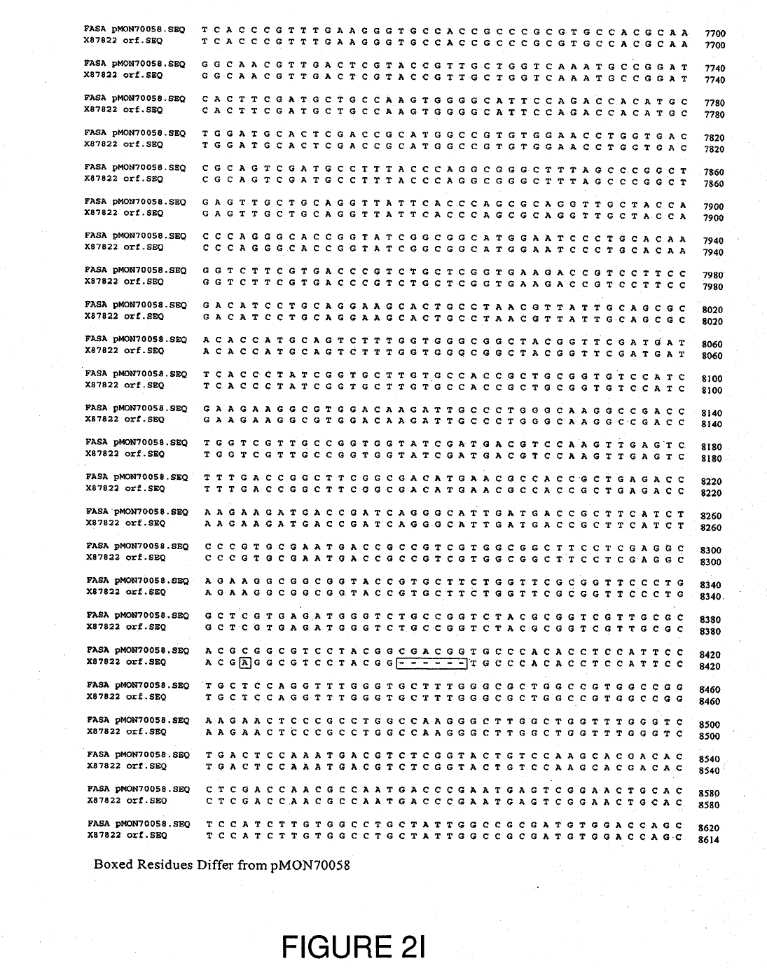

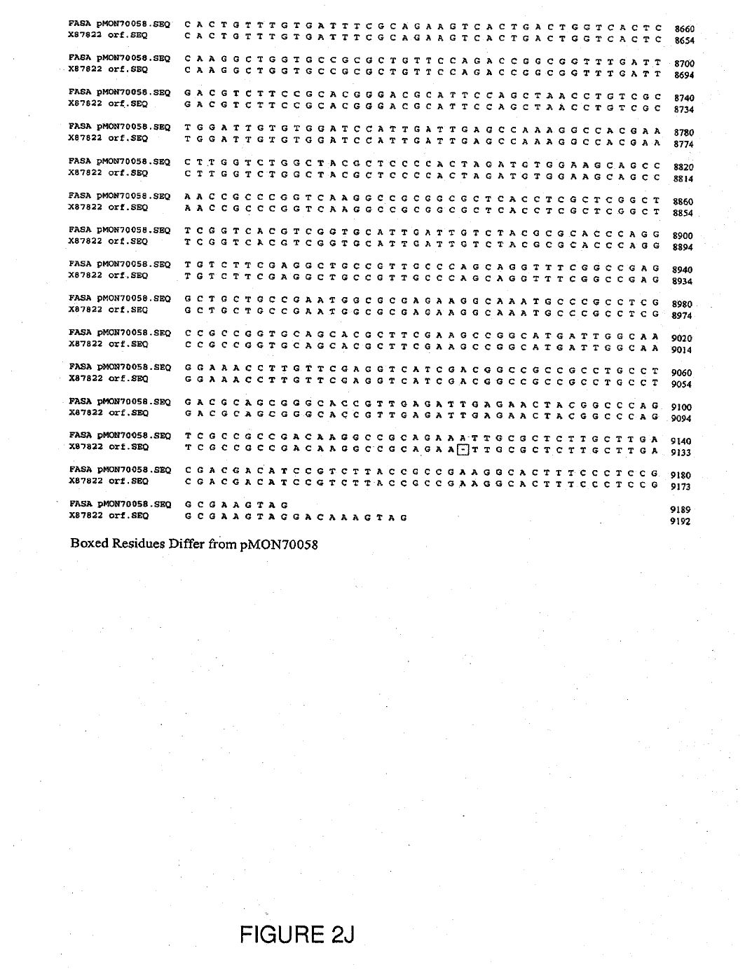

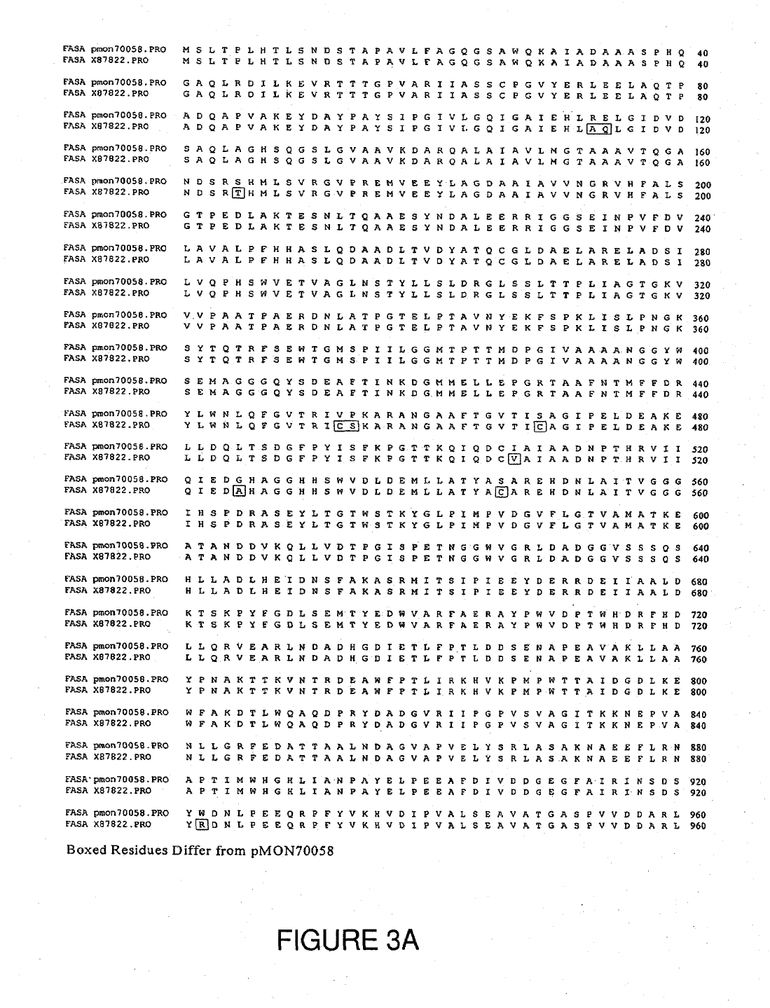

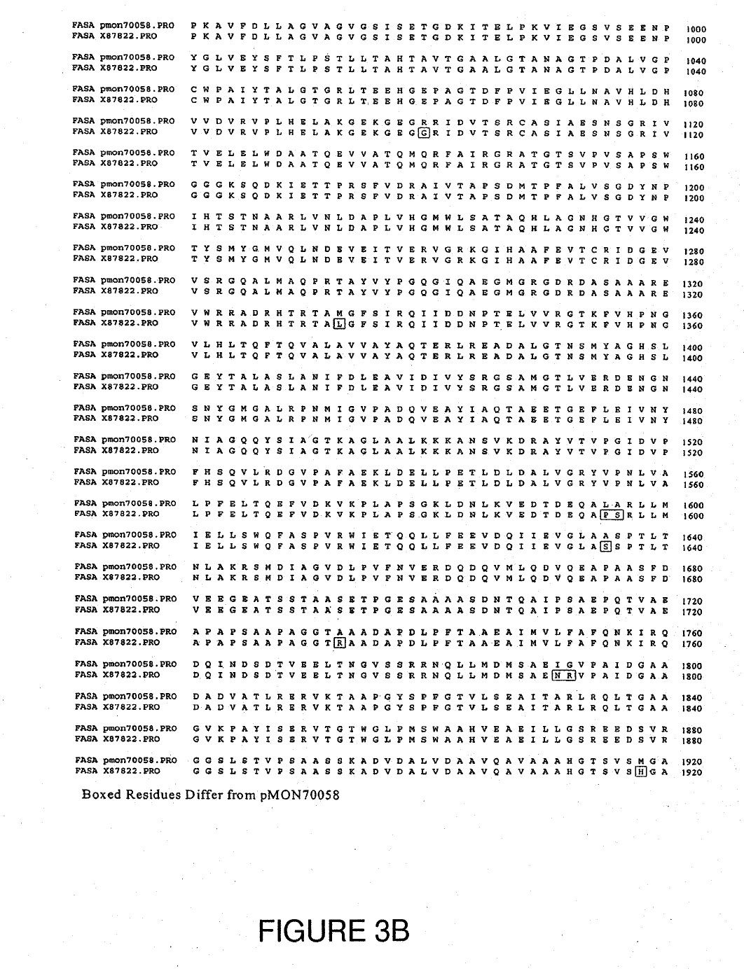

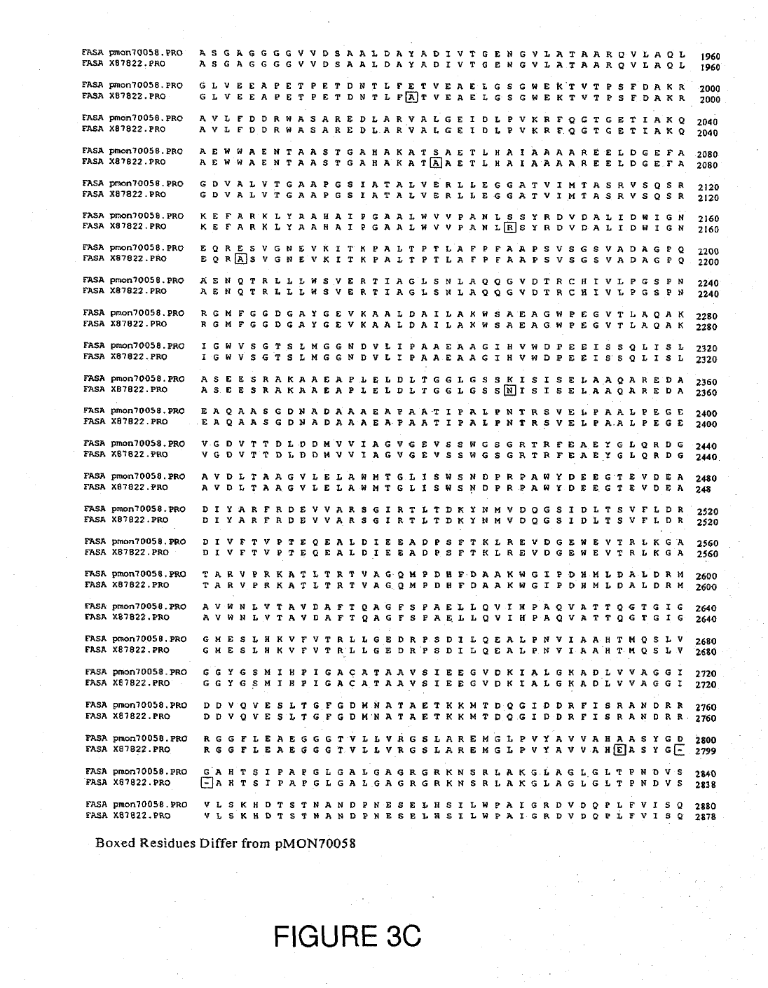

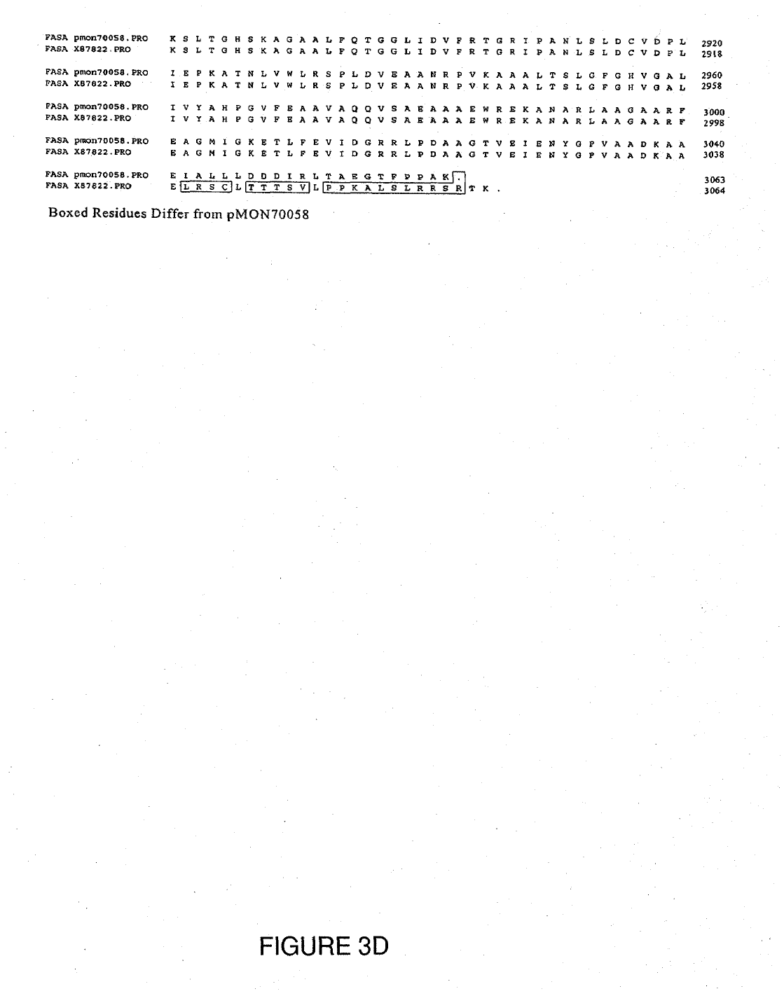

The complete double-stranded sequence of the full-length fasA gene open reading frame in pMON70058 was determined using a Perkin Elmer ABI 377 DNA sequencer (SEQ ID NO: 1). The corresponding protein sequence (SEQ ID NO: 2) was predicted based on standard genetic code using the program Omiga (Accelrys, Inc., Cambridge, UK) and compared with the published FAS A sequence (FIG. 2). Alignment of both the nucleic acid and predicted amino acid sequences to the published sequences (Stuible et al., J. Bacteriol., 178:4787, 1996) revealed a number of differences both at the DNA and protein levels (FIGS. 2 and 3).

The B. ammoniagenes pptl gene was PCR amplified from isolated genomic DNA using the following primers:

16117 (forward):

(SEQ ID NO: 7)

5′-GTCGACATGCTCGACAACCGTGAAGCG-3′
and
16118 (reverse):

(SEQ ID NO: 8)

5′-AGATCTTCACTGGTGGCTTGCCGTAGATCGC-3′

The PCR-amplified fragment was then cloned into the commercially available cloning vector pCR-Blunt II TOPO (Invitrogen Corporation, Carlsbad, Calif.). The complete double stranded sequence of the full-length pptl gene (SEQ ID NO: 3) was determined using a Perkin Elmer ABI 377 DNA sequencer. The corresponding protein sequence (SEQ ID NO: 4) was predicted based on standard genetic code using the program Omiga and compared with the published pptl sequence. Alignment of both the nucleic acid and predicted amino acid sequences to the published sequences (Stuible et al., J. Bacteriol., 178:4787, 1996) revealed that the cloned pptl gene was identical to the published sequence.

### EXAMPLE 2

This example describes the transformation of E. coli with fasA gene constructs for functional testing.

The full-length, sequence-confirmed B. ammoniagenes pptl gene in the pCR-Blunt II TOPO vector described in Example 1 was cut out of the pCR-Blunt II TOPO backbone as a SalI/BglII fragment, and ligated into the SalI/Bam_HI sites, respectively, of pSU19 (Bartolome et al., Gene, 102(1):75-78, 1991). The Sal I sites of both the pptl and the pSU19 fragments were blunt-ended with the Kienow fragment of DNA polymerase I prior to ligation to enable in-frame insertion of the pptl coding sequence into the lacZ coding sequence of pSU19. The PPT1 protein was thus expressed in E. coli as a lacZ fusion protein upon induction of the lacZ promoter in pSU19 by the use of isopropyl-1-thio-β-D-galactopyranoside (IPTG). This pptl-containing vector was then transformed into E. coli strain VCS257 from Stratagene (cat#200256-51), along with the mfFAS cosmid clone, described in Example 1, for functional testing.

Because the plasmid pSU19 has a pACYC184 origin of replication and conveys chloramphenicol resistance, the pptl expressing plasmid could be stably maintained along with the cosmid (ampicillin resistance) expressing the fasA gene.

Based on the published report of Stuible et al., Eur. J. Biochem., 248:481-487 (1997), the endogenous fasA promoter was used to express the mfFAS polypeptide encoded by fasA in E. coli. As a result, E. coli transformants containing the fasA cosmid alone, the pSU19/pptl construct alone, and both the fasA cosmid and the pSU19/pptl construct were made for functional testing. The full-length fasA gene was also subcloned as a PciI fragment from pMON70058 into the E. coli expression vector pQE60 (QIAGEN, Inc., Valencia, Calif.) to enable inducible expression from an E. coli promoter (pMON70081).

### EXAMPLE 3

This example sets forth the functional testing of transgene activity in E. coli using enzymatic assays.

In order to assay the E. coli strains containing the fasA cosmid and pptl gene construct the fasA gene product was partially purified essentially as outlined in Kawaguchi et al., Methods in Enzymology, 71:120-127 (1981). Frozen cells from the strain containing either the fasA cosmid alone, the pSU19/pptl construct alone, both the fasA cosmid and the pSU19/pptl construct, or the untransformed cell line alone were thawed in 0.1M potassium phosphate buffer (−1 ml/1 gm) and cells lysed by high speed mixing with glass beads. The supernatant was centrifuged at 105,000×g for 60 minutes and removed. Ammonium sulphate was slowly added to the supernatant to give a final concentration of 30% w/v followed by 30 minutes of stirring. A second centrifugation step (25,000×g) was performed and the precipitate was re-suspended in 0.5M potassium phosphate buffer before passing through a Sephadex G-25 column.

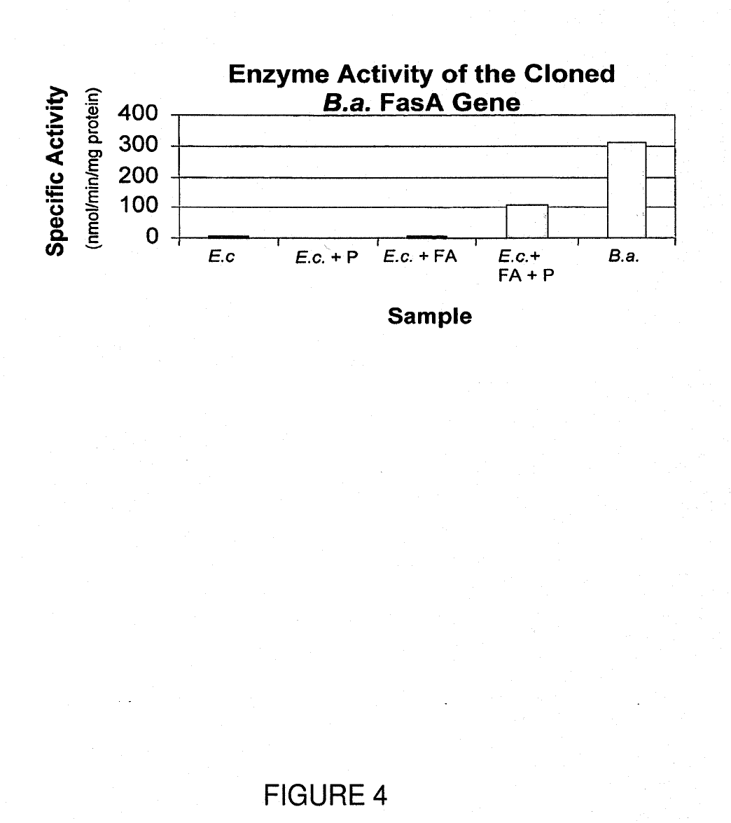

The fasA activity in each of the extracts was determined by a radiochemical assay at 37° C. for 15 minutes using the conditions outlined in Kawaguchi et al., (1981). The results of these assays (shown in FIG. 4) demonstrated that only when the fasA cosmid (FA) and the pSU19/pptl (P) construct were both present was there any measurable fasA activity. Furthermore, they demonstrated that the fasA gene that was cloned and used for preparation of Brassica transformation constructs did encode a functional fasA enzyme.

### EXAMPLE 4

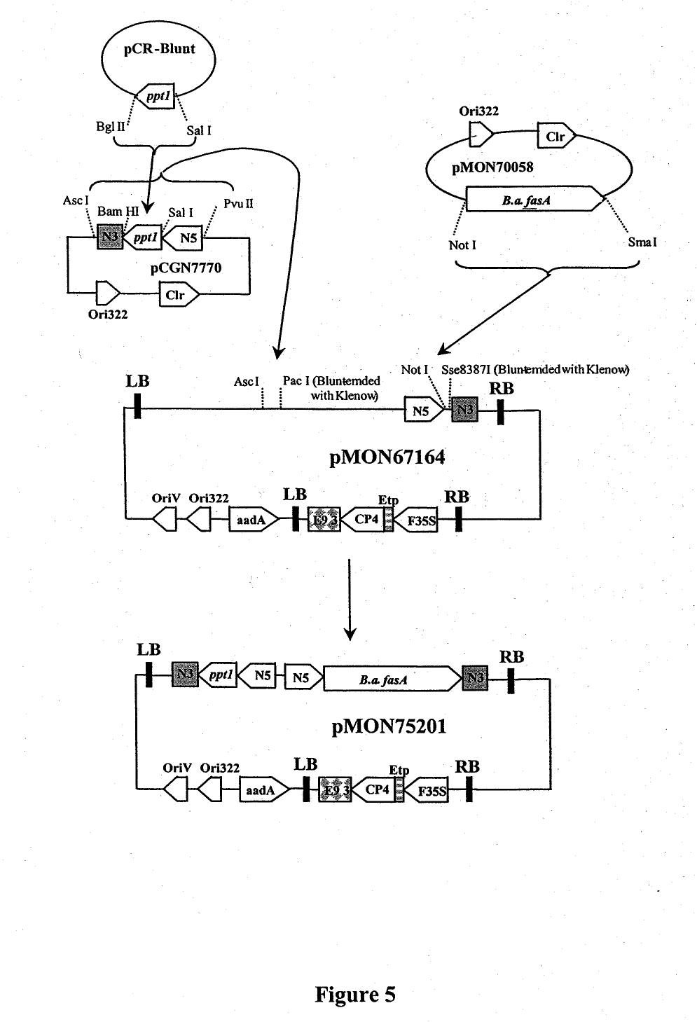

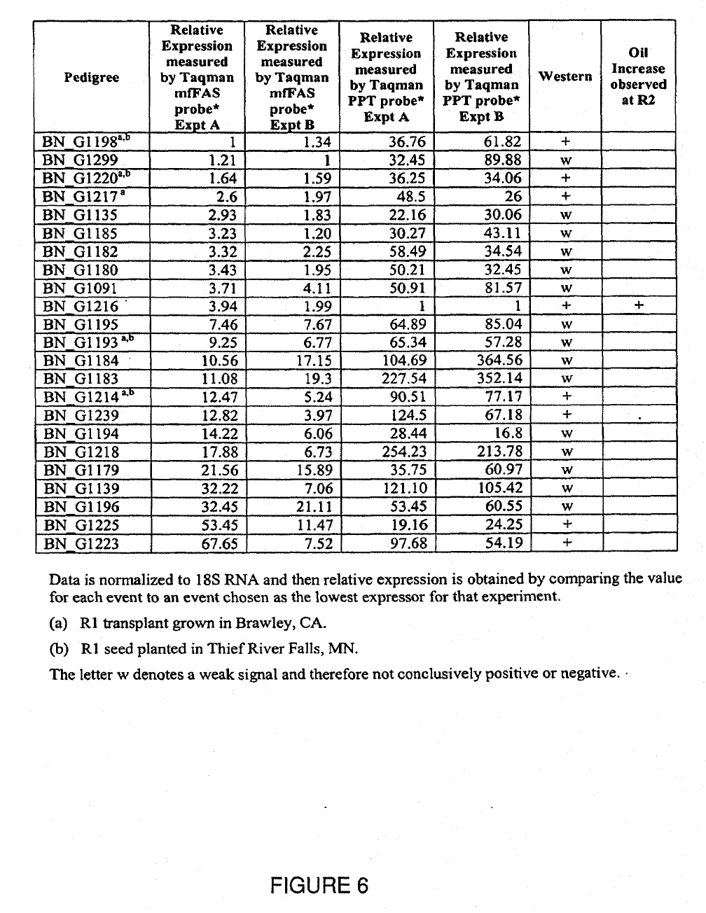

This example describes the construction of a plant binary vector for seed-specific expression of the fasA gene in canola plants. The construction is shown graphically in FIGS. 5 and 6. The vector pMON75201 was designed to produce seed-specific expression of the B. ammoniagenes fasA and pptl genes in canola.

The full-length, sequence-confirmed B. ammoniagenes pptl gene in the pCR-Blunt II TOPO vector described in Example 1 was cut out of the pCR-Blunt II TOPO backbone using the Sal I and Bgl II sites engineered into the PCR primers 16117 and 16118 (SEQ ID NOs: 7 and 8, respectively) used in the cloning and ligated to the Sal I and BamHI sites between the napin promoter (base pairs 407-2151 of the Brassica campestris napin gene, N5, GenBank Accession Number M64632) and the napin 3′ untranslated region (UTR), N3, (base pairs 2728-3982 of the Brassica campestris napin gene, GenBank Accession Number M64632) found in the plant/E. coli binary vector pCGN7770 (FIG. 5). The napin promoter/B. ammoniagenes pptil napin 3′ UTR cassette was combined with the B. ammoniagenes fasA gene for simultaneous expression in Brassica plants as described below.

The full-length, sequence-confirmed B. ammoniagenes fasA gene was removed from pMON70058 (described in Example 1 and FIG. 1) using the restriction enzymes NotI and SmaI and was ligated into the NotI and blunted Sse8387I restriction sites between a napin promoter and napin 3′ UTR (as described above) contained in a two T-DNA binary vector pMON67164. The Sse8387I site was blunt-ended by the action of Klenow fragment of DNA polymerase I. The resultant vector, containing the pMON67164 backbone and the B. ammoniagenes fasA gene flanked by the napin expression sequences, was digested with PacI, blunt-ended by the action of Klenow fragment of DNA polymerase I, and then digested with AscI. The AscI/PvuII fragment containing the napin promoter/B. ammoniagenes pptl gene/napin 3′ UTR cassette in pCGN7770 (described above) was then inserted into the Pad blunt/AscI sites to form pMON75201. pMON75201 is a two T-DNA vector Containing both the B. ammoniagenes fasA gene and the B. ammoniagenes pptl gene each under the control of seed-specific napin expression sequences (napin promoter and 3′ UTR) and located within one set of T-DNA left and right borders. A selectable marker for plant transformation, containing the FMV 35S promoter, (F35S, base pairs 6927-6474 of the FMV promoter which is the promoter for ORF VII, GenBank Accession Number X06166) driving a CP4 selectable marker gene (a chloroplast targeting sequence from the Arabidopsis EPSP gene linked to a synthetic EPSP synthase coding region as described in U.S. Pat. No. 5,633,435) and a E9 3′ UTR (Coruzzi et al., EMBO J., 3(8):1671-1679, 1984) is located within a second set of T-DNA left and right borders.

### EXAMPLE 5

This example describes the transformation of canola plants with fasA and pptl genes. Canola plants (Brassica napus) are transformed using a modification of the protocol described by Radke et al., Plant Cell Reports, 11:499-505 (1992). Briefly canola seed of the cultivar ‘Ebony’ (Monsanto Canada, Inc., Winnipeg, Canada) are disinfected and germinated in vitro as described in Radke et al., 1992. Precocultivation with tobacco feeder plates, explant preparation and inoculation of explants with Agrobacterium tumefaciens strain ABI (Koncz and Schell, Mol Gen Genet., 204:383-396, 1986) containing the vector pMON75201 are as described with the Agrobacterium being maintained in LB media (solid or liquid) containing 75 mg/l spectinomycin, 25 mg/l chloramphenicol, and 50 mg/l kanamycin. For plant transformation including callus induction, shoot regeneration, maturation and rooting, glyphosate selection is used rather than the kanamycin selection as described in Radke et al., 1992. Specifically, the B5-1 callus induction medium is supplemented with 500 mg/l carbenicillin and 50 mg/l Timentin (Duchefa Biochemie BV) to inhibit the Agrobacterium growth and kanamycin is omitted from the media. B5BZ shoot regeneration medium contains 500 mg/l carbenicillin, 50 mg/l Timentin, and 45 mg/l glyphosate with explants being transferred to fresh medium every 2 weeks. Glyphosate selected shoots are transferred to hormone-free B5-0 shoot maturation medium containing 300 mg/l carbenicillin and 45 mg/l glyphosate for 2 weeks and finally shoots are transferred to B5 root induction medium containing 45 mg/l glyphosate. Rooted green plantlets are transplanted to potting soil and acclimated to green house conditions. Plants are maintained in a greenhouse under standard conditions.

Developing seed is harvested at various stages after pollination and stored at −70° C. Mature seed is collected and stored under controlled conditions consisting of about 17° C. and 30% humidity.

### EXAMPLE 6

This example describes the evaluation and selection of R2 seed from canola plants transformed with the fasA and pptl genes as described above in Example 5.

From the transformation of canola ex-plants with pMON75201, as described above, 110 events were generated. These events were analyzed for the presence of the gene of interest (GOI) by PCR, for transcription expression of the GOI by TaqMan methodology, and for the presence of the FasA protein by western blot analysis. The events testing positive for the GOI by PCR were considered for selection to advance in the development of high oil varieties.

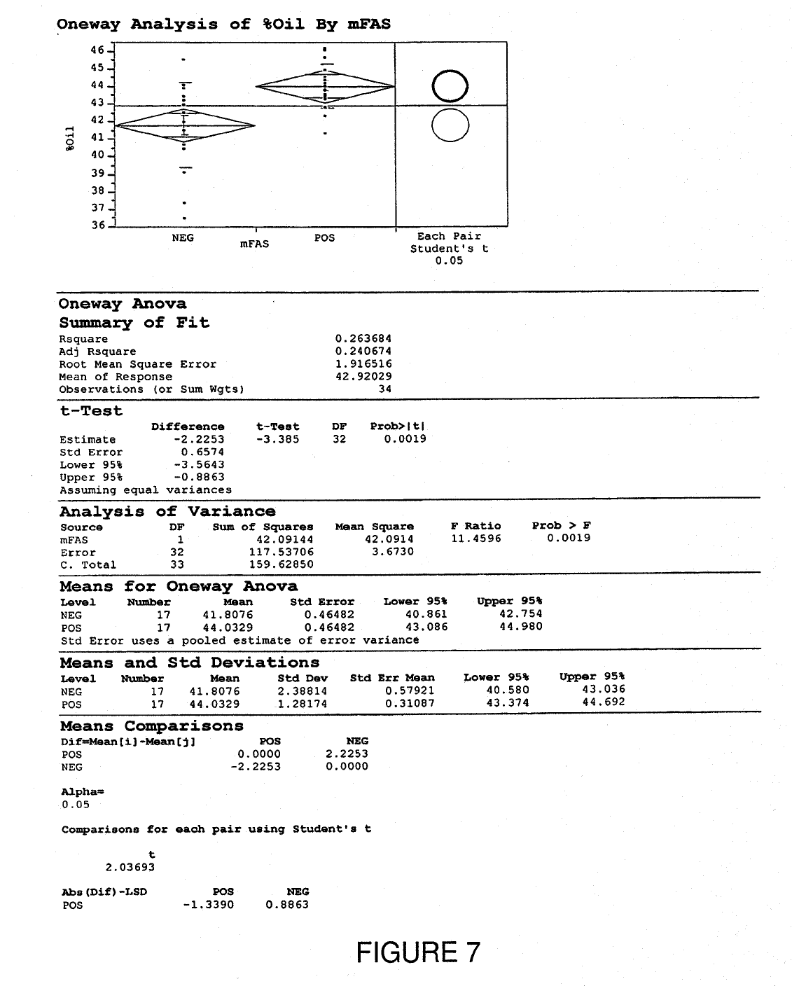

The western blot analysis for the presence of the FasA protein was done using methods well known in the art. Briefly, antibodies were generated by a contract laboratory (Zymed Laboratories Inc., South San Francisco, Calif.) to 4 synthetic peptides located in different regions of the fasA gene; fasA 2843-2858=(C)SKHDTSTNANDPNESE (SEQ ID NO: 44),fasA 1755-1768=(C)QNKIRQDQINDSDT (SEQ ID NO: 45), fasA 915-930=(C)RINSDSYWDNLPEEQR (SEQ ID NO: 46), and fasA 1431-1444=(C)TLVERDENGNSNYG (SEQ ID NO: 47). A protein extract from each of the events was separated using SDS-PAGE according to Laemmli, Nature, 227: 680 (1970), and transferred to a polyvinylidene difluoride (PVDF) membrane in Tris buffered saline (TBS; 25 mM Tris, 150 mM NaCl) (BioRad, Bulletin #9016). The membrane was blocked with TBST (TBS with 0.05% Tween 20) containing 1% bovine serum albumin (BSA) for 10 minutes then incubated overnight at room temperature with a combined solution of primary antibodies from fasA 2843_{—}2858, fasA 1755-1768, and fasA 1431_{—}1444, at a 1:2000 dilution of each antibody in TBST with 1% BSA. The membrane was washed 3×15 minutes with TBST then exposed to a reporting, secondary antibody (Anti-rabbit-AP conjugate, Promega S3731, Madison, Wis., 1:5000 in TBST) for one hour. The membrane was washed 3×15 minutes with TBST followed by 2 minutes with TBS to remove residual Tween 20. Western Blue Stabilized Substrate for Alkaline Phosphatase (Promega S3841, Madison, Wis.) was added to visualize protein. Development was stopped by rinsing the membrane with purified water. Of the events tested, 23 were determined to be potentially western positive by exhibiting at least a weak response, and 8 of those confirmed to be positive by exhibiting a strong response. The results are shown in FIG. 7.

Mature R1 seed from all 23 events were planted and a selection test was performed to identify gene positive and gene negative lines for each event. For the event selection test, plants from each event were analyzed for the presence of the gene of interest (GOI) by PCR using primers from the GOI and promoter region. DNA for the PCR was isolated from leaf tissue using DNeasy 96 Plant Kit from Qiagen (Cat. No. 69181). The forward primer, located in the promoter region, was primer #6456 (5′-TTCATAAGATGTCACGCCAGG-3′) (SEQ ID NO: 48), and the reverse primer, located in the GOI, was primer #14873 (5′-GGTACGCGTCATATTCCTTG-3′) (SEQ ID NO: 49). PCR protocol was set at 97° C. for 1 minute, 40 cycles of: {94° C. 15 seconds, 60° C. 30 seconds, 72° C. 30 seconds}, and 72° C. for 5 minutes. Fourteen events (indicated by a in FIG. 7) were advanced to the R2 seed stage by growing R1 plants to maturity in the greenhouse under standard conditions. Ten of these events were identified as single locus (indicated by ^{b} in FIG. 7) and were also planted in field trials (Brawley, Calif.) as R1 transplants. The R2 seed from gene positive and null segregants from 4 events (BN_G1193, BN_G1198, BN_G1214, and BN_G1220) as well as commercial control lines, were planted in a randomized complete bloc experiment in field trials (Thief River Falls, Minn.).

In addition to the PCR gene confirmation and the western blot analysis, developing R1 seed (approximately 20/event) of the 23 events were analyzed for transcript expression of the genes of interest, fasA and pptl, by TaqMan. TaqMan analysis was performed using the TaqMan One-Step RT-PCR Master Mix Reagents Kit and Protocol (#4310299 rev. C, Applied Biosystems, Foster City, Calif.). Low, medium, and high GOI-expressing lines are carried forward. Two separate experiments were performed on the same, pooled sample from each event. The results of both experiments are shown in tabular form in FIG. 7.

On the occasion when multiple gene copies of the GOI or marker are present, additional work is required to generate a null segregant from which phenotypic comparisons can be made. In addition to the original 14 events that were advanced, two GOI multicopy events (BN_G1223 and BN_G1239) were identified that exhibited a strong response in the western blot analysis. These events were crossed to the variety Ebony in order to produce gene positive as well as null segregants from an F1 population. Event BN_G1216 contained multiple copies of the marker gene, therefore it was out-crossed to Ebony as well. F1 transplants were generated for these events and were planted in the field and greenhouse. These 3 events are identified by ^{C} in FIG. 7. A comparison of the gene positive and null segregant selections is made in the F2 seed generation.

Oil and protein content of the F1 and R2 seed were established by near-infrared reflectance (NW) spectroscopy (Williams and Norris, eds., Near-infrared Technology in the Agricultural and Food Industries, American Association of Cereal Chemists, Inc., St. Paul, Minn. (1987)), using a standard curve generated from analysis of canola seed with varying oil and protein levels. Briefly, mature canola seeds previously dried to less than 10% moisture were equilibrated to ambient humidity in paper envelopes at room temperature. Single replicate sub-samples (2-3 g) were placed in NW ring cups (aluminum/quartz; 2 inch diameter by 0.5 inch thick, Foss North America Inc., Silver Springs, Md.) and sealed with a paperboard disk. The loaded ring cups were placed in an autoloader and scanned sequentially on a Foss Analytical model 6500 Spectrometer, (Foss North America Inc., Silver Springs, Md.). Each sample was scanned 25 times from 400 to 2500 nm (resolution 2 nm) and the average spectrum was compiled. The averaged spectrum was reduced to second derivative spectra, smoothed, and transformed to a series of principal component scores. The total oil and protein levels were predicted based on a previously prepared calibration models.

Commercially available software (WinISI ver 1.00, Infrasoft International LLC, State College, Pa.) was used for calibration development and instrument operation.

One-way analysis of variance and the Student's T-test (JMP software, version 4.04, SAS Institute Inc., Cary, N.C.) was performed to identify significant differences between transgenic and non-transgenic seed pools as determined by transgene-specific PCR.

As a result of the statistical analysis of the oil results, R2 seed from event BN_G1216 was determined to have a statistically significant increase in oil content as compared to a negative isoline control. The mean oil content determined by a one-way analysis of variance (ANOVA) for the positive isoline was 44.0%, as compared to 41.8% for the negative isoline control. The results are shown in FIG. 8.

### EXAMPLE 7

This example describes the generation and evaluation of R3 seed and F2 seed from canola plants transformed with the fasA and pptl genes as described above in Example 5.

The event that showed a significant increase in oil in the greenhouse (BN_G1216) is included in a randomized complete bloc field test. This event did not show a positive phenotype as an R1 transplant in a first field trial (described above in Example 6). However, because field conditions are variable and have been shown to affect phenotype, and because of the positive phenotype in the greenhouse trial, this second field trial is determined to be warranted. In this second field trial, the gene positive line is compared to its null segregant to determine phenotype. Ebony is included in this experiment as a varietal control. The resulting seed is analyzed for oil and protein and the data is analyzed for statistical differences as described above in Example 6. The results of the R3 seed corroborate the results from the greenhouse grown R2 seed, described above in Example 6.

Three additional events, BN_G1223, BN_G1216, and BN_G1239, that were western blot positive but contained multiple gene copies, are crossed to the variety Ebony in order to produce null segregants from an F1 population of seed that contained gene positive as well as the null segregants. The 3 out-crossed events, BN_G1233xEbony, BN_G1239xEbony, and BN_G1216xEbony, are grown in the field and greenhouse as F1 single plants. They are randomized as positive and negative selections and are grown with Ebony control lines. These events are individually isolated to produce selfed seed. The resulting F2 seed is harvested and analyzed for oil and protein, and the data is analyzed for statistical differences, as described above. The results of the F2 seed corroborate the results from the greenhouse grown R2 seed, described above in Example 6.

### EXAMPLE 8

This example describes the isolation, cloning and sequencing of the Lipomyces starkeyi multifunctional FAS I gene. Total RNA was isolated from the high oil yeast species Lipomyces starkeyi (L.s.) (strain ATCC 56305) using standard methods. First and second strand cDNA was then prepared from the total RNA using a SMART PCR cDNA Synthesis Kit from Clontech and then size fractionated by gel electrophoresis using 0.8% agarose gel in TBE buffer. A slice containing the cDNAs 2.5 kb and larger was cut out of the gel and the cDNAs recovered using a QIAquick DNA Extraction Kit from Qiagen. The cDNAs were ligated into pCR2.1 (Invitrogen) and transformed into TOP10 cells using the manufacturer's protocols. Partial sequencing of the cDNA library revealed the presence of several FAS I clones, the longest of which contained the complete 3′ end of the open reading frame of FAS1 (2780 bp) from residues 3469 to 6249. Additional first strand cDNA template was prepared from total RNA using a GeneRacer™ Kit and GeneRacer™ SuperScript II reverse transcriptase from Invitrogen following the manufacturers protocols. Rapid amplification of cDNA ends (RACE) as outlined in the Invitrogen GeneRacer™ Kit was then used to amplify the 5′ end of the Ls FAS gene from the new cDNA template by utilizing the generic 5′ RACE primer provided with the kit for the 5′ end and the L.s FAS I gene specific primer 20267 (SEQ ID NO:50). The RACE amplified DNA was then cloned into the pCR4Blunt-TOPO vector using the Invitrogen Zero Blunt® TOPO® PCR Cloning Kit and transformed into Top10 cells as outlined in the manufacturers protocols. The resultant clones were screened for the appropriate insert sizes by digestion with restriction endonucleases and further confirmed by DNA sequencing. The full-length L.s. FAS I gene (SEQ ID NO:59) was then obtained as a single contiguous piece of DNA by PCR amplification using Pfx DNA polymerase with the PCR primers 20425 (SEQ ID NO:52) and 20775 (SEQ ID NO:51) which introduce SfiI restriction enzyme sites 5′ and 3′ of the start-ATG and TAG-stop codons respectively, for cloning into expression vectors. The polypeptide sequence of the L.s. FAS I is given in SEQ ID NO:60. Primers used are shown in Table 2.

TABLE 2
Primers utilized for cloning of
Lipomyces starkey FAS I.

| | | Sequence |
|---|---|---|
| Primer # | Name | (SEQ ID NOs: 50-52) |
| 20267 | LsFAS1 537 | 5′ ATGCCTCACCGTTGTTCCCG |
| | 5′race primer | AC 3′ |
| 20775 | LsFAS1 | 5′ GGCCGAGGCGGCCTAAGCAGTCT |
| | | CATACTTCTC 3′ |
| | end with stop | |
| 20425 | LsFAS2gfpATG | 5′ GGCCATTACGGCCATGTACGCTG |
| | | GCGCTGAG 3′ |

### EXAMPLE 9

This example describes the isolation, cloning and sequencing of the Lipomyces starkeyi multifunctional FAS II gene. Total RNA was isolated from the high oil yeast species Lipomyces starkeyi (L.s.) (ATCC 56305) using standard methods. First strand cDNA template was prepared from total RNA using a GeneRacer™ Kit and GeneRacer™ SuperScript II reverse transcriptase from Invitrogen following the manufacturers protocols. FAS II gene sequences from Saccharomyces cerevisiae (Sc) Schizosaccharomyces pombe (Sp) Candida albicans (Ca) and Brevibacterium ammoniagenes (Ba) were aligned and used to design degenerate oligos #20368 (SEQ ID NO:53) and 20369 (SEQ ID NO:54). PCR amplification using the cDNA template and degenerate primers 20368 and 20369 produced a 1.65 kb fragment which was cloned in into pCR4 with TOPO TA pCR4 cloning kit from Invitrogen, and confirmed as a FAS II gene fragment by DNA sequencing. 5′ and 3′ rapid amplification of cDNA ends (RACE) was performed using a GeneRacer™ Kit from Invitrogen to independently amplify the 5′ and 3′ ends of the FAS II gene extending out from the ends of the 1.65 kb Ls FAS II clone. Specifically, the 3′ end of the FAS II gene was isolated by 3′ RACE reactions using the generic 3′ RACE primer from the GeneRacer Kit (Invitrogen) and primer 20595 (SEQ ID NO:55) matching the 3′ end of the 1.65 kb Ls FAS II clone. The 5′ end of the FAS II gene was isolated by 5′ RACE using the generic 5′ RACE primer from the GeneRacer Kit (Invitrogen) and primer 21631 (SEQ ID NO:56) matching the 5′ end of the 1.65 kb Ls FAS II clone. PCR was used to assemble the full-length Ls FAS II gene (SEQ ID NO:61) for cloning into expression vectors using flanking SfiI sites introduced by PCR. Primer 21749 (SEQ ID NO:58) was used to introduce an SfiI site upstream of the ATG, a modified Kozak sequence, and introduce an alanine codon after the ATG for improved expression in corn, and primer 20825 (SEQ ID NO:57) was used to introduce an SfiI site downstream of the stop codon. The polypeptide sequence of L.s. FAS II is given in SEQ ID NO:62. Primers used are shown in table 3.

TABLE 3

Primers utilized for cloning of
Lipomyces starkey FAS II.

| | |
|---|---|
| Primer # | Sequence (SEQ ID NOs: 53-58) |

| | |
|---|---|
| 20368 | 5′GCCRTTNADCATCCANGC 3′ |
| 20369 | 5′ GAYGANAARGAYRTNAARGC 3′ |
| 20595 | 5′ GCGCTACTTCCATTGAGTCTG 3′ |
| 21631 | 5′ ATGTGTCCCCACGCTTCTCC 3′ |
| 20825 | 5′ TGGCCGAGGCGGCTTAAACCAACTCTGCAACAGC 3′ |
| 21749 | 5′ GGCCATTACGGCCAACAATGGCGCGTCCCGAGACTGAGC |
| | A 3′ |

The present invention is not limited to the precise details shown and set forth hereinabove, for it should be understood that many variations and modifications may be made while still remaining within the spirit and scope of the present invention defined by the claims.

What is claimed:

1. An isolated nucleic acid sequence selected from the group consisting of:
(a) a nucleic acid sequence with at least about 85% identity to SEQ ID NO:59 or SEQ ID NO:61;
(b) a nucleic acid sequence encoding a polypeptide sequence with at least about 85% identity to SEQ ID NO:60 or SEQ ID NO:62;
(c) a nucleic acid sequence that hybridizes to SEQ ID NO:59 or SEQ ID NO:61 under conditions of 1×SSC, and 65° C. and encodes a polypeptide that displays multifunctional fatty acid synthase activity; and
(d) the complement of (a)-(c).

2. A transgenic plant comprising the nucleic acid sequence of claim 1.

3. The plant of claim 2, further comprising a second heterologous nucleic acid molecule encoding a phosphopantetheine:protein transferase enzyme.

4. The plant of claim 2, wherein the nucleic acid sequence encodes a multifunctional fatty acid synthase comprising the amino acid sequence of SEQ ID NO: 60 or SEQ ID NO:62.

5. The plant of claim 2, wherein the nucleic acid molecule encodes a multifunctional fatty acid synthase comprising the amino acid sequence of SEQ ID NO:60.

6. The plant of claim 3, wherein the nucleic acid molecule encoding a phosphopantetheine:protein transferase enzyme comprises SEQ ID NOs: 4, 33, 35, 37, 39, 41, and 43.

7. The plant of claim 2, wherein the polypeptide encoded by the nucleic acid sequence increases oil levels in the seed of a plant that has been transformed with said nucleic acid.

8. The plant of claim 3, that further comprises a promoter that is operably linked to the nucleic acid encoding a multifunctional fatty acid synthase or the second nucleic acid encoding a phosphopantetheine:protein transferase enzyme, wherein the promoter is functional in a plant cell.

9. The plant of claim 8, wherein the promoter provides expression substantially within plant seeds of the multifunctional fatty acid synthase or the phosphopantetheine:protein transferase enzyme.

10. The plant of claim 2, wherein the multifunctional fatty acid synthase is substantially located in the cytosol of a plant cell.

11. The plant of claim 2, selected from the group consisting of: Brassica sp., canola, mustard, crambe, oilseed rape, rapeseed, Arabidopsis thaliana, soybean, safflower, sunflower, corn, rice, barley, millet, rye, wheat, oat, alfalfa, sorghum, soybean, grape, cotton, flax (linseed), castor bean, sesame, oil palm, jojoba, peanut, and Chinese tallow tree.

12. A method for producing a transgenic plant with increased oil content comprising: expressing the nucleic acid sequence of claim 1 in the plant.

13. The method of claim 12, further comprising introducing a second nucleic acid molecule encoding a phosphopantetheine:protein transferase enzyme into the plant cell.

14. The method of claim 12, wherein the nucleic acid sequence comprises SEQ ID NO:59 or SEQ ID NO:61.

15. The method of claim 12, wherein the nucleic acid sequence encodes a multifunctional fatty acid synthase polypeptide comprising an amino acid sequence with at least about 85% sequence identity to a sequence selected from the group consisting of SEQ ID NOs:60 and 62.

16. The method of claim 15, wherein the polypeptide comprises SEQ ID NO: 60 or SEQ ID NO:62.

17. The method of claim 13, wherein the second nucleic acid molecule encoding a phosphopantetheine:protein transferase enzyme encodes an amino acid comprising a protein selected from the group consisting of SEQ ID NOs: 4, 33, 35, 37, 39, 41, and 43.

18. The method of claim 12, wherein the polypeptide encoded by the nucleic acid sequence produces increased oil levels in a seed of the plant.

19. The method of claim 13, wherein the nucleic acid sequence of claim 1 or the second nucleic acid molecule encoding a phosphopantetheine:protein transferase enzyme further comprises a promoter that is operably linked thereto, wherein the promoter is functional in the plant cell.

20. The method of claim 19, wherein the promoter is a globulin promoter, a zein promoter, an oleosin promoter, an ubiquitin promoter, a CaMV 35S promoter, a CaMV 19S promoter, a nos promoter, an Adh promoter, a sucrose synthase promoter, a tubulin promoter, a napin promoter, an actin promoter, a cab promoter, a PEPCase promoter, a 7S-alpha′-conglycinin promoter, an R gene complex promoter, a tomato E8 promoter, a patatin promoter, a mannopine synthase promoter, a soybean seed protein glycinin promoter, a soybean vegetative storage protein promoter, or a root-cell promoter.

21. The method of claim 19, wherein the promoter provides expression substantially within plant seeds of the multifunctional fatty acid synthase or the phosphopantetheine:protein transferase enzyme.

22. The method of claim 12, wherein the multifunctional fatty acid synthase is substantially located in the cytosol of the plant cell.

23. A method of producing a plant oil, comprising the steps of:
a) growing an oilseed plant, the genome of which contains a nucleic acid sequence according to claim 1, to produce oil-containing seeds; and
b) extracting oil from the seeds.

24. The method of claim 23, wherein the nucleic acid sequence encodes a polypeptide comprising SEQ ID NO:60 or SEQ ID NO:62.

25. A method of producing a plant oil, comprising the steps of:
a) growing the oilseed plant of claim 23, the genome of which further contains a nucleic acid sequence encoding a phosphopantetheine:protein transferase, to produce oil-containing seeds; and
b) extracting oil from the seeds.
